(19) United States
(12) Patent Application Publication
(10) Pub. No.: US 2017/0217914 A1
(43) Pub. Date: Aug. 3, 2017

(54) Novel Triarylethylene Compounds and Methods Using Same

(71) Applicants: The Penn State Research Foundation, University Park, PA (US); Apeejay Stya University, Haryana (IN)

(72) Inventors: Arun K. Sharma, Hummelstown, PA (US); Manoj Pandey, Hummelstown, PA (US); Shantu Amin, Union City, NJ (US); Mohinder P. Mahajan, Gurgaon (IN); Gurleen Kaur, Amritsar (IN)

(21) Appl. No.: 15/500,800

(22) PCT Filed: Jul. 28, 2015
(86) PCT No.: PCT/US15/42357

§ 371 Date: Jan. 31, 2017

Related U.S. Application Data
(60) Provisional application No. 62/033,496, filed on Aug. 5, 2014.

(51) Int. Cl. C07D 295/215; A61K 31/085; C07C 43/23; A61K 31/255; A61K 31/655; A61K 31/27; C07C 43/225; A61K 31/216; A61K 31/155; A61K 31/5375; A61K 31/4453; A61K 31/40; A61K 31/165; C07C 309/66; C07C 247/04; C07C 247/10; A61K 31/137; C07C 217/62; C07C 271/44; C07C 39/373; C07C 233/56; C07C 275/24; C07C 275/26; A61K 45/06

(52) U.S. Cl. CPC C07D 295/215; A61K 45/06; A61K 31/085; C07C 43/23; A61K 31/255; A61K 31/655; A61K 31/27; C07C 43/225; A61K 31/216; A61K 31/155; A61K 31/5375; A61K 31/4453; A61K 31/40; A61K 31/165; C07C 309/66; C07C 247/04; C07C 247/10; A61K 31/137; C07C 217/62; C07C 271/44; C07C 39/373; C07C 233/56; C07C 275/24; C07C 275/26

(57) ABSTRACT

The present invention includes compounds useful in preventing or treating cancer in a subject in need thereof. The present invention also includes methods of preventing or treating cancer in a subject in need thereof by administering to the subject a therapeutically effective amount of a compound of the invention.

### CROSS-REFERENCE TO RELATED APPLICATIONS

The present application claims priority to U.S. Provisional Application No. 62/033,496, filed Aug. 5, 2014, which is hereby incorporated by reference in its entirety herein.

### BACKGROUND OF THE INVENTION

Cancer is known to be one of the leading causes of death worldwide, and it accounts for approximately 8.2 million deaths (DeSantis et al., 2014, CA Cancer J. Clin. 64:52-62). According to a recent survey, it is expected that the new cancer cases will increase fivefold by 2025 (DeSantis et al., 2014, CA Cancer J. Clin. 64:52-62). It is not just one disease but a diverse group of diseases, an uncontrollable growth of abnormal cells that invade one tissue or organ and have the ability to spread in other tissues at much higher rate, process known as metastasis. In this multistep process, one of the reasons for the uncontrolled cell proliferation is the damage to genes i.e. mutations. Cells that divide are at a higher risk of acquiring mutations than cells that do not divide. Cancer is generally rare in tissues in which cells do not divide, like nerve tissue. In contrast, the cancer is more common in tissues in which cells divide frequently, such as with breast, skin, colon, and uterine tissues.

Breast cancer is more common in the age group of 14-30 (younger cells more prone to carcinogens, early menarche, late or less breast feeding etc.) and in the age group of 40 and above (lifestyle, late menopause etc.). In 2013, an estimated 232,340 new cases of invasive and an estimated 64,640 additional cases of noninvasive breast cancer were diagnosed among women globally and approximately, 39,620 women were estimated to die because of this deadly disease (DeSantis et al., 2014, CA Cancer J. Clin. 64:52-62). Breast cancer is the most common invasive cancer in females worldwide. It accounts for 16% of all female cancers and 22.9% of invasive cancers in women. It represents one third of all the cancers diagnosed in pre-menopausal and post-menopausal women and is the second leading cause of cancer death amongst women (Cancer Facts and Figures, 2015, American Cancer Society, Atlanta). In addition, it is a leading cause of premature death in women. Estrogen plays a crucial role in promoting the growth of hormone dependent breast cancer in pre and post-menopausal women breast cancers (Tomao et al., 2015, OncoTargets and therapy 8:177-193). Breast cancers that have estrogen or progesterone receptors are referred to as ER-positive (ER+) or PR-positive (PR+), respectively. If either type of receptor is absent, the cancer is said to be hormone receptor negative (Vici et al., 2015, Cancer treatment reviews 41:69-76). Hormone therapy is recommended for the patients suffering from breast cancer due to the involvement of hormone receptors (ER and PR) (Hart et al., 2015, Nature Reviews Clinical Oncology; Chlebowski and Anderson, 2015, Therapeutic advances in drug safety 6:45-56).

The main strategy for the treatment of breast cancer involves the mechanism of blocking the growth/amount of estrogen produced to kill the cancer cells. Breast tissue is particularly sensitive to developing cancer for several reasons. The female hormone estrogen stimulates breast cell division leading to the increase in risk of permanent damage to DNA. The physiological effects of estrogen are regulated by two estrogen receptor (ER) subtypes, ERα and ERβ (Huang et al., 2014, Mol. Cell. Endocrinol.). ERα is normally expressed in the breast cancer cells, and is an important target for the development of new anti-breast cancer agents (Huang et al., 2015, Molecular and cellular endocrinology). Although, its second isoform, ERβ, is expressed in brain, kidney, bone and lungs, and possess only 55% amino acid similarity with the ERα, all but two amino acids of their binding pockets are the same ((Pike et al., 1999, EMBO J. 18:4608-4618; Nettles et al., 2007, EMBO reports 8:563-568). The role of ERβ as anti-proliferative and pro-apoptotic receptor has also been highlighted (Leygue and Murphy, 2013, Endocr. Relat. Cancer 20:127-139; Hapangama et al., 2015, Human reproduction update 21:174-193; Pike, Clinical endocrinology & metabolism 20:1-14). This category mainly involves the use of aromatase inhibitors (such as Anastrozole and Letrozole, FIG. 1) and SERMs (like Tamoxifen, Toremifene, Raloxifene and Ospemifene, FIG. 2) that work almost on the same principle (Martinkovich et al., 2014, Clinical interventions in aging 9:1437-1452; Olin and St. Pierre; Annals Pharmacotherapy 48:1605-1610). The main advantage of these two modes of action over other modes is the use of non-steroidal drugs instead of steroidal which may interfere with the normal biological balance. Aromatase inhibitors are successful in post-menopausal cancers in blocking the release of enzyme aromatase and ultimately estrogen, however, its use may stop the release of aromatase enzyme necessary in bones and can lead to Osteoporosis (Becorpi et al., 2014, J. Ital. Soc. Osteoporosis, Mineral Metabol. Skeletal Diseases 11:110-113). Also, the resistance to these agents has become a major clinical obstacle.

Selective Estrogen Receptor Modulators (SERMs), selective non-steroidal estrogen agonists/antagonists, have been known for over two decades and have shown huge clinical applications in the treatment of both pre- and post-menopausal breast cancer, osteoporosis and cholesterol related problems. Some of the known SERMs like Tamoxifen, Toremifene and Raloxifene, showed some major concerns involving uterine cancer, endometrial cancer, hot flashes, vaginal dryness etc. (Ellmen et al., 2003, Breast Cancer Res. Treat. 82:103-111; Taras et al., 2001, J. Steroid Biochem. Mol. Biol. 77:271-279). Latest in the series is the US-FDA approved Ospemifene, with the brand name Osphena, for the treatment of Dyspareunia (VVA—vulvar and vaginal atrophy), showing promising pharmacological profile compared to its known derivatives (Wurz et al., 2014, Clin. Interv. Aging. 9:1939-1950). It has shown all the possibilities of being the ideal SERM, the one which can act as an agonist in heart, bone and vagina, and antagonist in breast and uterus.

It has been observed that the triarylethylene framework forms the backbone and is the one responsible for mimicking the effect of natural estrogen that can bind to the estrogen receptor to produce its effects (agonist/antagonist) (Ray, 2004, Drugs of the Ruture 29:185-203). It was inferred that this framework acts as an estrogen agonist and the two alkyl chains attached to this framework are responsible for its behaviour as an antagonist (full/partial). The effect of alkyl chains lengthening has been demonstrated in case of Tamoxifen and Raloxifene, acting as partial antiestrogens, and the drugs such as ICI 182,780 and RU 58668 acting as full antiestrogens (Schneider et al., 1986, J. Cancer Res. Clin. Oncol. 112:258-265). It has also been reported that almost all known aromatase inhibitors and SERMs selectively lower the risk of ER-positive breast cancers without affecting the ER-negative breast cancers (Files et al., 2010, Mayo Clin. Proc. 85:560-566). Compounds with triarylethylene pharmacophore have been reported with promising biological activities in estrogen-dependent disorders such as breast cancer, osteoporosis, CNS/CVS and in fertility regulation (Suprabhat, 2004, Drug Future 29:185-203). SERM medications act by blocking estrogen from attaching to the estrogen receptor on the cancer cells, slowing the growth of tumors and killing tumor cells. An ideal SERM, useful as anti-breast cancer agent, is the one having antiestrogenic effect in breast and uterus, while estrogenic effect in bone and heart. SERMs, which can be used as anti-breast cancer agents for the treatment in both pre- and postmenopausal women, include tamoxifen, raloxifene (Evista) and toremifene (Fareston) (Descoteaux et al., 2008, Steroids 73:1077-1085).

One of the drugs recently approved by the US FDA, Ospemifene (generic name), is used for the treatment of moderate to severe dyspareunia, a symptom of vaginal and vulvar atrophy, due to menopause. It has been proven as a novel SERM that acts as an agonist by mimicking estrogen in brain, bone and vagina and acts as an antagonist in uterus and breasts (Adsule et al., 2010, Bioorg. Med. Chem. Lett. 20:1247-1251). Its biological actions are mediated through binding to estrogen receptors resulting in the activation of estrogenic pathways in some tissues (agonism) and blockade of estrogenic pathways in others (antagonism). The efficacy and safety of this drug was demonstrated in three clinical trials. Recent reports have also shown its bioactivity in the prevention of osteoporosis and as an anti-breast cancer agent (Kumamoto et al., 2010, Helv. Chim. Acta 93:2109-2114; Havrylyuk et al., 2011, Arch. Pharm. 344:514-522). It has exhibited a promising pharmacological profile having estrogenic effects on bone and the cardiovascular system, minimal effects on the uterus and antiestrogenic effects on breasts, unlike its other active metabolites and derivatives: for example, Tamoxifen has an adverse effect on endometrium causing endometrial cancer (Ellmen et al., 2003, Breast Cancer Res. Treat. 82:103-111), and Raloxifene causes hot flashes, insomnia, dizziness, melancholy etc. (Taras et al., 2001, J. Steroid Biochem. Mol. Biol. 77:271-279). Also, it has been reported that the presence of the chlorine group in Ospemifene reduces the antiestrogenic activity while the introduction of azide group in a number of organic molecules enhances the anticancer activity (Stygar et al., 2003, Reprod. Biol. Endocrinol. 1:40). Thus, the design and the synthesis of novel SERMs as effective anti-breast cancer agents is considered to be of great value.

There is a need in the art to identify novel compounds which are useful for the treatment of cancer, in addition to other diseases and disorder, and do not cause deleterious side effects in the subject. The present invention fulfills this need.

### SUMMARY OF THE INVENTION

The present invention relates to a compound of formula (I):

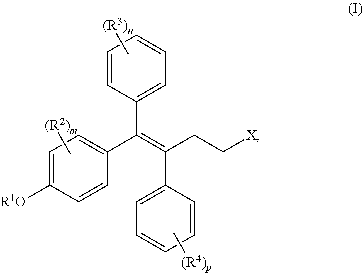

wherein in formula (I):

R¹ is selected from the group consisting of H and alkyl, wherein the alkyl group is optionally substituted;

each occurrence of R², R³, and R⁴ is independently selected from the group consisting of H, —C₁-C₆ alkyl, —C₁-C₆ fluoroalkyl, —C₁-C₆ heteroalkyl, F, Cl, Br, I, —CN, —NO₂, —OR⁴, —SR⁴, —S(═O)R⁴, —S(═O)₂R⁴, —NHS(═O)₂R⁴, —C(NH)(NH₂), —C(═O)R⁴, —OC(═O)R⁴, —CO₂R⁴, —OCO₂R⁴, —CH(R⁴)₂, —N(R⁴)₂, —C(═O)N(R⁴)₂, —OC(═O)N(R⁴)₂, —NHC(═O)NH(R⁴), —NHC(═O)R⁴, —NHC(═O)OR⁴, —C(OH)(R⁴)₂, and —C(NH₂)(R⁴)₂;

X is selected from the group consisting of N₃, N(R⁵)(R⁶), Cl, Br, I, and F;

R⁵ and R⁶ are each independently selected from the group consisting of H, —C₁-C₆ alkyl, —C(O)R⁷, and —C(S)R⁷;

R⁷ is selected from the group consisting of OR⁸, N(R⁸)(R⁹), C(O)R⁸, and C(O)N(R⁸)(R⁹);

R⁸ and R⁹ are each independently selected from the group consisting of hydrogen, —C₁-C₆ alkyl, aryl, cycloalkyl, and —C₁-C₆ alkyl-aryl, wherein the alkyl, aryl, cycloalkyl, or alkylaryl group may be optionally substituted, and wherein R⁸ and R⁹ may combine to form a ring, wherein the ring may optionally contain two or more heteroatoms;

m is an integer from 0 to 4;

n is an integer from 0 to 5; and

p is an integer from 0 to 5,

a salt or solvate, and any combinations thereof,

with the proviso that the compound of formula (I) is not

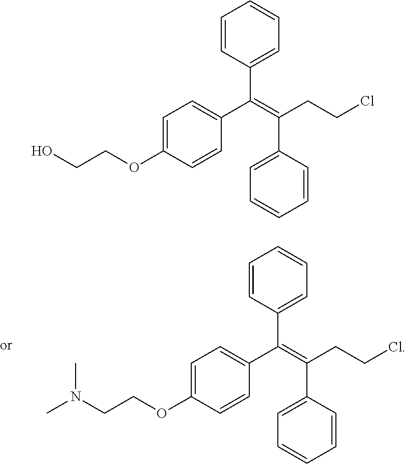

In one embodiment, R¹ is selected from the group consisting of H, methyl, —(CH₂)₂OH, —(CH₂)₂OS(O)₂CH₃, —(CH₂)₂N₃, —(CH₂)₂NH₂, and —(CH₂)₂N(CH₃)₂. In another embodiment, X is N(R⁵)(R⁶). In another embodiment, either R⁵ and R⁶ are each H or R⁵ is H and R⁶ is —C(O)R⁷. In another embodiment, m is 0, n is 0, and p is 0. In another embodiment, the compound is selected from the group consisting of:

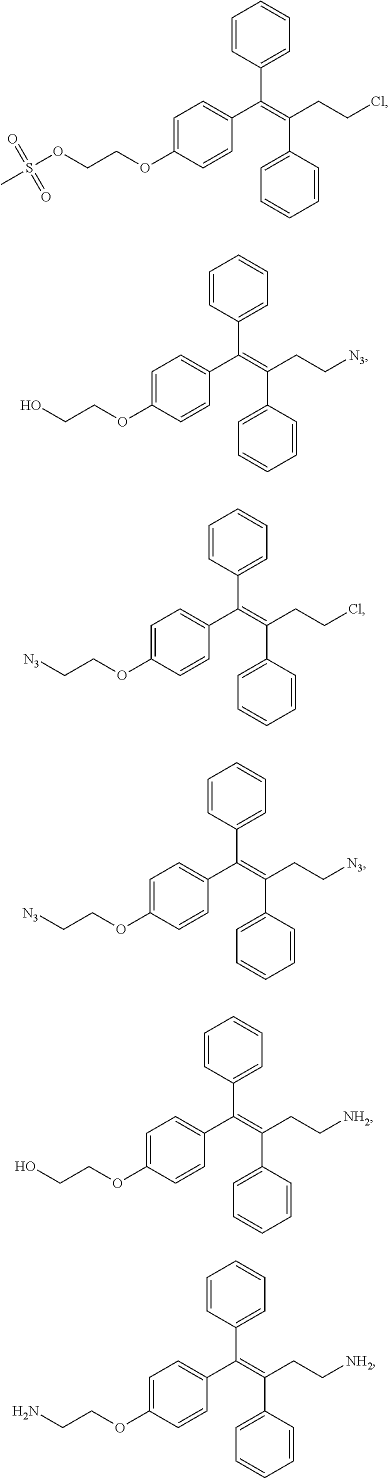

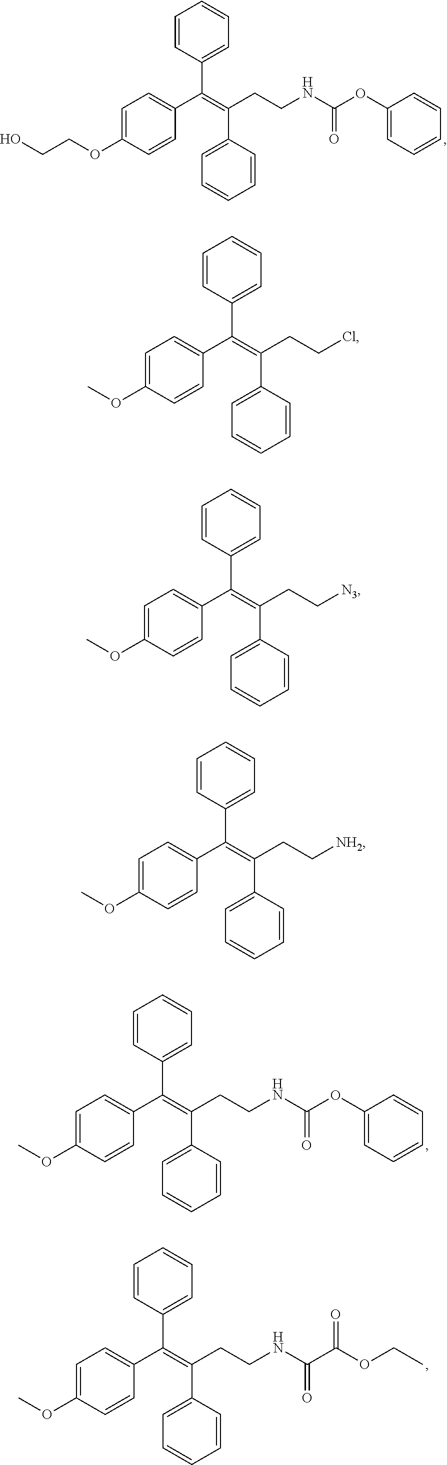

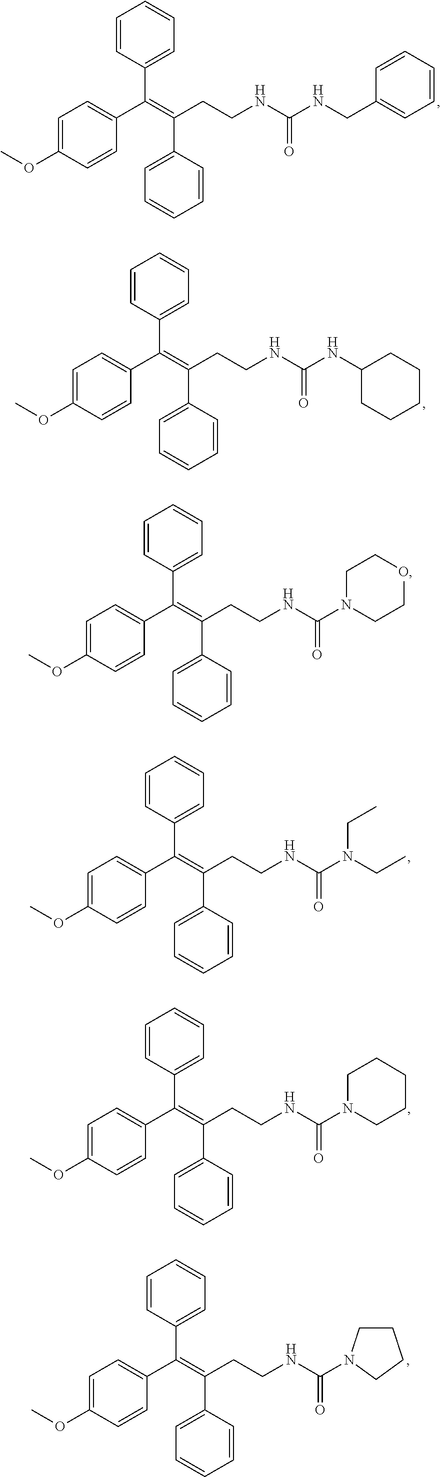

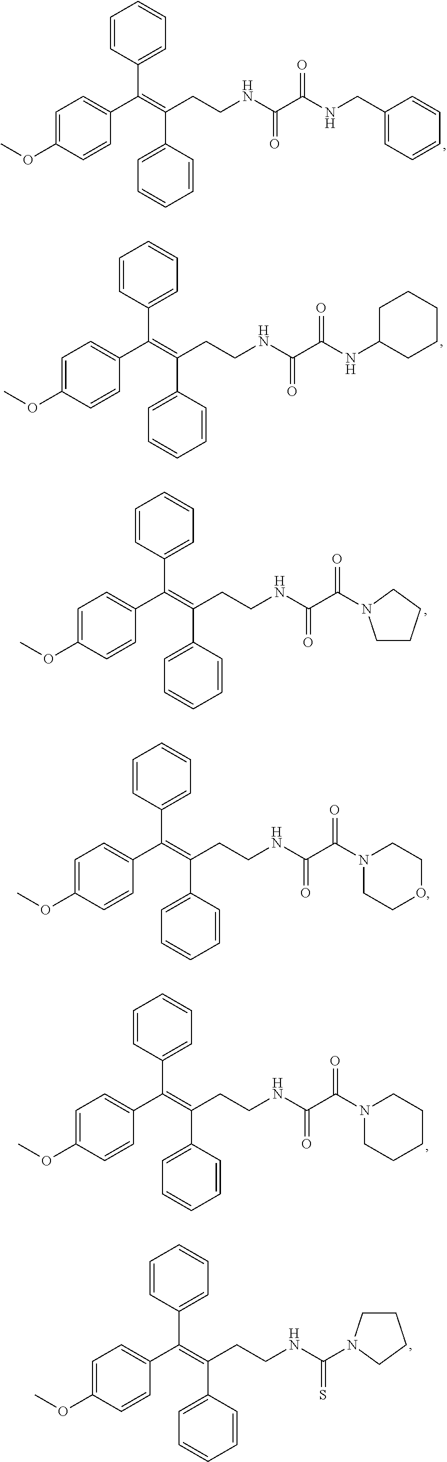

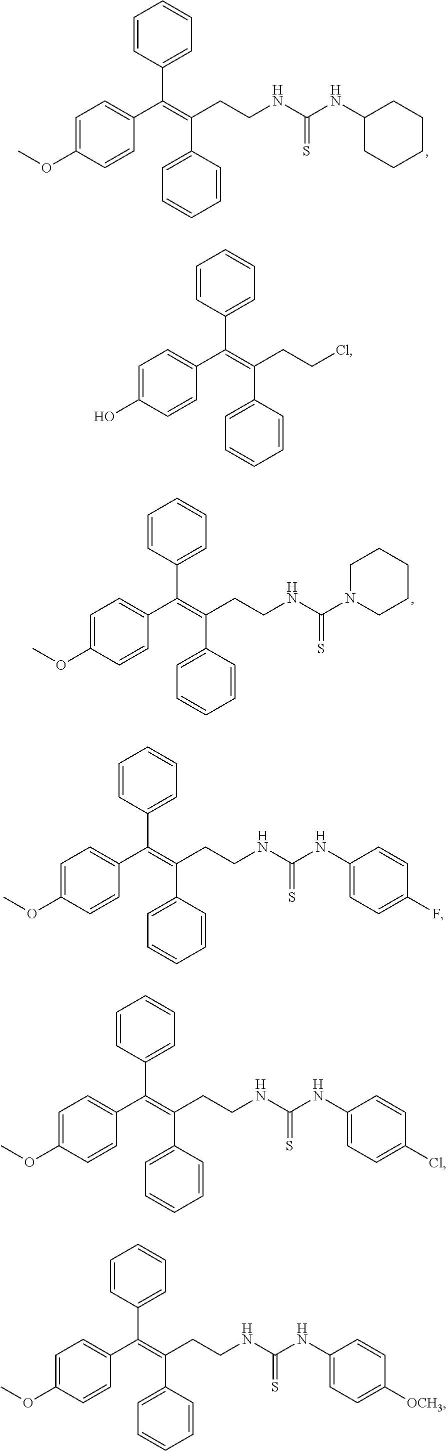

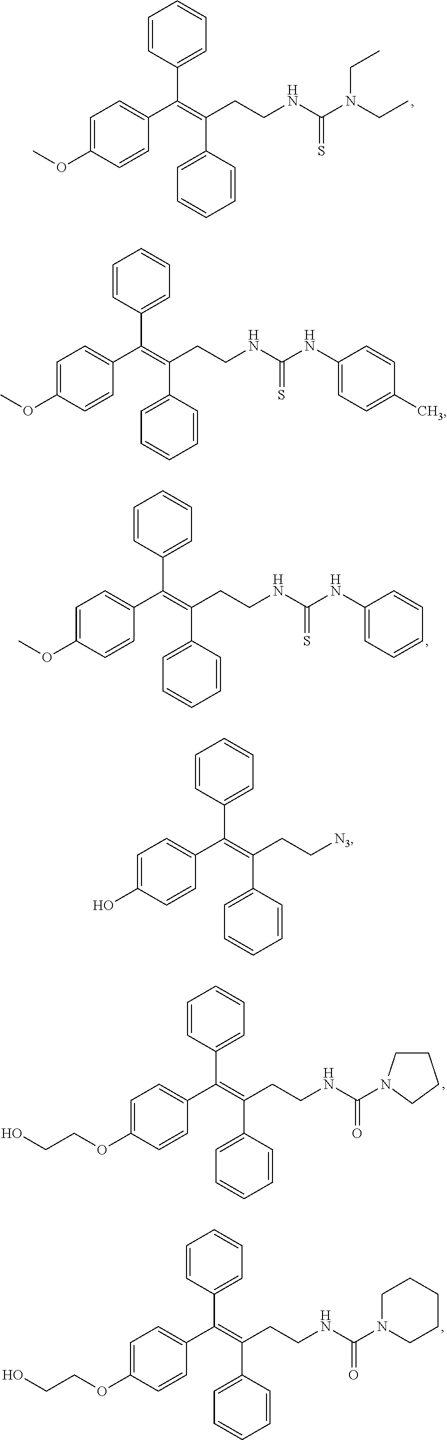

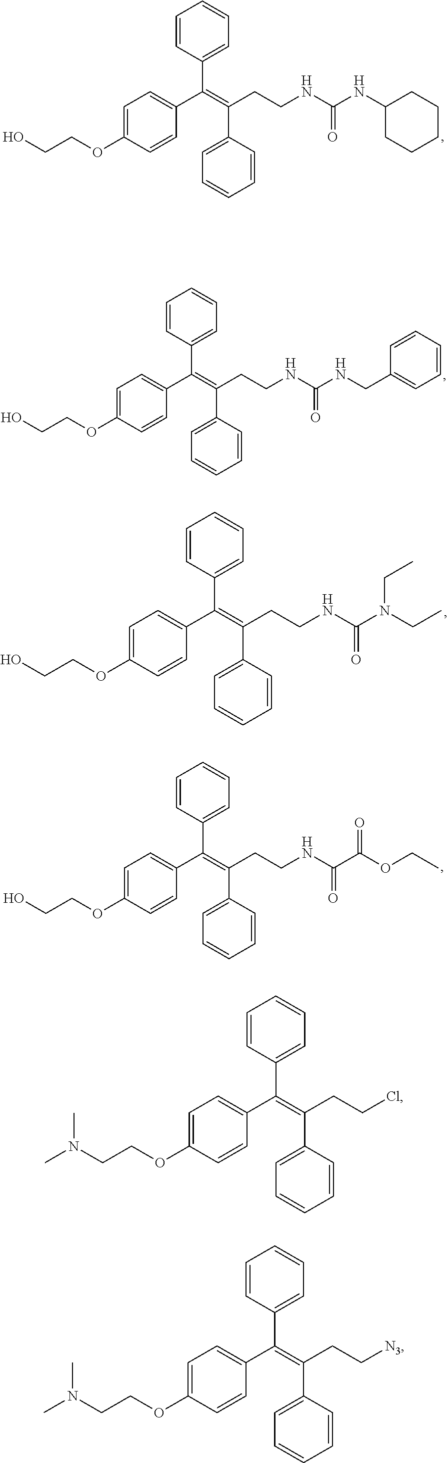

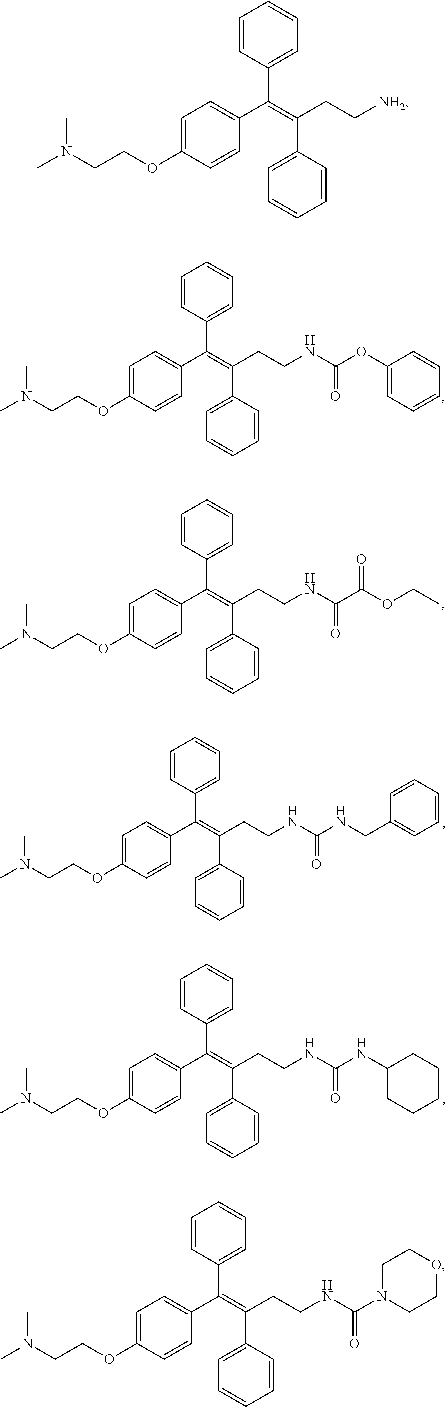

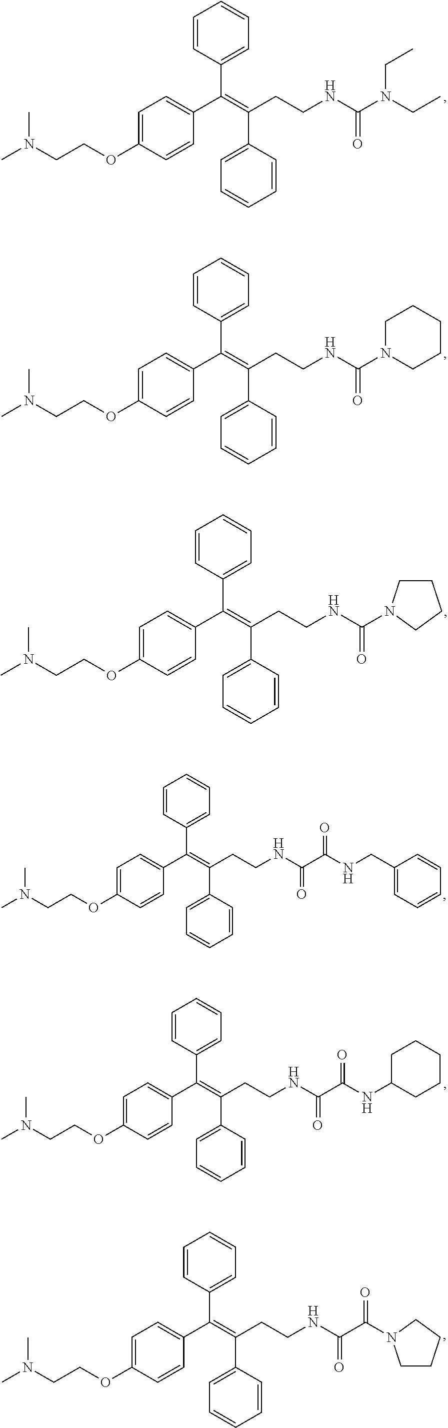

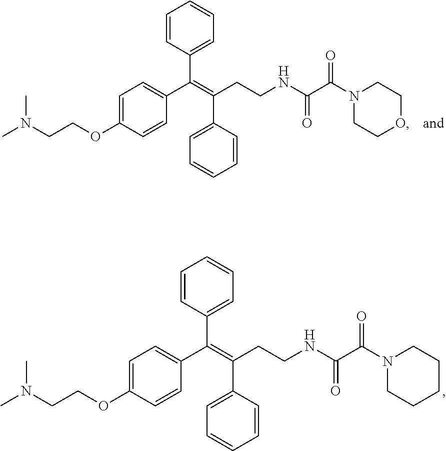 [SMILES: CN(C)CCOc1ccc(/C(=C(/CCNC(=O)C(=O)N2CCOCC2)c2ccccc2)c2ccccc2)cc1 ; CN(C)CCOc1ccc(/C(=C(/CCNC(=O)C(=O)N2CCCCC2)c2ccccc2)c2ccccc2)cc1]

a salt or solvate thereof, and any combinations thereof.

The present invention also relates to a composition comprising a compound of the invention. In one embodiment, the composition further comprises a pharmaceutically acceptable carrier. In another embodiment, the composition further comprises an additional therapeutic agent.

The present invention also relates to a method of preventing or treating cancer in a subject in need thereof. The method includes the step of administering to the subject a therapeutically effective amount of a composition comprising at least one compound of formula (I):

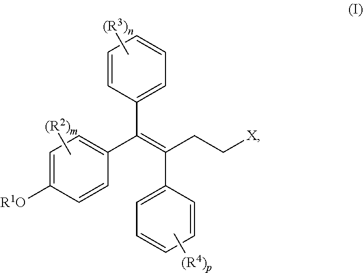

wherein in formula (I):

R¹ is selected from the group consisting of H and alkyl, wherein the alkyl group is optionally substituted;

each occurrence of R², R³, and R⁴ is independently selected from the group consisting of H, —C₁-C₆ alkyl, —C₁-C₆ fluoroalkyl, —C₁-C₆ heteroalkyl, F, Cl, Br, I, —CN, —NO₂, —OR⁴, —SR⁴, —S(═O)R⁴, —S(═O)₂R⁴, —NHS(═O)₂R⁴, —C(NH)(NH₂), —C(═O)R⁴, —OC(═O)R⁴, —CO₂R⁴, —OCO₂R⁴, —CH(R⁴)₂, —N(R⁴)₂, —C(═O)N(R⁴)₂, —OC(═O)N(R⁴)₂, —NHC(═O)NH(R⁴), —NHC(═O)R⁴, —NHC(═O)OR⁴, —C(OH)(R⁴)₂, and —C(NH₂)(R⁴)₂;

X is selected from the group consisting of N₃, N(R⁵)(R⁶), Cl, Br, I, and F;

R⁵ and R⁶ are each independently selected from the group consisting of H, —C₁-C₆ alkyl, —C(O)R⁷, and —C(S)R⁷;

R⁷ is selected from the group consisting of OR⁸, N(R⁸)(R⁹), C(O)R⁸, and C(O)N(R⁸)(R⁹);

R⁸ and R⁹ are each independently selected from the group consisting of hydrogen, —C₁-C₆ alkyl, aryl, cycloalkyl, and —C₁-C₆ alkyl-aryl, wherein the alkyl, aryl, cycloalkyl, or alkylaryl group may be optionally substituted, and wherein R⁸ and R⁹ may combine to form a ring, wherein the ring may optionally contain two or more heteroatoms;

m is an integer from 0 to 4;

n is an integer from 0 to 5; and

p is an integer from 0 to 5,

a salt or solvate thereof, and any combinations thereof.

In one embodiment, R¹ is selected from the group consisting of H, methyl, —(CH₂)₂OH, —(CH₂)₂OS(O)₂CH₃, —(CH₂)₂N₃, —(CH₂)₂NH₂, and —(CH₂)₂N(CH₃)₂. In another embodiment, X is N(R⁵)(R⁶). In another embodiment, either R⁵ and R⁶ are each H or R⁵ is H and R⁶ is —C(O)R⁷. In another embodiment, m is 0, n is 0, and p is 0. In another embodiment, the compound is selected from the group consisting of:

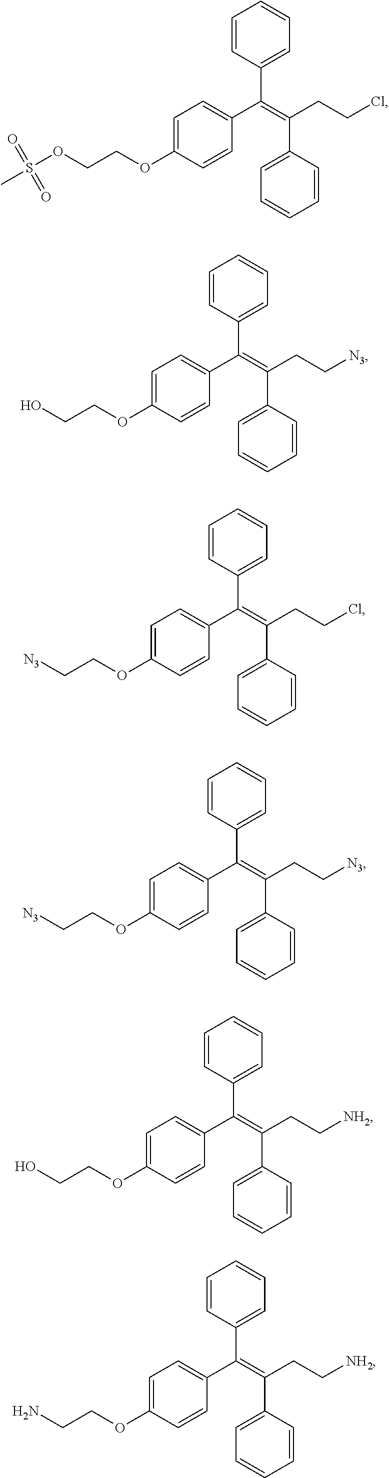

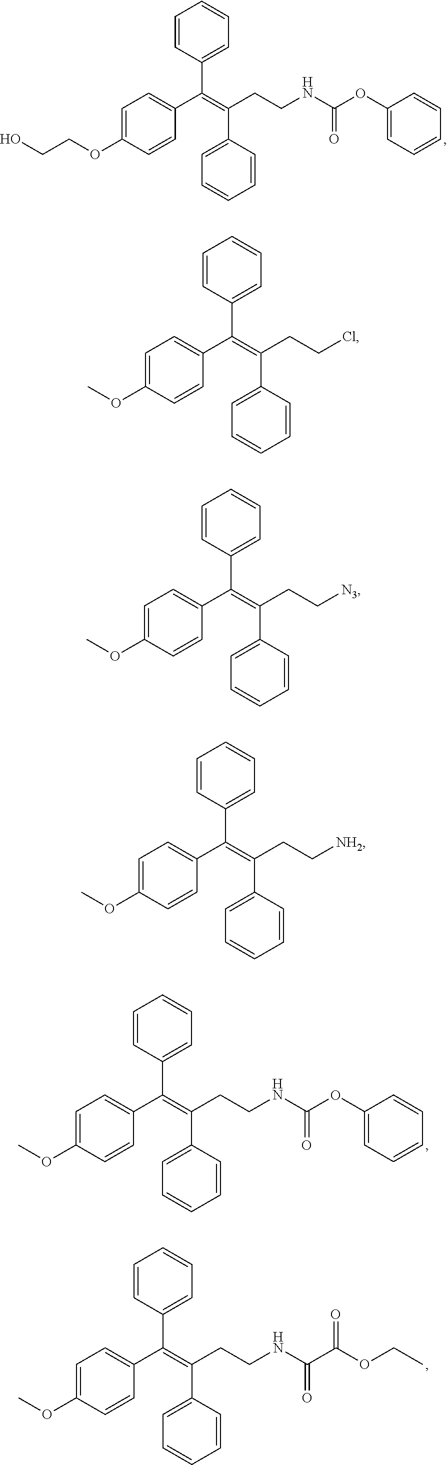

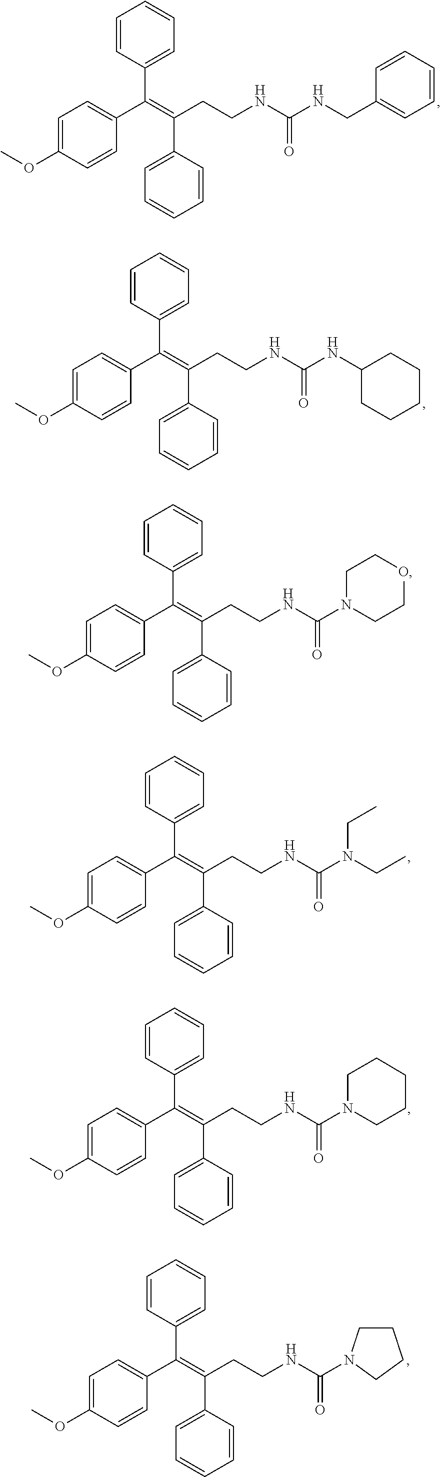

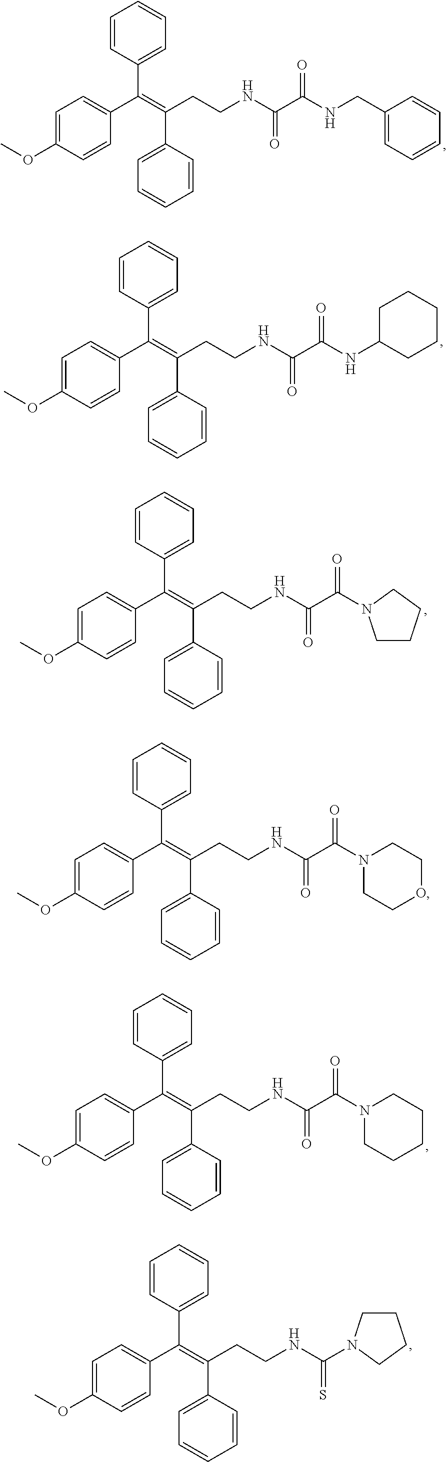

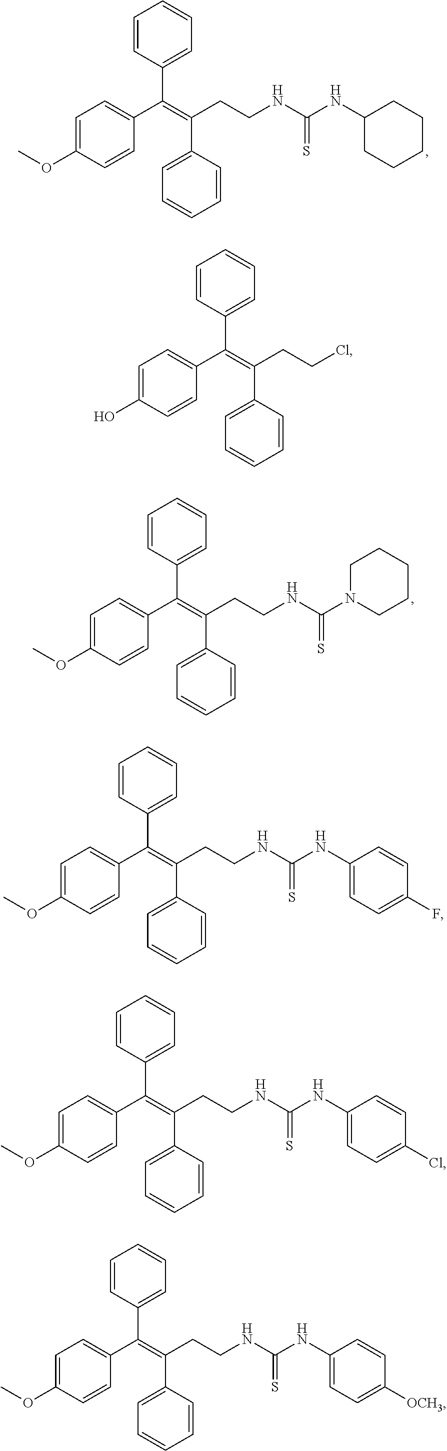

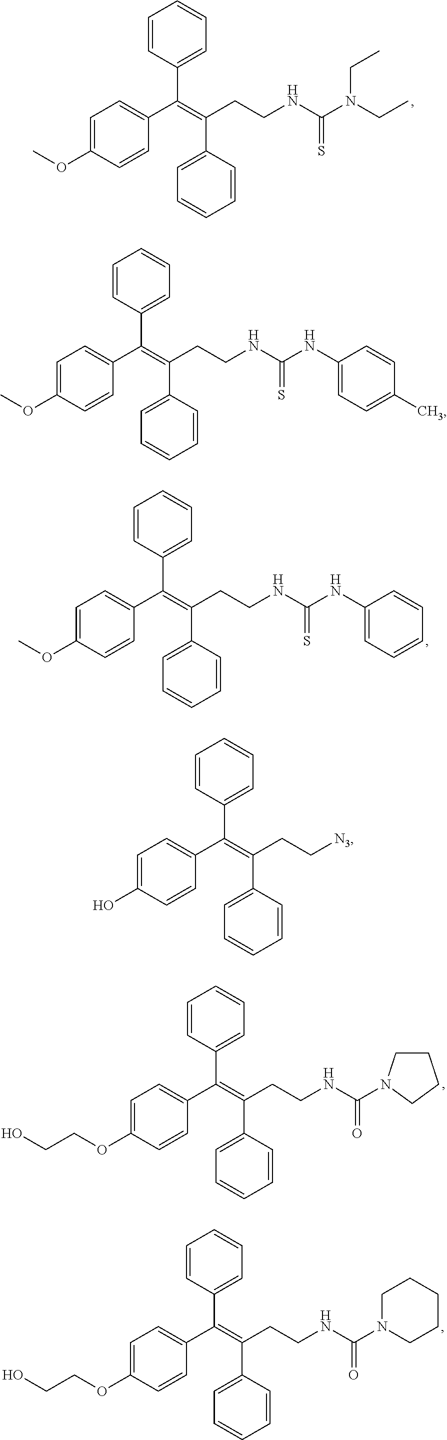

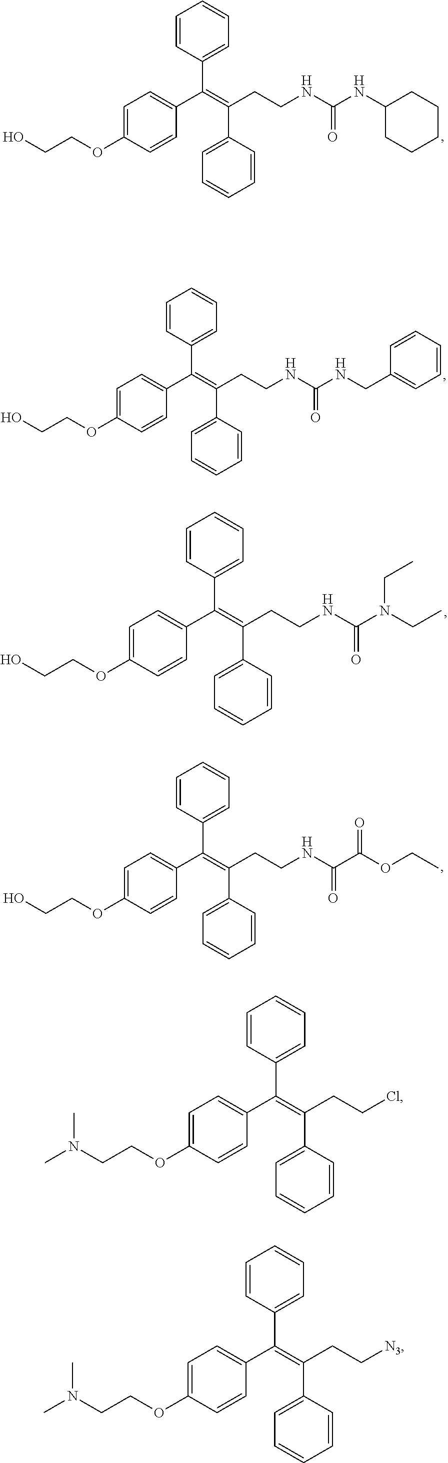

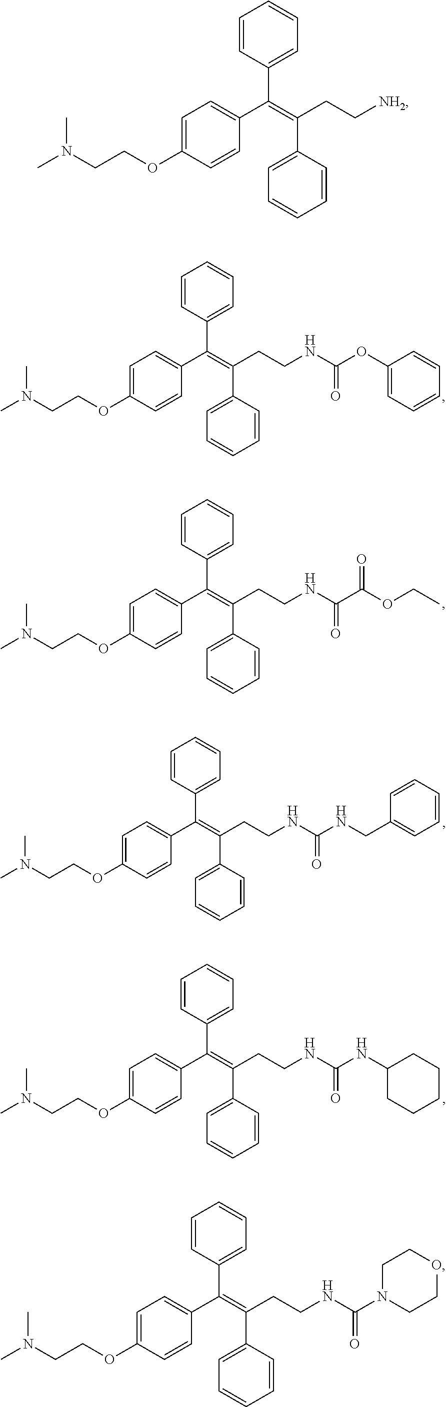

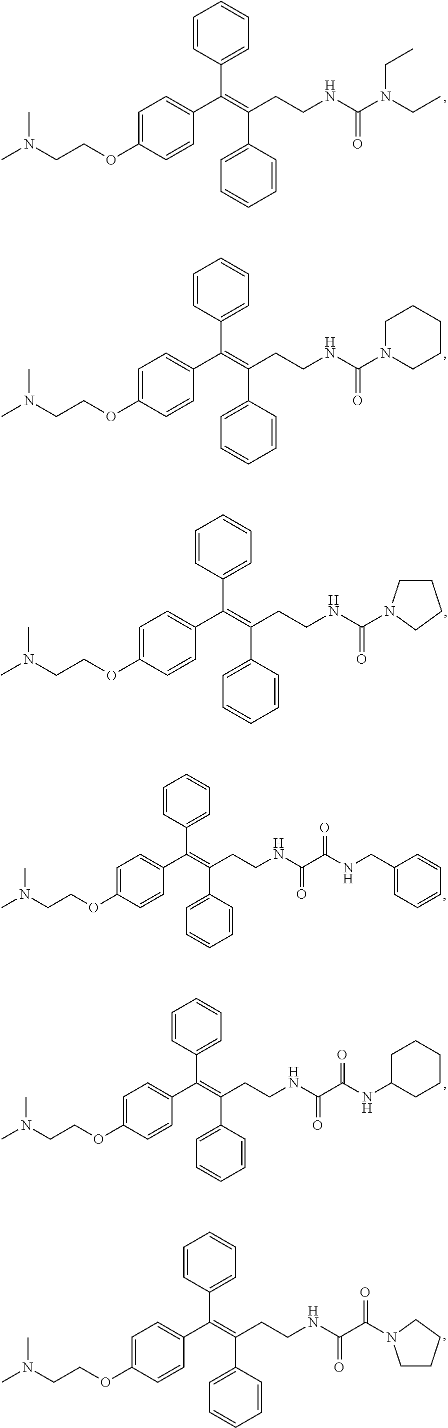

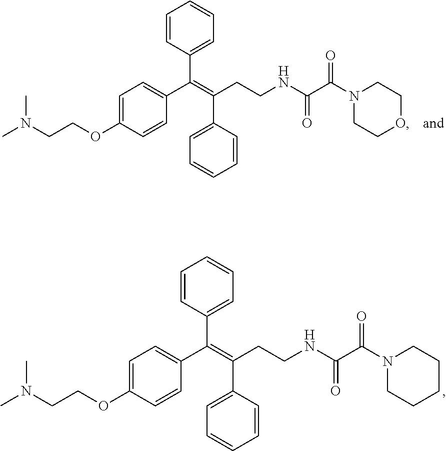 [SMILES: CN(C)CCOc1ccc(/C(=C(/CCNC(=O)C(=O)N2CCOCC2)c2ccccc2)c2ccccc2)cc1 ; CN(C)CCOc1ccc(/C(=C(/CCNC(=O)C(=O)N2CCCCC2)c2ccccc2)c2ccccc2)cc1]

a salt or solvate thereof, and any combinations thereof. In another embodiment, the cancer is selected from the group consisting of lung cancer, colon cancer, melanoma, breast cancer, ovarian cancer, prostate cancer, liver cancer, pancreatic cancer, a CNS tumor, neuroblastoma, leukemia, bone cancer, intestinal cancer, lymphoma, and combinations thereof. In another embodiment, the method further includes the step of administering to the subject at least one additional therapeutic agent. In another embodiment, the therapeutic agent is a chemotherapeutic agent. In another embodiment, the composition and the additional therapeutic agent are co-administered. In another embodiment, the composition and the additional therapeutic agent are co-formulated.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts the structures of two aromatase inhibitors and four important anti-cancer triarylethylene derivatives.

FIG. 2 is a scheme of an exemplary synthesis of compounds 2-8 of the present invention.

FIG. 3 is a graph depicting experimental data demonstrating the effects of Ospemifene and compounds 2-8 of the present invention on the survival of breast cancer cells. Breast cancer cells were treated with increasing concentrations of Ospemifene and compounds 2-8 (0.5-25 μM) for 72 h. Cell viability was then analyzed by the MTT assay. Percentage cell viability was normalized against vehicle treated cells and graph was plotted with maximum concentration (25 μM). Bar diagram is derived from triplicate values of two independent experiments and presented as Mean±SD.

FIG. 4, comprising FIGS. 4A-4E, depicts experimental data demonstrating the effects of Ospemifene, Tamoxifen and compounds 6 and 7 on breast cancer cells survival. Breast cancer cells (MDA-MB-231 and MCF-7) or normal mouse embryonic fibroblast (MEF) cells were treated with increasing concentrations of selected Ospemifene analogs along with Ospemifene and Tamoxifen (0.5e100 mM) for 48 or 96 h. Cell viability was then analyzed by the MTT assay. FIG. 4A is a graph depicting experimental data of the effects of the compounds on MDA-MB-231 cells treated for 48 h. FIG. 4B is a graph depicting experimental data of the effects of the compounds on MDA-MB-231 cells treated for 96 h. FIG. 4C is a graph depicting experimental data of the effects of the compounds on MCF-7 cells treated for 48 h. FIG. 4D is a graph depicting experimental data of the effects of the compounds on MCF-7 cells treated for 96 h. FIG. 4E is a graph depicting experimental data of the effects of the compounds on MEF cells treated for 48 h. Non-linear graphs are derived from triplicate values of two independent experiments and presented as Mean±SD.

FIG. 5 is a series of graphs depicting experimental data from breast cancer cells treated with indicated amount of Ospemifene and compounds 6 and 7 for 24 h and apoptosis was determined by Live/Dead assay through flow cytometry.

FIG. 6 is an image of the overlay of the conformations of reference compound (tetrahydroisochiolin) obtained from the X-ray structure (in red) and present docking study (in green).

FIG. 7, comprising FIGS. 7A-7B, depicts the conformations of compound 7 and Ospemifene docked into the binding cavity of ERβ. FIG. 7A is an image of compound 7. FIG. 7B is an image of Ospemifene. Only those amino acid residues of ERβ showing important interactions with ligands are visualized, in lines format. Compounds are depicted as blue sticks. Hydrogen bonds are shown as green dotted lines, whereas hydrophobic interactions (pep, cationep) are shown as purple dotted lines.

FIG. 8, comprising FIGS. 8A-8D, depicts a series of images of conformations of Tamoxifen and compounds of the invention. FIG. 8A is an image of the conformation of 6 (in blue sticks) docked into the binding cavity of ERβ. FIG. 8B is an image of the conformation of 8 (in blue sticks) docked into the binding cavity of ERβ. FIG. 8C is an image of the conformation of Tamoxifen (in blue sticks) docked into the binding cavity of ERβ. FIG. 8D is an image of the conformation of 2 (in blue sticks) docked into the binding cavity of ERβ. Only those amino acid residues of ERβ showing important interactions with ligands are visualized, in lines format. Hydrogen bonds are shown as green dotted lines, whereas hydrophobic interactions are shown as light pink dotted lines.

FIG. 9, comprising FIGS. 9A-9B, depicts a series of images of conformations of compound 7. FIG. 9A is an image of the docked conformation of 7 (blue sticks) showing important interactions with the binding cavity of ERα. Only interacting amino acid residues, in lines format, are shown. Hydrophobic interactions are shown as purple dotted lines. FIG. 9B is an image of the surface representation of ERα showing deep penetration of 7 into its binding pocket.

FIG. 10 is a ¹H NMR spectrum of compound 2 in MeOD.

FIG. 11 is a ¹H NMR spectrum of compound 4 in CDCl₃.

FIG. 12 is a ¹H NMR spectrum of compound 5 in CDCl₃.

FIG. 13 is a ¹H NMR spectrum of compound 8 in MeOD.

FIG. 14 is a ¹³C NMR spectrum of compound 2 in MeOD.

FIG. 15 is a ¹³C NMR spectrum of compound 4 in CDCl₃.

FIG. 16 is a ¹³C NMR spectrum of compound 5 in CDCl₃.

FIG. 17 is a ¹³C NMR spectrum of compound 8 in MeOD.

FIG. 18 is a scheme of an exemplary synthesis of compounds of the invention.

FIG. 19 is an image of the structure of compound 23 illustrating different protons.

FIG. 20, comprising FIGS. 20A-20D, depicts experimental data demonstrating the effects of Ospemifene, Tamoxifen and compounds 13, 22, 23 and 25 on breast cancer cells survival. Breast cancer cells (MDA-MB-231 and MCF-7) were treated with increasing concentrations of selected compounds along with Ospemifene and Tamoxifen (0.5-100 μM) for 72 h. Cell viability was then analyzed by the MTT assay. FIG. 20A is a graph of experimental data from MCF-7 cells treated with Ospemifene, Tamoxifen and compounds 13, 22, 23 and 25. FIG. 20B is a graph of experimental data from MDA-MB-231 cells treated with Ospemifene, Tamoxifen and compounds 13, 22, 23 and 25. FIG. 20C is a graph of experimental data from MCF-7 cells treated with Ospemifene, Tamoxifen and compound 13. FIG. 20D is a graph of experimental data from MDA-MB-231 cells treated with Ospemifene, Tamoxifen and compound 13. Non-linear graphs are derived from triplicate values of two independent experiments and presented as Mean±SD.

FIG. 21, comprising FIGS. 21A-21B, depicts experimental data demonstrating the effects of compounds of the invention on the expression of proteins associated with adhesion, migration and metastasis. FIG. 21A is an image of an immunoblot of MDA-MB-231 cells treated with compounds of the invention. FIG. 21B is an image of an immunoblot of MCF-7 cells treated with compounds of the invention. Breast cancer cells (2×10⁶) were treated with the indicated concentrations of compounds for 24 h, after which Western blotting was performed.

FIG. 22, comprising FIGS. 22A-22B, depicts experimental data demonstrating that compounds of the invention inhibit migration of ER-negative breast cancer cells. FIG. 22A is a series of images depicting scratch assays of MDA-MB-231 cells. Cells were treated with compounds or DMSO (vehicle) and then monolayers were wounded and migration was allowed to proceed for 24 h and migration was measured. FIG. 22B is a graph of experimental data of percent migration relative to control treated cells determined by performing scratch assays in MDA-MB-231 cells.

FIG. 23, comprising FIGS. 23A-23B, depicts experimental data demonstrating that compound 13 inhibits invasion of ER-negative breast cancer cells. FIG. 23A is a series of images depicting MDA-MB-231 cells treated with DMSO or the indicated concentrations of compound 13 and allowed to invade through Matrigel coated membranes (8.0 μm) for 24 h. FIG. 23B is a graph of calculated icell invasion.

FIG. 24, comprising FIGS. 24A-24B, depicts images of native inhibitors (estradiol and genistein) in the binding site of both proteins (ERα and ERβ). FIG. 24A is an images of the overlay of the poses of native ligand (17β-estradiol) obtained from the X-ray structure (in red) and current docking study (in green) for ERα. FIG. 24B is an image of the overlay of the poses of native ligand (genistein) obtained from the X-ray structure (in red) and present docking study (in green) for ERβ.

FIG. 25, comprising FIGS. 25A-25B, depicts a docked complex of compounds 13 and 25 with ERα. FIG. 25A is an image of a docked complex of compound 13 with ERα. FIG. 25B is an image of a docked complex of compound 25 with ERα. Only interacting amino acid residues of the protein are shown (in blue lines format). Ligands are shown in sticks format (lemon color). Hydrogen bonds are shown as green dotted lines, non-conventional hydrogen bond as magenta dotted lines and hydrophobic interactions are shown as red dotted lines.

FIG. 26, comprising FIGS. 26A-26B, depicts a docked complex of compounds 13 and 25 with ERβ. FIG. 26A is an image of a docked complex of compound 13 with ERβ. FIG. 26B is an image of a docked complex of compound 25 with ERβ. Only interacting amino acid residues of the protein are shown (in blue lines format). Ligands are shown in sticks format (lemon color). Hydrogen bonds are shown as green dotted lines, non-conventional hydrogen bond as magenta dotted lines and hydrophobic interactions are shown as red dotted lines.

FIG. 27 is an image of examples of Reported Estrogens, Partial Antiestrogens (SERMs) and Full Antiestrogens.

FIG. 28, comprising FIGS. 28A-28B, depicts experimental data demonstrating that compound 13 is more effective in treating ER-negative (ER−) and ER-positive (ER+) breast cancer cells than Ospemifene and Tamoxifen. FIG. 28A is a graph of experimental data of ER-negative (MDA-MB-231) breast cancer cells treated with increasing amounts of compound 13 along with Ospemifene and Tamoxifen (0.5-100 μM) for 48 h. FIG. 28B is a graph of experimental data of ER+ (MCF-7) breast cancer cells treated with increasing amounts of compound 13 along with Ospemifene and Tamoxifen (0.5-100 μM) for 48 h. Cell viability was then analyzed by the MTT assay. Cell viability is presented as non-linear regression plot.

### DETAILED DESCRIPTION OF THE INVENTION

This invention includes the unexpected identification of novel triarylethylene compounds that are useful for the treatment of cancer. As demonstrated herein, the compounds of the present invention have been shown to be effective chemotherapeutic agents for the treatment of breast cancer.

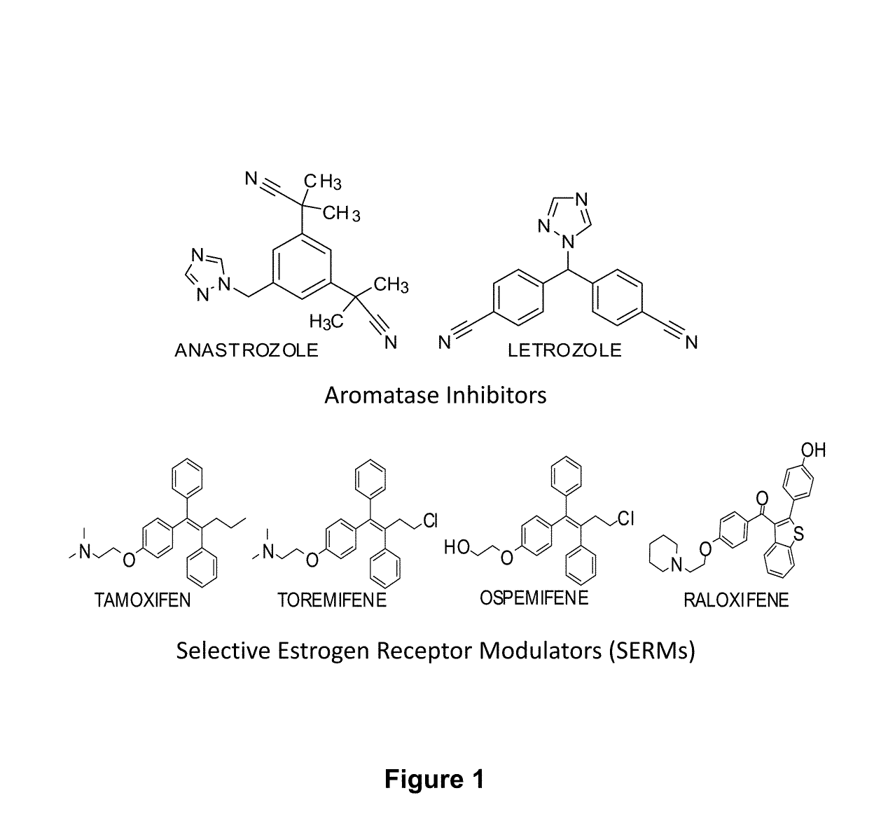

The compounds of the present invention provide improvements over other cancer therapeutics known in the prior art. In one embodiment, compounds of the invention are more potent than known Selective Estrogen Receptor Modulators (SERMs) such as Ospemifene and Tamoxifen (FIG. 1). SERMs are most commonly used in the clinic to treat breast cancer patients and are found to be effective only against ER+ breast cancer. As demonstrated herein, compounds of the invention showed efficacy against both ER+ and ER− breast cancer, and may be useful for a wider array of patients than conventional SERMs. Docking studies performed against estrogen receptors ERα and ERβ demonstrated that the compounds of the invention exhibited stronger binding affinities with both ERα and ERβ compared to Ospemifene and Tamoxifen. Moreover, the compounds of the invention may also be useful in the treatment of patients that suffer from tumors, such as chemotherapeutic resistant tumors.

The present invention also includes novel methods of treating or preventing cancer using the compounds of the invention. In one embodiment, the cancer is selected from the group consisting of lung cancer, colon cancer, melanoma, breast cancer, ovarian cancer, prostate cancer, liver cancer, pancreatic cancer, CNS tumors (including brain tumors), neuroblastoma, leukemia, bone cancer, intestinal cancer, lymphoma, and combinations thereof. In one embodiment, the cancer is breast cancer.

The present invention includes a composition comprising at least one compound of the invention, wherein the composition optionally further comprise at least one additional therapeutic agent. In one embodiment, the additional therapeutic agent is a chemotherapeutic agent.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal,” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a sign or symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the terms “effective amount,” “pharmaceutically effective amount” and “therapeutically effective amount” refer to a nontoxic but sufficient amount of an agent to provide the desired biological result. That result may be reduction and/or alleviation of a sign, a symptom, or a cause of a disease or disorder, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

An “effective amount” of a delivery vehicle is that amount sufficient to effectively bind or deliver a compound.

As used herein, the term “potency” refers to the dose needed to produce half the maximal response (ED₅₀).

As used herein, the term “efficacy” refers to the maximal effect (Eₘₐₓ) achieved within an assay.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following

moieties:

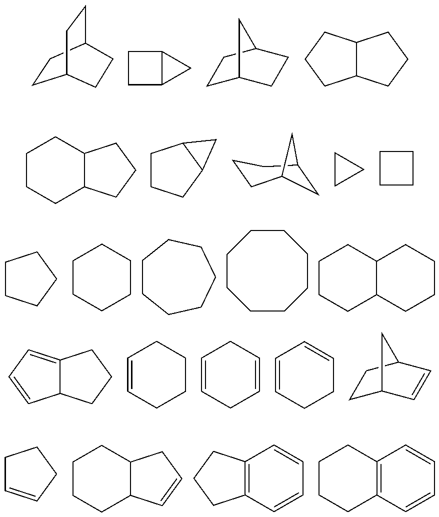

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heterocycloalkyl” or “heterocyclyl” refers to a heteroalicyclic group containing one to four ring heteroatoms each selected from O, Sand N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

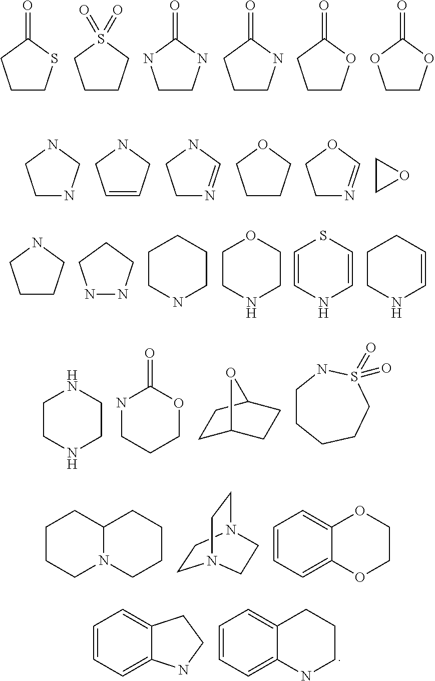

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “aryl-(C₁-C₃)alkyl” means a functional group wherein a one- to three-carbon alkylene chain is attached to an aryl group, e.g., —CH₂CH₂-phenyl. Preferred is aryl-CH₂- and aryl-CH(CH₃)—. The term “substituted aryl-(C₁-C₃)alkyl” means an aryl-(C₁-C₃)alkyl functional group in which the aryl group is substituted. Similarly, the term “heteroaryl-(C₁-C₃)alkyl” means a functional group wherein a one to three carbon alkylene chain is attached to a heteroaryl group, e.g., —CH₂CH₂-pyridyl. The term “substituted heteroaryl-(C₁-C₃)alkyl” means a heteroaryl-(C₁-C₃)alkyl functional group in which the heteroaryl group is substituted.

As used herein, the term “heteroaryl” or “heteroaromatic” refers to a heterocycle having aromatic character. A polycyclic heteroaryl may include one or more rings that are partially saturated. Examples include the following moieties:

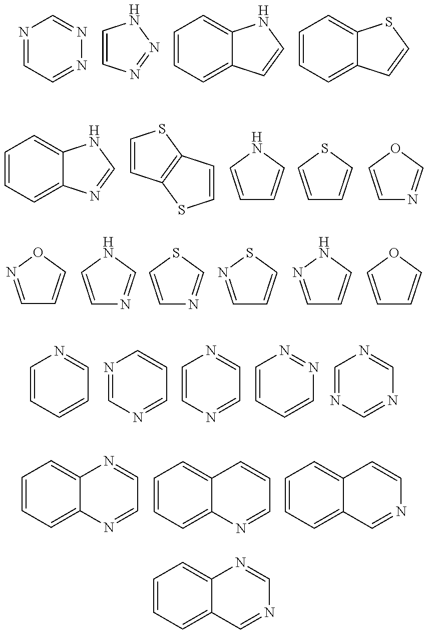

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl. Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

As used herein, the term “Ospemifene” refers to (Z)-2-(4-(4-Chloro-1,2-diphenylbut-1-enyl)phenoxy)ethanol.

As used herein, the term “Tamoxifen” refers to (Z)-2-[4-(1,2-diphenylbut-1-enyl)phenoxy]-N,N-dimethylethanamine.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Compounds Useful within the Invention

The compounds of the present invention may be synthesized using techniques well-known in the art of organic synthesis. The starting materials and intermediates required for the synthesis may be obtained from commercial sources or synthesized according to methods known to those skilled in the art.

In one aspect, the compound of the invention is a compound of formula (I), or a salt or solvate thereof:

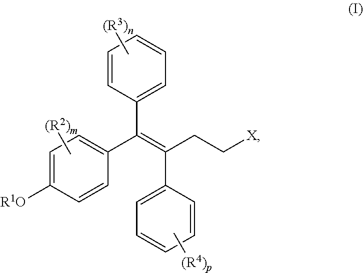

wherein in formula (I):

R¹ is selected from the group consisting of H and alkyl, wherein the alkyl group is optionally substituted;

each occurrence of R², R³, and R⁴ is independently selected from the group consisting of H, —C₁-C₆ alkyl, —C₁-C₆ fluoroalkyl, —C₁-C₆ heteroalkyl, F, Cl, Br, I, —CN, —NO₂, —OR⁴, —SR⁴, —S(═O)R⁴, —S(═O)₂R⁴, —NHS(═O)₂R⁴, —C(NH)(NH₂), —C(═O)R⁴, —OC(═O)R⁴, —CO₂R⁴, —OCO₂R⁴, —CH(R⁴)₂, —N(R⁴)₂, —C(═O)N(R⁴)₂, —OC(═O)N(R⁴)₂, —NHC(═O)NH(R⁴), —NHC(═O)R⁴, —NHC(═O)OR⁴, —C(OH)(R⁴)₂, and —C(NH₂)(R⁴)₂;

X is selected from the group consisting of N₃, N(R⁵)(R⁶), Cl, Br, I, and F;

R⁵ and R⁶ are each independently selected from the group consisting of H, —C₁-C₆ alkyl, —C(O)R⁷, and —C(S)R⁷;

R⁷ is selected from the group consisting of OR⁸, N(R⁸)(R⁹), C(O)R⁸, and C(O)N(R⁸)(R⁹);

R⁸ and R⁹ are each independently selected from the group consisting of hydrogen, —C₁-C₆ alkyl, aryl, cycloalkyl, and —C₁-C₆ alkyl-aryl, wherein the alkyl, aryl, cycloalkyl, or alkylaryl group may be optionally substituted, and wherein R⁸ and R⁹ may combine to form a ring, wherein the ring may optionally contain two or more heteroatoms;

m is an integer from 0 to 4;

n is an integer from 0 to 5; and

p is an integer from 0 to 5.

In one embodiment, R¹ is —C₁-C₆ alkyl, wherein the alkyl group is substituted with at least one group selected from the group consisting of —OH, alkoxy, —NH₂, amino, azido, and mesyl, wherein the hydroxy, azido, or amino group is optionally substituted. In one embodiment, R¹ is —C₂ alkyl, wherein the alkyl group is substituted with at least one group selected from the group consisting of —OH, alkoxy, —NH₂, amino, azido, and mesyl, wherein the hydroxy, azido, or amino group is optionally substituted. In one embodiment, R¹ is selected from the group consisting of H, methyl, —(CH₂)₂OH, —(CH₂)₂OS(O)₂CH₃, —(CH₂)₂N₃, —(CH₂)₂NH₂, and —(CH₂)₂N(CH₃)₂. In another embodiment, R¹ is selected from the group consisting of methyl, —(CH₂)₂OH, and —(CH₂)₂NH₂. In another embodiment, R¹ is methyl.

In one embodiment, X is N(R⁵)(R⁶). In one embodiment, R⁵ and R⁶ are each H. In another embodiment, R⁵ is H and R⁶ is —C(O)R⁷. In another embodiment, either R⁵ and R⁶ are each H or R⁵ is H and R⁶ is —C(O)R⁷.

In one embodiment, R⁷ is selected from the group consisting of OR⁸, C(O)R⁸, and C(O)N(R⁸)(R⁹). In another embodiment, R⁷ is OR⁸. In another embodiment, R⁷ is C(O)R⁸. In another embodiment, R⁷ is C(O)N(R⁸)(R⁹).

In one embodiment, R⁸ is aryl. In another embodiment, R⁸ is H and R⁹ is selected from the group consisting of cycloalkyl and —C₁-C₆ alkyl-aryl.

In one embodiment, m is 0. In another embodiment, n is 0. In another embodiment, p is 0. In another embodiment, m is 0, n is 0, and p is 0.

In one embodiment, the compound of the invention is selected from the group consisting of:

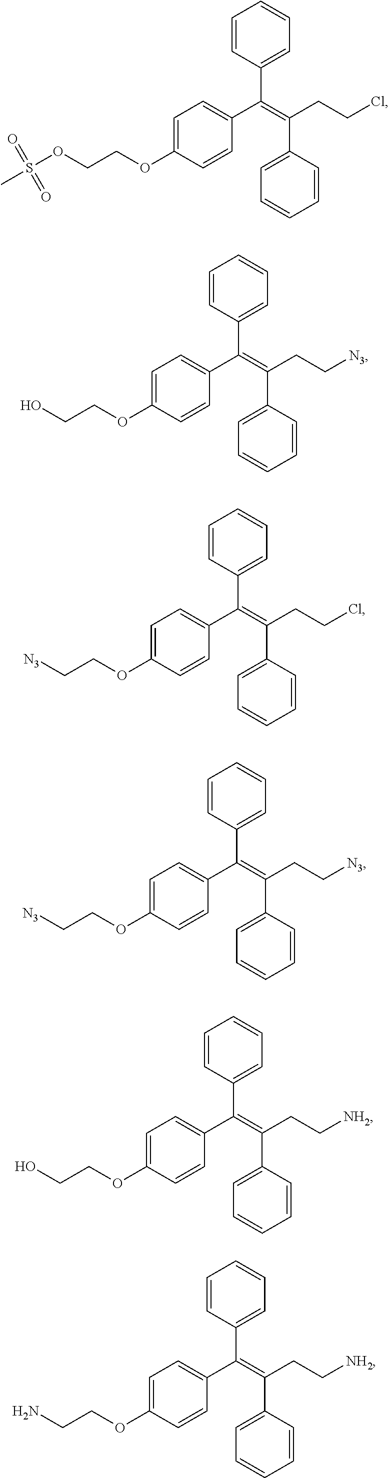

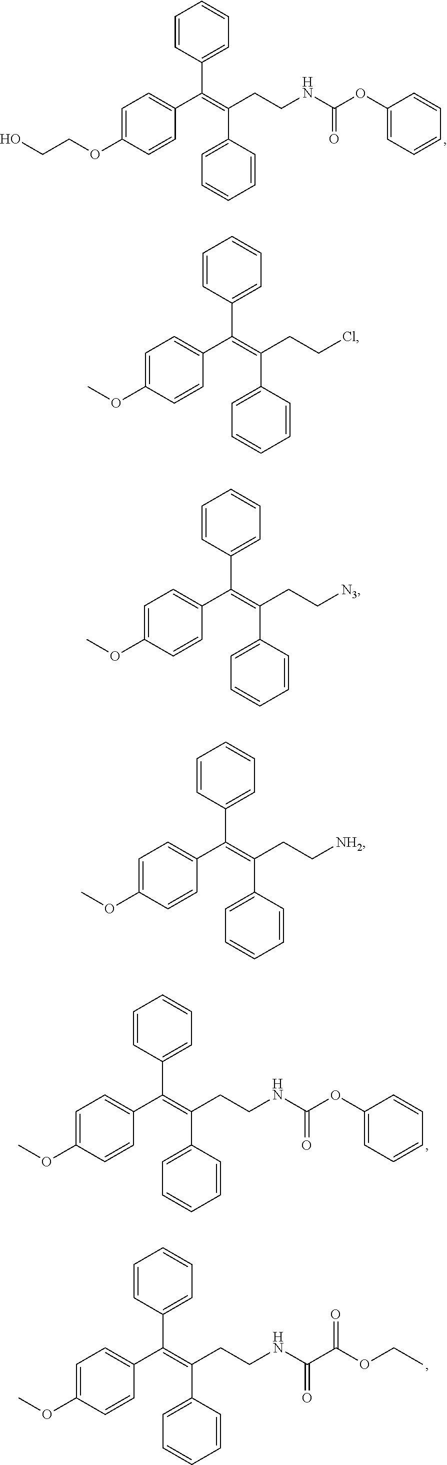

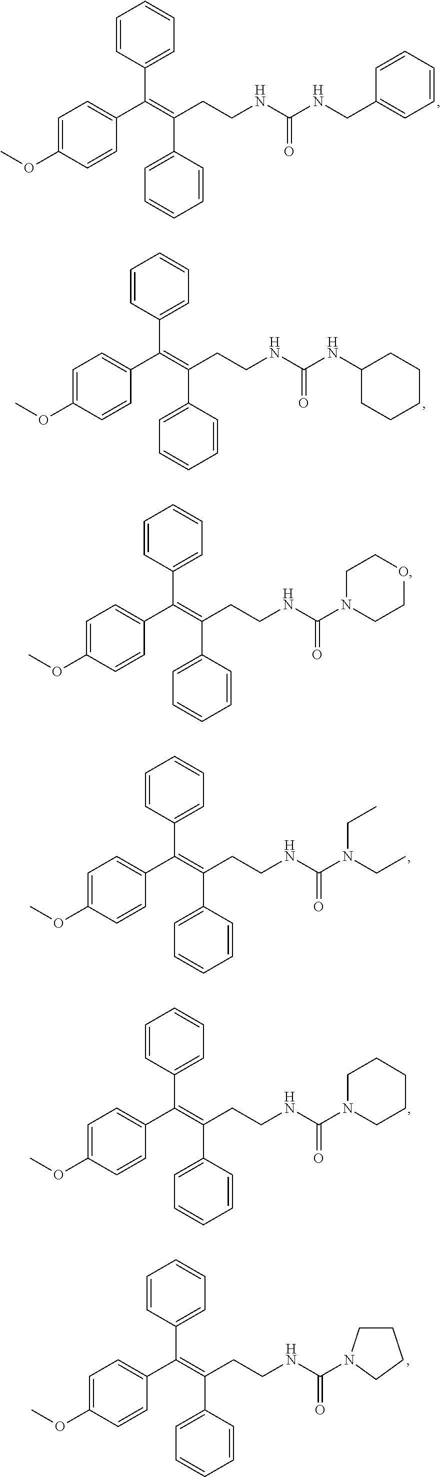

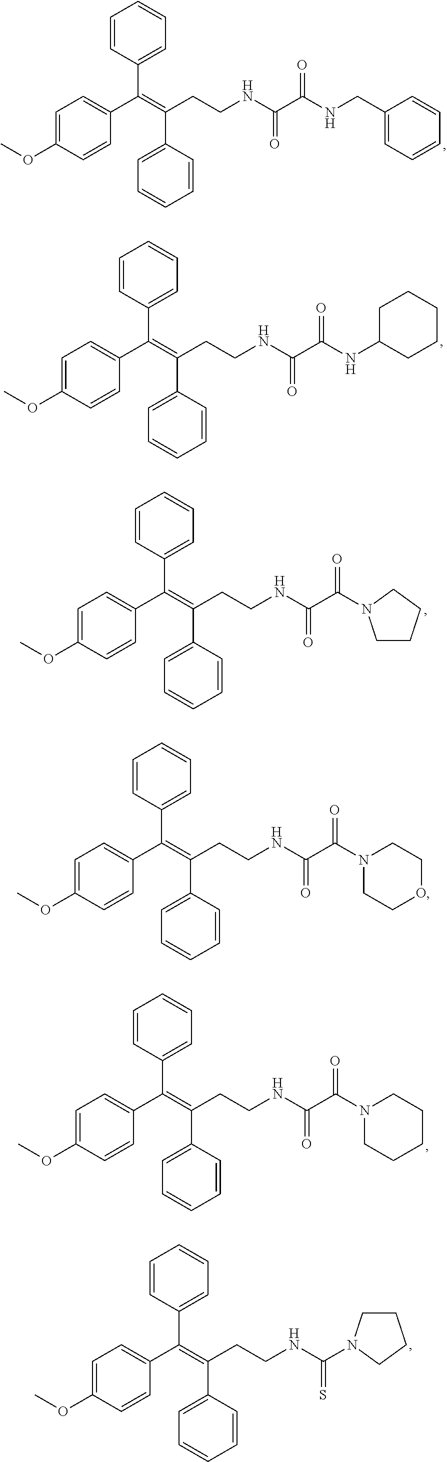

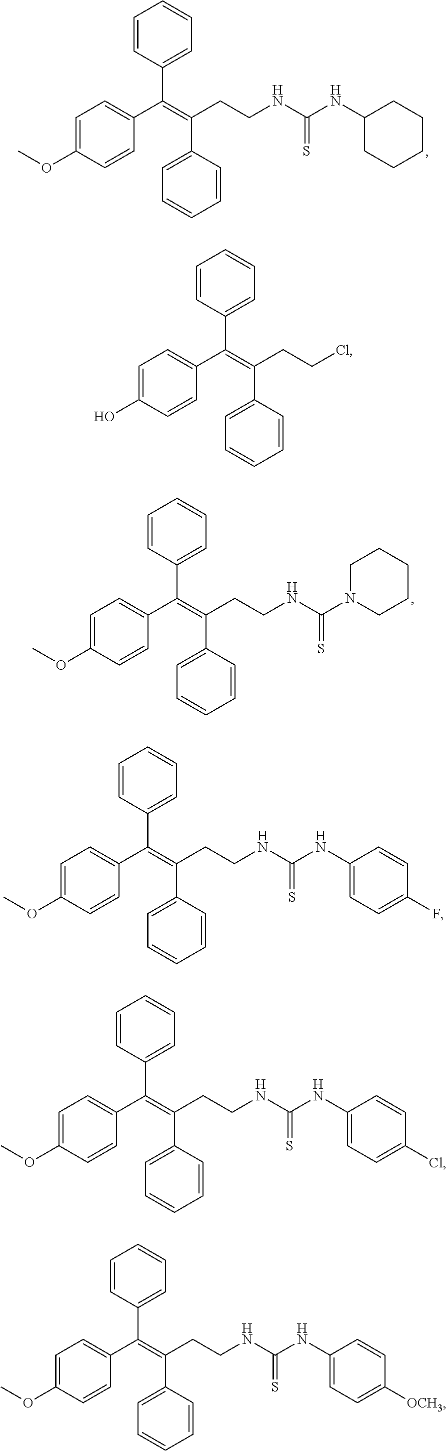

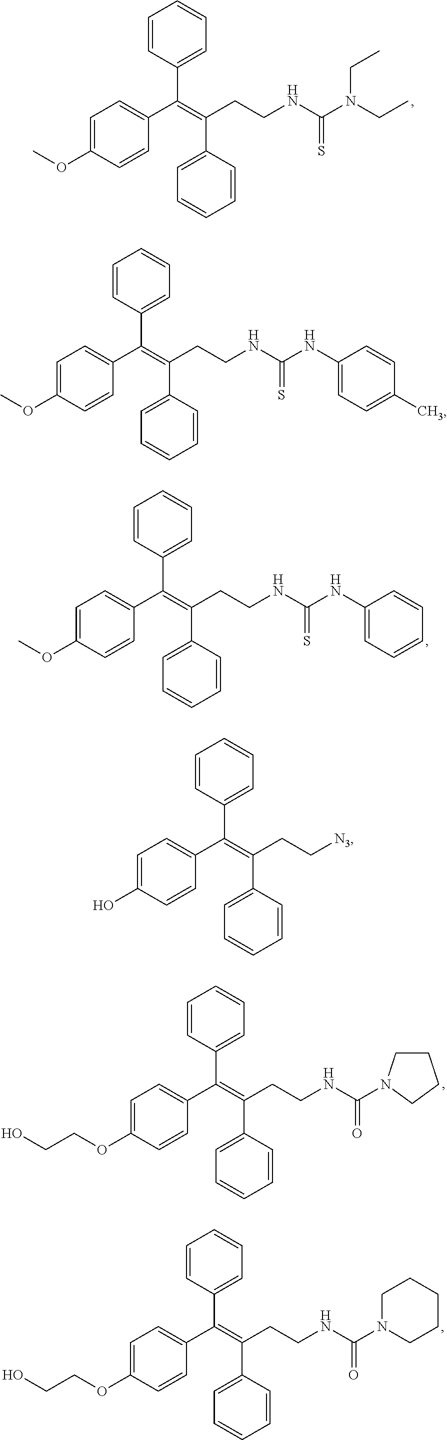

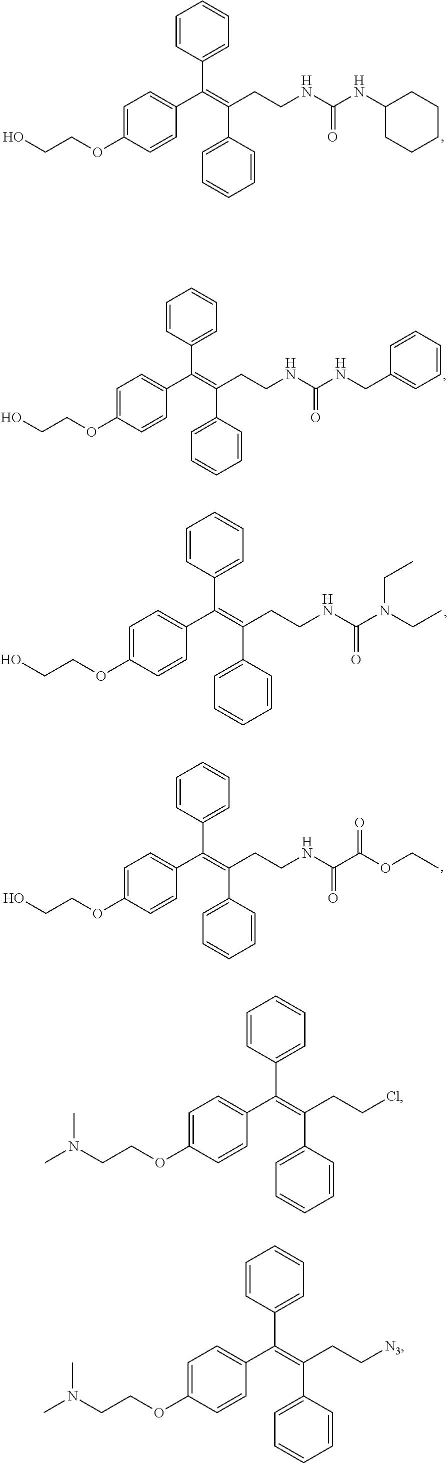

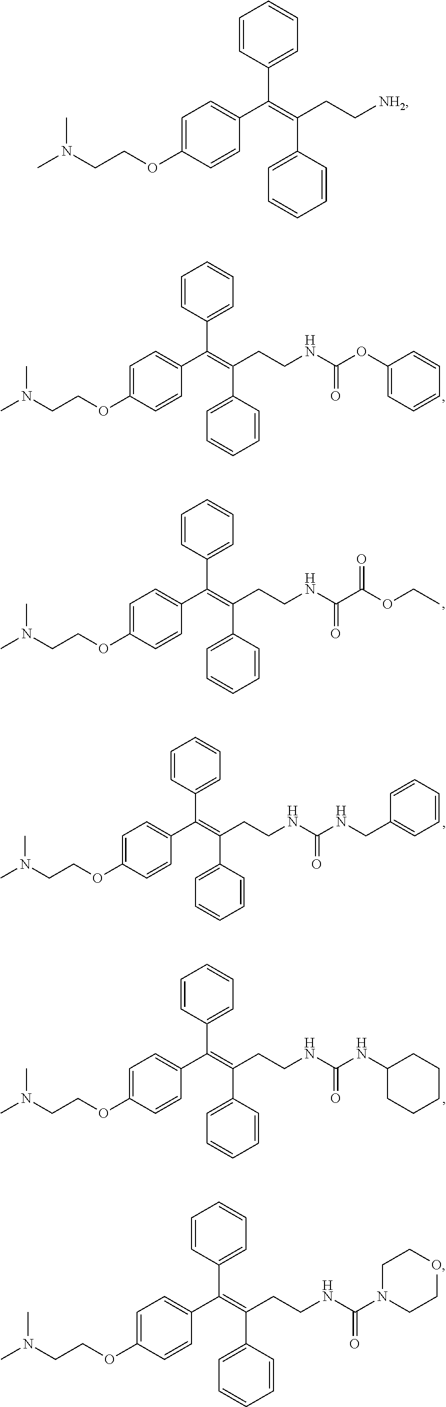

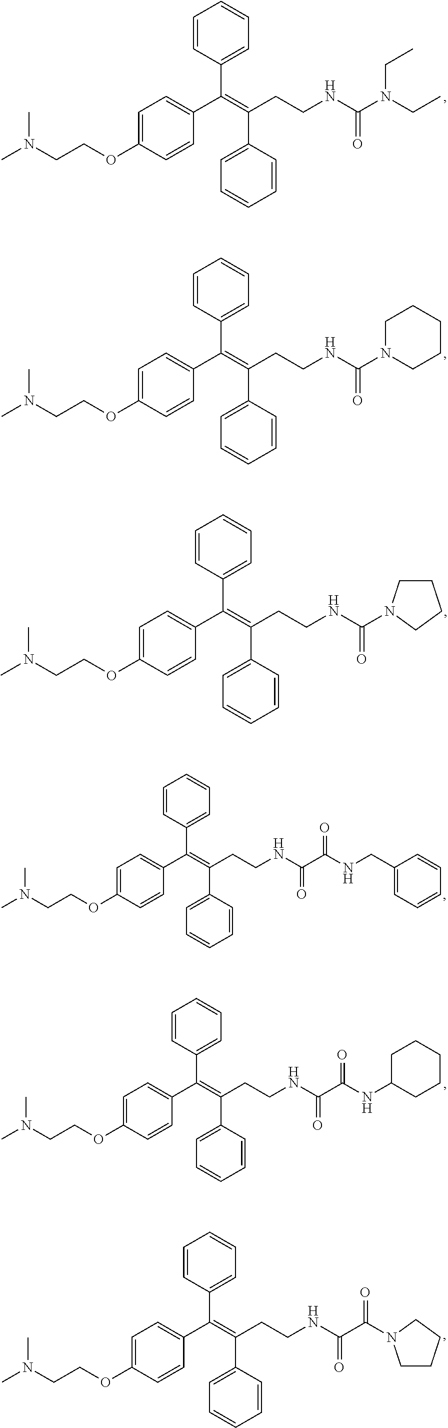

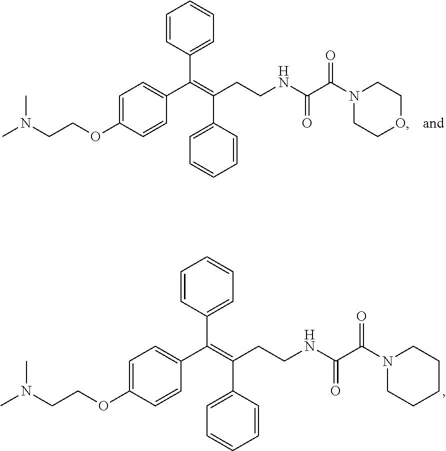 [SMILES: CN(C)CCOc1ccc(/C(=C(/CCNC(=O)C(=O)N2CCOCC2)c2ccccc2)c2ccccc2)cc1 ; CN(C)CCOc1ccc(/C(=C(/CCNC(=O)C(=O)N2CCCCC2)c2ccccc2)c2ccccc2)cc1]

a salt or solvate thereof, and any combinations thereof.

In one embodiment, the compound is selected from the group consisting of

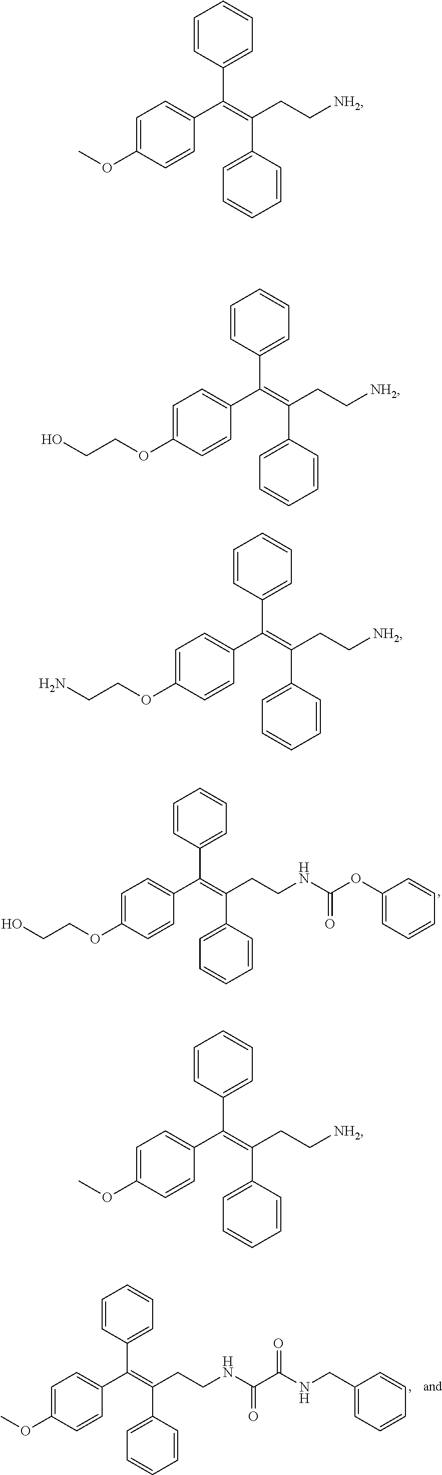

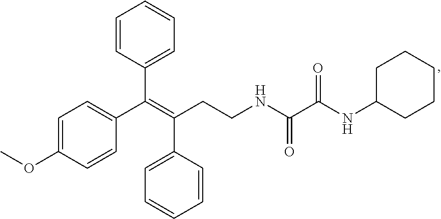 [SMILES: COc1ccc(/C(=C(/CCNC(=O)C(=O)NC2CCCCC2)c2ccccc2)c2ccccc2)cc1]

a salt or solvate thereof, and any combinations thereof.

In one embodiment, there is the proviso that the compound of formula (I) is not

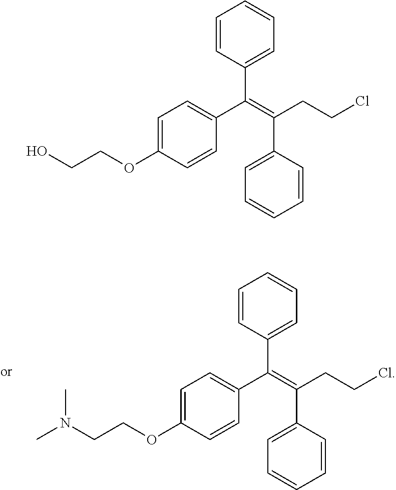 [SMILES: OCCOc1ccc(/C(=C(/CCCl)c2ccccc2)c2ccccc2)cc1 ; "or" ; CN(C)CCOc1ccc(/C(=C(/CCCl)c2ccccc2)c2ccccc2)cc1]

### Preparation of the Compounds of the Invention

Compounds of formula (I) may be prepared by the general schemes described herein, using the synthetic method known by those skilled in the art. The following examples illustrate non-limiting embodiments of the invention.

In one aspect, compounds useful in the invention are synthesized by the reaction of a benzophenone with a benzyl ketone comprised of an alkyl halide. In a non-limiting embodiment, the benzophenone and benzyl ketone are treated with TiCl₄ and zinc to produce a triarylethylene compound.

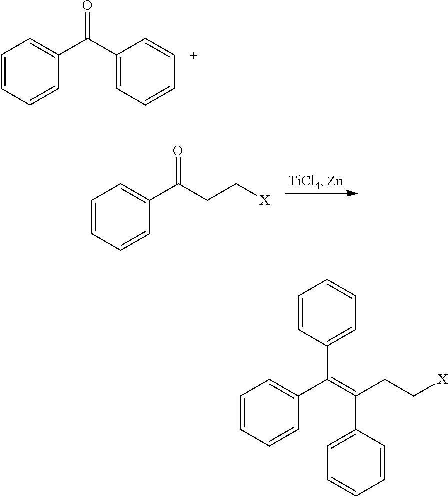

In another non-limiting embodiment, the triarylene compound is treated with sodium azide to form an alkyl azide, which is subsequently reduced using zinc in ammonium chloride to produce the amine that may optionally be acylated with an acyl chloride to produce compounds of the invention.

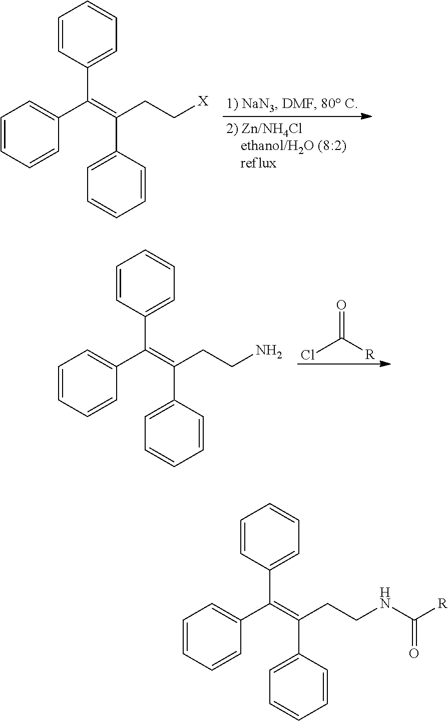

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

In one embodiment, the compounds of the invention may exist as tautomers. All tautomers are included within the scope of the compounds presented herein.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

In one embodiment, sites on, for example, the aromatic ring portion of compounds of the invention are susceptible to various metabolic reactions. Incorporation of appropriate substituents on the aromatic ring structures may reduce, minimize or eliminate this metabolic pathway. In one embodiment, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²F, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as 11C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In one embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In one embodiment, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In one embodiment, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

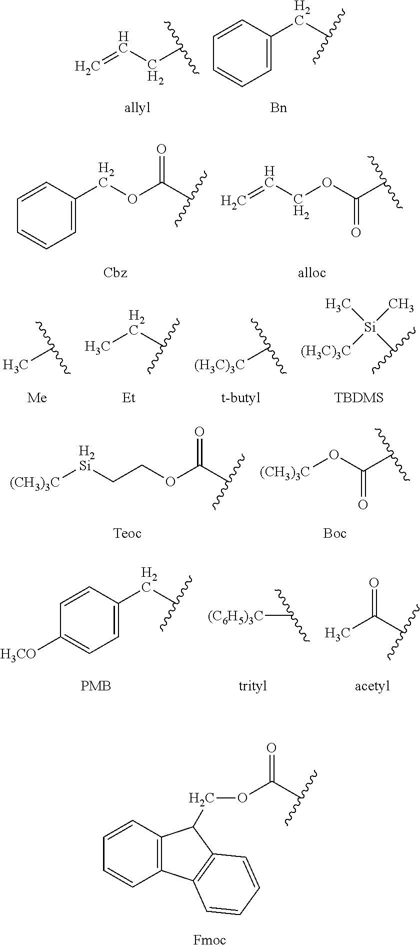

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Methods of the Invention

The invention includes a method of treating or preventing cancer in a subject in need thereof. The method comprises administering to the subject an effective amount of a therapeutic composition comprising a compound of the invention. Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the compositions of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers that can be treated with the compositions of the invention include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, that can be treated with the compositions of the invention, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In one embodiment, the cancer is selected from the group consisting of lung cancer, colon cancer, melanoma, breast cancer, ovarian cancer, prostate cancer, liver cancer, pancreatic cancer, CNS tumors (including brain tumors), neuroblastoma, leukemia, bone cancer, intestinal cancer, lymphoma, and combinations thereof. In one embodiment, the cancer is breast cancer. In one embodiment, the method further comprises administering to the subject an additional therapeutic agent.

In one embodiment, administering the compound of the invention to the subject allows for administering a lower dose of the therapeutic agent compared to the dose of the therapeutic agent alone that is required to achieve similar results in treating or preventing cancer in the subject. For example, in one embodiment, the compound of the invention enhances the anti-cancer activity of the additional therapeutic compound, thereby allowing for a lower dose of the therapeutic compound to provide the same effect.

In one embodiment, the compound of the invention and the therapeutic agent are co-administered to the subject. In another embodiment, the compound of the invention and the therapeutic agent are coformulated and co-administered to the subject.

In one embodiment, the subject is a mammal. In another embodiment, the mammal is a human.

### Combination Therapies

The compounds of the present invention are intended to be useful in combination with one or more additional compounds. In certain embodiments, these additional compounds may comprise compounds of the present invention or therapeutic agents known to treat or reduce the symptoms or effects of cancer. Such compounds include, but are not limited to, chemotherapeutics and the like.

In non-limiting examples, the compounds of the invention may be used in combination with one or more therapeutic agents (or a salt, solvate or prodrug thereof).

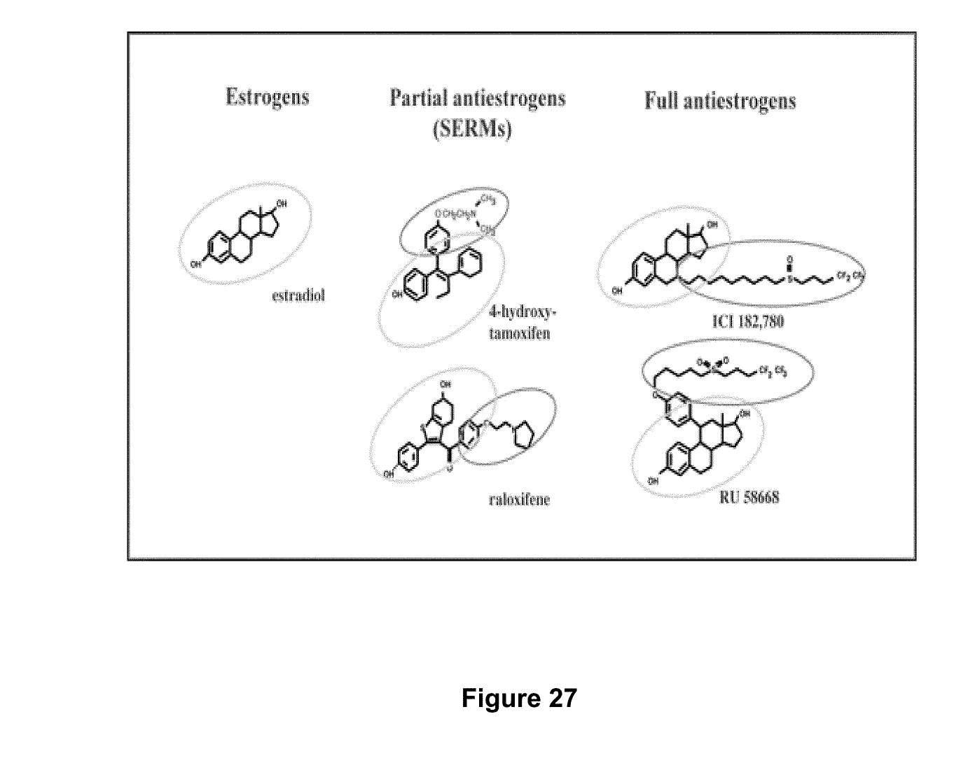

In certain embodiments, the compound of the invention may be administered to a subject in conjunction with (e.g. before, simultaneously, or following) any number of relevant treatment modalities including chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the compounds of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the compounds of the present invention are administered following B-cell ablative therapy such as agents that react with CD20, e.g., Rituxan. In another embodiment, the compounds of the present invention are administered in conjunction with Ospemifene, Tamoxifen, Raloxifene, or other drugs such as ICI 182,780 and RU 58668. Tamoxifen and Raloxifene may act as partial antiestrogens, and the drugs such as ICI 182,780 and RU 58668 (FIG. 27) may act as full antiestrogens. In another embodiment, the compounds of the invention are administered in conjunction with aromatase inhibitors. Non-limiting examples of aromatase inhibitors include Exemestane, Letrozole, and Anastrozole.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either before or after the onset of cancer. Further, several divided dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, (e.g., human), may be carried out using known procedures, at dosages and for periods of time effective to treat cancer in the patient. An effective amount of the therapeutic compound necessary to achieve a therapeutic effect may vary according to factors such as the state of the disease or disorder in the patient; the age, sex, and weight of the patient; and the ability of the therapeutic compound to treat a cancer in the patient. Dosage regimens may be adjusted to provide the optimum therapeutic response. For example, several divided doses may be administered daily. In another example, the dose may be proportionally reduced as indicated by the exigencies of the therapeutic situation. A non-limiting example of an effective dose range for a therapeutic compound of the invention is from about 1 mg/kg to about 5,000 mg/kg of body weight/per day. One of ordinary skill in the art would be able to assess the relevant factors and make the determination regarding the effective amount of the therapeutic compound without undue experimentation.

Actual dosage levels of the active ingredients in the pharmaceutical compositions of this invention may be varied to obtain an amount of the active ingredient that is effective to achieve the desired therapeutic response for a particular patient, composition, and mode of administration, without generating excessive side effects in the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical professional, e.g., physician or veterinarian, having ordinary skill in the art may readily determine and prescribe the effective amount of the pharmaceutical composition required. For example, the physician or veterinarian could start with a dosage of the compound of the invention in the pharmaceutical composition at a level that is lower than the level required to achieve the desired therapeutic effect, and then increase the dosage over time until the desired effect is achieved.

In particular embodiments, it is advantageous to formulate the compound in dosage unit form for ease of administration and uniformity of dosage. “Dosage unit form” as used herein refers to a physically discrete unit containing a predetermined quantity of therapeutic compound calculated to produce the desired therapeutic effect, in association with the required pharmaceutical vehicle. The dosage unit forms of the invention can be selected based upon (a) the unique characteristics of the therapeutic compound and the particular therapeutic effect to be achieved, and (b) the limitations inherent in the art of compounding/formulating such a therapeutic compound for the treatment of cancer in a patient.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), vegetable oils, and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments, it is useful to include isotonic agents, for example, sugars, sodium chloride, or polyalcohols such as mannitol and sorbitol, in the composition. Prolonged absorption of the injectable compositions can be achieved by including in the composition an agent which delays absorption, for example, aluminum monostearate or gelatin. In one embodiment, the pharmaceutically acceptable carrier is DMSO, alone or in combination with other carriers.

The therapeutically effective amount or dose of a compound of the present invention depends on the age, sex and weight of the patient, the current medical condition of the patient and the severity of the cancer in the patient being treated. The skilled artisan is able to determine appropriate doses depending on these and other factors.

The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

Doses of the compound of the invention for administration may be in the range of from about 1 μg to about 10,000 mg, from about 20 μg to about 9,500 mg, from about 40 μg to about 9,000 mg, from about 75 μg to about 8,500 mg, from about 150 μg to about 7,500 mg, from about 200 μg to about 7,000 mg, from about 3050 μg to about 6,000 mg, from about 500 μg to about 5,000 mg, from about 750 μg to about 4,000 mg, from about 1 mg to about 3,000 mg, from about 10 mg to about 2,500 mg, from about 20 mg to about 2,000 mg, from about 25 mg to about 1,500 mg, from about 30 mg to about 1,000 mg, from about 40 mg to about 900 mg, from about 50 mg to about 800 mg, from about 60 mg to about 750 mg, from about 70 mg to about 600 mg, from about 80 mg to about 500 mg, and any and all whole or partial increments therebetween.

In some embodiments, the dose of a compound of the invention is from about 1 mg to about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, the dosage of a second compound as described elsewhere herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

In one embodiment, the compositions of the invention are administered to the patient from about one to about five times per day or more. In various embodiments, the compositions of the invention are administered to the patient, 1-7 times per day, 1-7 times every two days, 1-7 times every 3 days, 1-7 times every week, 1-7 times every two weeks, and 1-7 times per month. It is readily apparent to one skilled in the art that the frequency of administration of the various combination compositions of the invention will vary from individual to individual depending on many factors including, but not limited to, age, the disease or disorder to be treated, the severity of the disease or disorder to be treated, gender, overall health, and other factors. Thus, the invention should not be construed to be limited to any particular dosing regime and the precise dosage and composition to be administered to any patient is determined by the medical professional taking all other factors about the patient into account.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, may be reduced to a level at which the improved disease is retained. In some embodiments, a patient may require intermittent treatment on a long-term basis, or upon any recurrence of the disease or disorder.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the present invention is directed to a packaged pharmaceutical composition comprising a container holding a therapeutically effective amount of a compound of the invention, alone or in combination with a second pharmaceutical agent; and instructions for using the compound to treat or prevent cancer in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, suitable forms include tablets, dragees, liquids, drops, suppositories, or capsules, caplets and gelcaps. The compositions formulated for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation involves the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of G-protein receptor-related diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release refers to a drug formulation that provides for gradual release of a drug over an extended period of time, and that may, although not necessarily, result in substantially constant blood levels of a drug over an extended time period. The period of time may be as long as a day, a week, or a month or more and should be a release which is longer that the same amount of agent administered in bolus form. The term delayed release is used herein in its conventional sense to refer to a drug formulation that provides for an initial release of the drug after some delay following drug administration and that mat, although not necessarily, includes a delay of from about 10 minutes up to about 12 hours.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term pulsatile release refers to a drug formulation that provides release of the drug in such a way as to produce pulsed plasma profiles of the drug after drug administration.

The term immediate release refers to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art recognize, or are able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

The following Table A includes compounds referred to in the working examples.

TABLE A

| COMPOUND NUMBER | STRUCTURE |
|---|---|
| 1 | 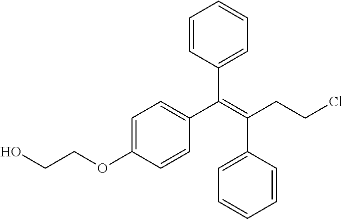 [SMILES: OCCOc1ccc(/C(=C(/CCCl)c2ccccc2)c2ccccc2)cc1] |
| 2 | 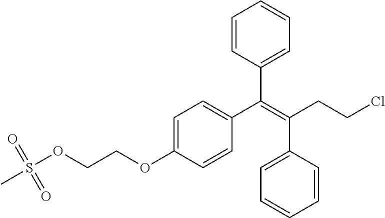 [SMILES: CS(=O)(=O)OCCOc1ccc(/C(=C(/CCCl)c2ccccc2)c2ccccc2)cc1] |
| 3 | 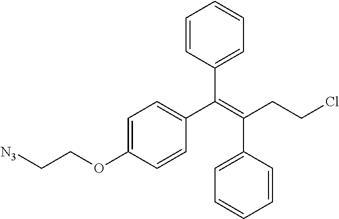 [SMILES: [N-]=[N+]=NCCOc1ccc(/C(=C(/CCCl)c2ccccc2)c2ccccc2)cc1] |
| 4 | 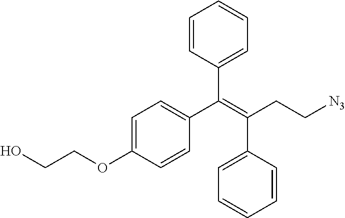 [SMILES: [N-]=[N+]=NCC/C(=C(\c1ccccc1)c1ccc(OCCO)cc1)c1ccccc1] |
| 5 | 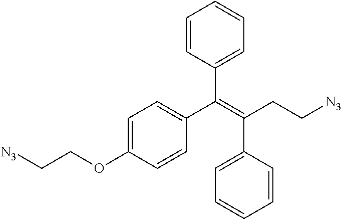 [SMILES: [N-]=[N+]=NCCOc1ccc(/C(=C(/CCN=[N+]=[N-])c2ccccc2)c2ccccc2)cc1] |
| 6 | 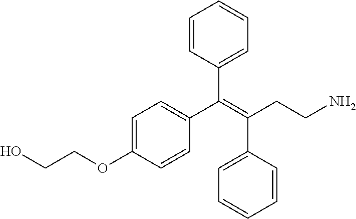 [SMILES: NCC/C(=C(\c1ccccc1)c1ccc(OCCO)cc1)c1ccccc1] |
| 7 | 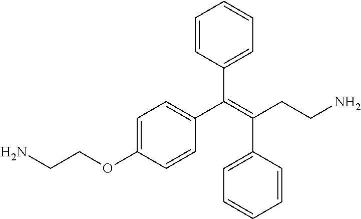 [SMILES: NCCOc1ccc(/C(=C(/CCN)c2ccccc2)c2ccccc2)cc1] |
| 8 | 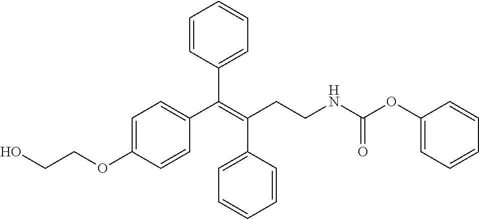 [SMILES: O=C(NCC/C(=C(\c1ccccc1)c1ccc(OCCO)cc1)c1ccccc1)Oc1ccccc1] |
| 11 | 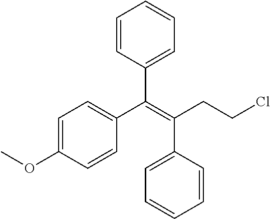 [SMILES: COc1ccc(/C(=C(/CCCl)c2ccccc2)c2ccccc2)cc1] |
| 12 | 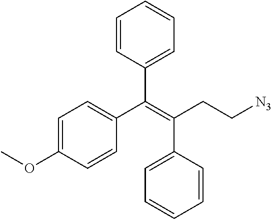 [SMILES: COc1ccc(/C(=C(/CCN=[N+]=[N-])c2ccccc2)c2ccccc2)cc1] |
| 13 | 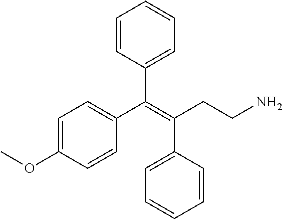 [SMILES: COc1ccc(/C(=C(/CCN)c2ccccc2)c2ccccc2)cc1] |
| 14 | 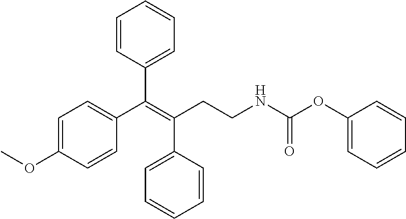 [SMILES: COc1ccc(/C(=C(/CCNC(=O)Oc2ccccc2)c2ccccc2)c2ccccc2)cc1] |
| 15 | 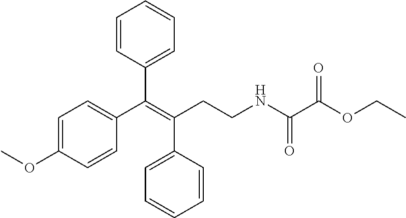 [SMILES: CCOC(=O)C(=O)NCC/C(=C(\c1ccccc1)c1ccc(OC)cc1)c1ccccc1] |
| 16 | 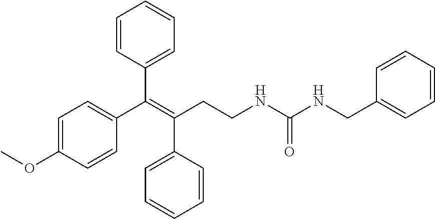 [SMILES: COc1ccc(/C(=C(/CCNC(=O)NCc2ccccc2)c2ccccc2)c2ccccc2)cc1] |
| 17 | 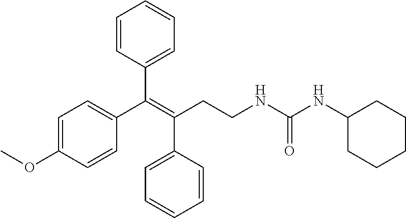 [SMILES: COc1ccc(/C(=C(/CCNC(=O)NC2CCCCC2)c2ccccc2)c2ccccc2)cc1] |
| 18 | 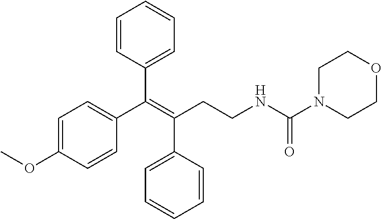 [SMILES: COc1ccc(/C(=C(/CCNC(=O)N2CCOCC2)c2ccccc2)c2ccccc2)cc1] |
| 19 | 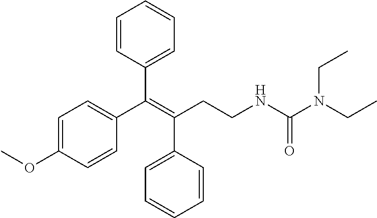 [SMILES: CCN(CC)C(=O)NCC/C(=C(\c1ccccc1)c1ccc(OC)cc1)c1ccccc1] |
| 20 | 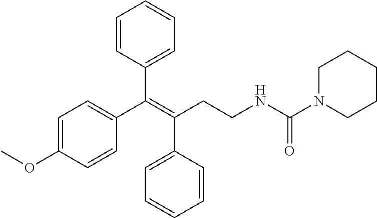 [SMILES: COc1ccc(/C(=C(/CCNC(=O)N2CCCCC2)c2ccccc2)c2ccccc2)cc1] |
| 21 | 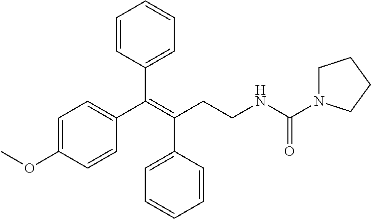 [SMILES: COc1ccc(/C(=C(/CCNC(=O)N2CCCC2)c2ccccc2)c2ccccc2)cc1] |
| 22 | 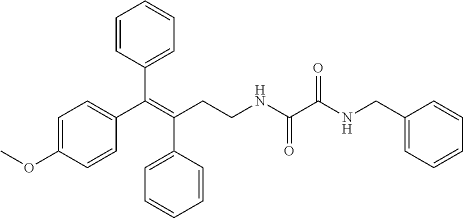 [SMILES: COc1ccc(/C(=C(/CCNC(=O)C(=O)NCc2ccccc2)c2ccccc2)c2ccccc2)cc1] |
| 23 | 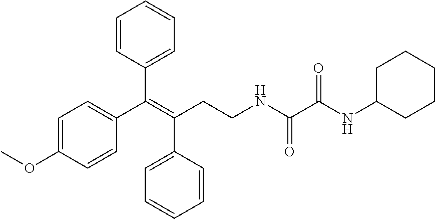 [SMILES: COc1ccc(/C(=C(/CCNC(=O)C(=O)NC2CCCCC2)c2ccccc2)c2ccccc2)cc1] |
| 24 | 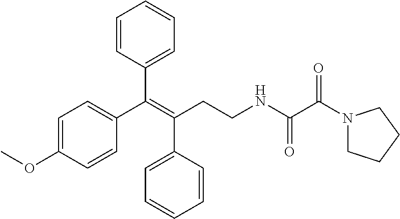 [SMILES: COc1ccc(/C(=C(/CCNC(=O)C(=O)N2CCCC2)c2ccccc2)c2ccccc2)cc1] |
| 25 | 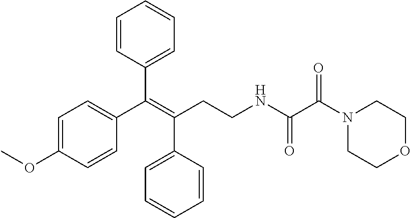 [SMILES: COc1ccc(/C(=C(/CCNC(=O)C(=O)N2CCOCC2)c2ccccc2)c2ccccc2)cc1] |
| 26 | 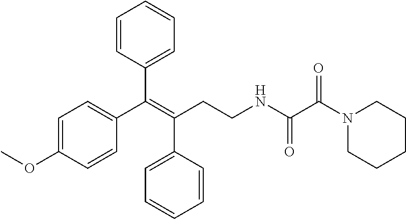 [SMILES: COc1ccc(/C(=C(/CCNC(=O)C(=O)N2CCCCC2)c2ccccc2)c2ccccc2)cc1] |

### Example 1: Design, Synthesis and Evaluation of Ospemifene Analogs as Anti-Breast Cancer Agents

The results described herein demonstrate the synthesis of some novel Ospemifene derived analogs and their evaluation as anti-breast cancer agents against MCF-7 (ER-positive) and MDA-MB-231 (ER-negative) human breast cancer cell lines. Several analogs, for instance, compounds 6, 7 and 8, have been shown to be more effective than recent Selective Estrogen Receptor Modulators (SERMs) i.e. Ospemifene and Tamoxifen, against these cell lines. Compound 8 was relatively more cytotoxic to MCF-7 cells similar to Ospemifene and Tamoxifen, while potent compounds 6 and 7 were equally effective in inhibiting growth of both ER-positive and ER-negative cell lines. The observed activity profiles were further supported by the docking studies performed against estrogen receptors (ERα and ERβ). Compounds 6, 7 and 8 exhibited stronger binding affinities with both ERα and ERβ compared to Ospemifene and Tamoxifen.

The materials and methods employed in these experiments are now described.

### Synthetic Chemistry

### General

Melting points were determined by open capillary using a Veego Programmable Melting/Boiling Point Apparatus and are uncorrected. IR spectra were recorded on Perkin Elmer FT-IR Spectrometer Spectrum Two. Molecular masses and purity were determined by Agilent LCMS constituting LC 1260 infinity and MS SQD 6120. ¹H NMR and ¹³C NMR were recorded on Bruker 500 (125 MHz) spectrometer in deuterated chloroform and deuterated methanol. Chemical shift values are expressed in terms of parts per million and J values are in Hertz. Splitting patterns are abbreviated as: s—singlet, d—doublet, dd—double doublet, t—triplet and m—multiplet.

### (Z)-2-{4-(4-chloro-1,2-diphenylbut-1-enyl) phenoxy}ethanol (1)

Compound 1 (Ospemifene) was prepared following a previously reported method (Eklund and Nilsson, PCT WO 2011/089385).

### (Z)-2-{4-(4-chloro-1, 2-diphenylbut-1-enyl}phenoxy) ethyl methanesulfonate (2)

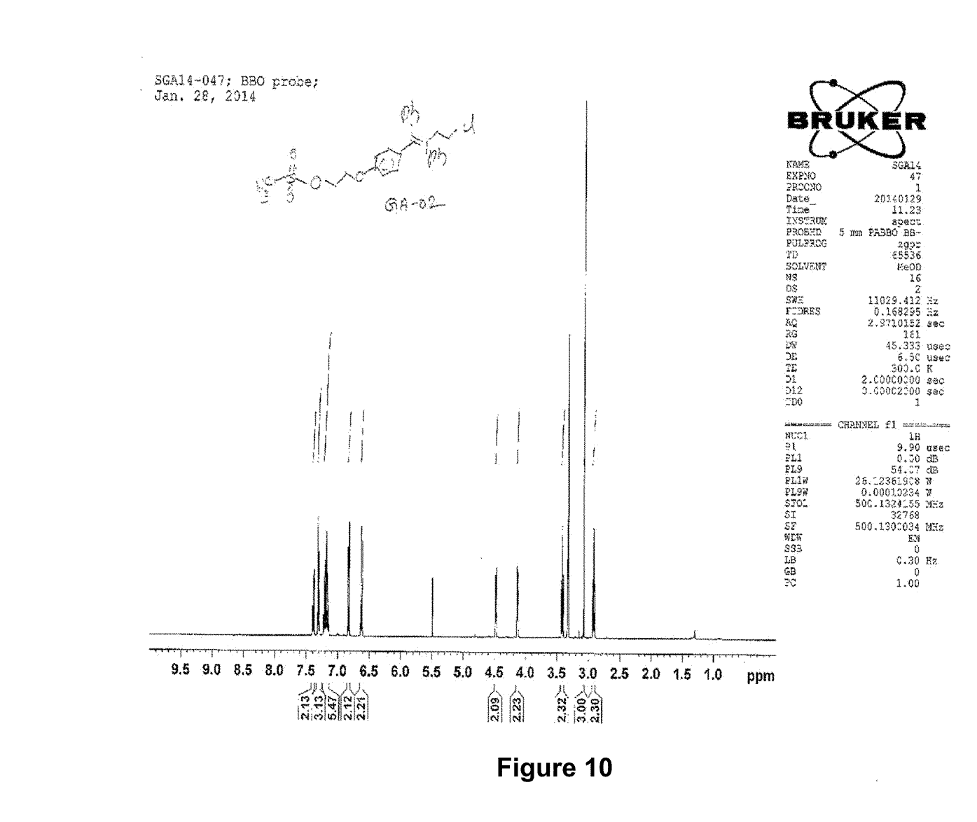

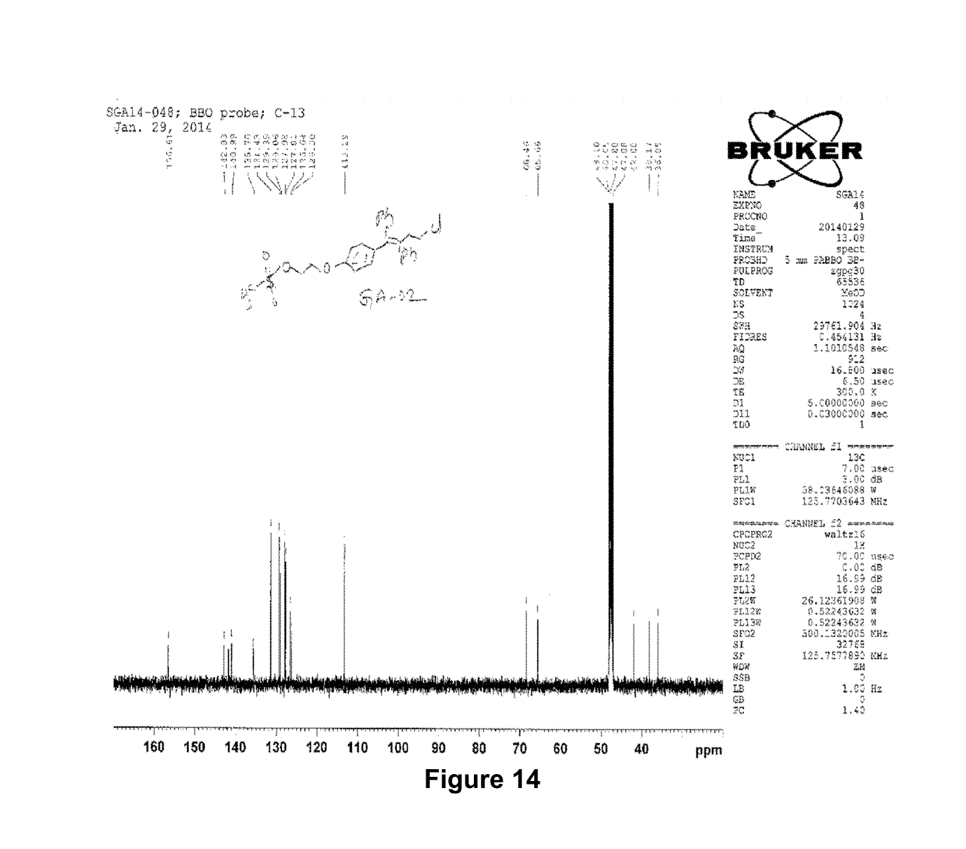

To a homogenous solution of 1 (1 mmol) in dry CH₂Cl₂ stirred at 0° C. under nitrogen atmosphere was added 2 mmol of trimethylamine followed by drop wise addition of 1.1 mmol of methane sulfonyl chloride dissolved in CH₂Cl₂. After the addition was complete, the reaction mixture was allowed to stir at room temperature for 4 h. Upon completion of reaction, as evidenced by TLC, the reaction mixture was cooled and added slowly over crushed ice. The solid precipitated was extracted with CH₂Cl₂ and the organic layer was dried over anhydrous sodium sulfate. The solvent was removed under reduced pressure resulting in a sticky solid mass which was recrystallized from hexane. White solid, Yield 80%; m.pt 84-86° C.; IR (KBr) vₘₐₓ: 3055, 1604, 1508 cm⁻¹; ¹H NMR (500 MHz, MeOD): δ_{H} 2.92 (t, 2H, ═CH—CH₂—CH₂—), 3.07 (s, 3H, H₃C—SO₂—), 3.42 (t, 2H, —CH₂—CH₂—H₂C—Cl), 4.14 (m, 2H, —H₂C—O-Ph), 4.48 (m, 2H, —H₂CS(O)₂—CH₃), 6.63 (d, 2H, J=7.5 Hz, c″), 6.83 (d, 2H, J=7.5 Hz, b″), 7.19 (m, 5H, b′, c, d′), 7.31 (m, 3H, J=8.0, 1.0 Hz, b, d), 7.39 (dd, 2H, J=7.5, 7.5 Hz, c′); ¹³C NMR (125 MHz, CD₃OH): δ_{C} 36.0, 38.2, 42.0, 65.7, 68.5, 113.3, 126.3, 126.6, 127.8, 128.0, 129.1, 129.4, 131.4, 135.6, 135.7, 141.0, 142.8, 156.6. MS m/z: 457 (M⁺). Analysis calculated for C₂₅H₂₅ClO₄S: C, 65.71; H, 5.51; S, 7.02. Found: C, 65.69; H, 5.48; S, 7.05. See FIG. 10 for ¹H NMR spectrum and FIG. 14 for ¹³C NMR spectrum.

### (Z)-[1-{4-(2-azidoethoxy) phenyl}-4-chlorobut-1-ene-1, 2-diyl]dibenzene (3)

To a stirred solution of 2 (1 mmol) in dry DMF was added 2 mmol of sodium azide and the mixture was allowed to stir at 60° C. for 4 h. Upon completion of the reaction, monitored by TLC, the reaction mixture was poured over ice and extracted with ethyl acetate. The extract was washed with brine and water, and dried over anhydrous sodium sulfate. The solvent was removed under reduced pressure and the oily mass was recrystallized from a mixture (1:9) of CH₂Cl₂ and hexane to obtain solid product 3. White solid, Yield 85%; m.pt 88-90° C.; IR (KBr) vₘₐₓ: 2921, 2111, 1604, 1506 cm⁻¹; 1^{H} NMR (500 MHz, CDCl₃): δ_{H} 2.95 (t, 2H, J=7.5 Hz, ═CHCH₂—CH₂—), 3.45 (t, 2H, J=7.5 Hz, CH₂—CH₂—H₂C—Cl), 3.53 (t, 2H, J=5.0 Hz, —H₂C—N₃), 4.03 (t, 2H, J=5.0 Hz, —H₂C—O-Ph), 6.59 (d, 2H, c″, J=7.5 Hz), 6.83 (d, 2H, b″, J=7.5 Hz), 7.16 (dd, 2H, J=8.0, 2.0 Hz, b′), 7.18 (dd, 1H, J=7.0, 7.0 Hz, d′), 7.23 (m, 2H, J=8.0, 3.0, 1.5 Hz, c), 7.32 (m, 3H, J=8.0, 2.0 Hz, b, d), 7.40 (dd, 2H, J=7.5, 7.5 Hz, c′); ¹³C NMR (125 MHz, CDCl₃): δ_{C} 35.3, 49.9, 61.4, 68.9, 113.5, 126.6, 127.0, 128.3, 129.3, 129.5, 131.8, 135.3, 141.2, 141.6, 142.9, 156.8. MS m/z: 404 (M⁺). Analysis calculated for C₂₄H₂₂ClN₃O: C, 71.37; H, 5.49; N, 10.40. Found: C, 71.32; H, 5.40; N, 10.30.

### (Z)-2-{4-(4-azido-1,2-diphenylbut-1-enyl) phenoxy}ethanol (4)

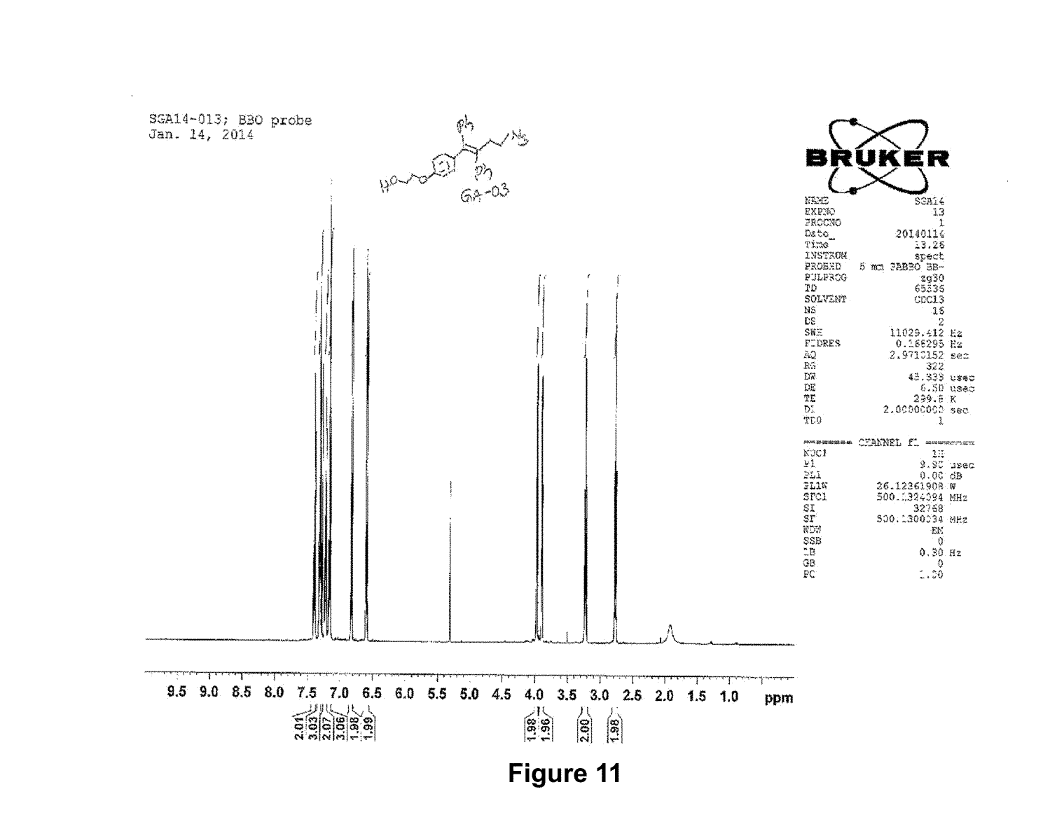

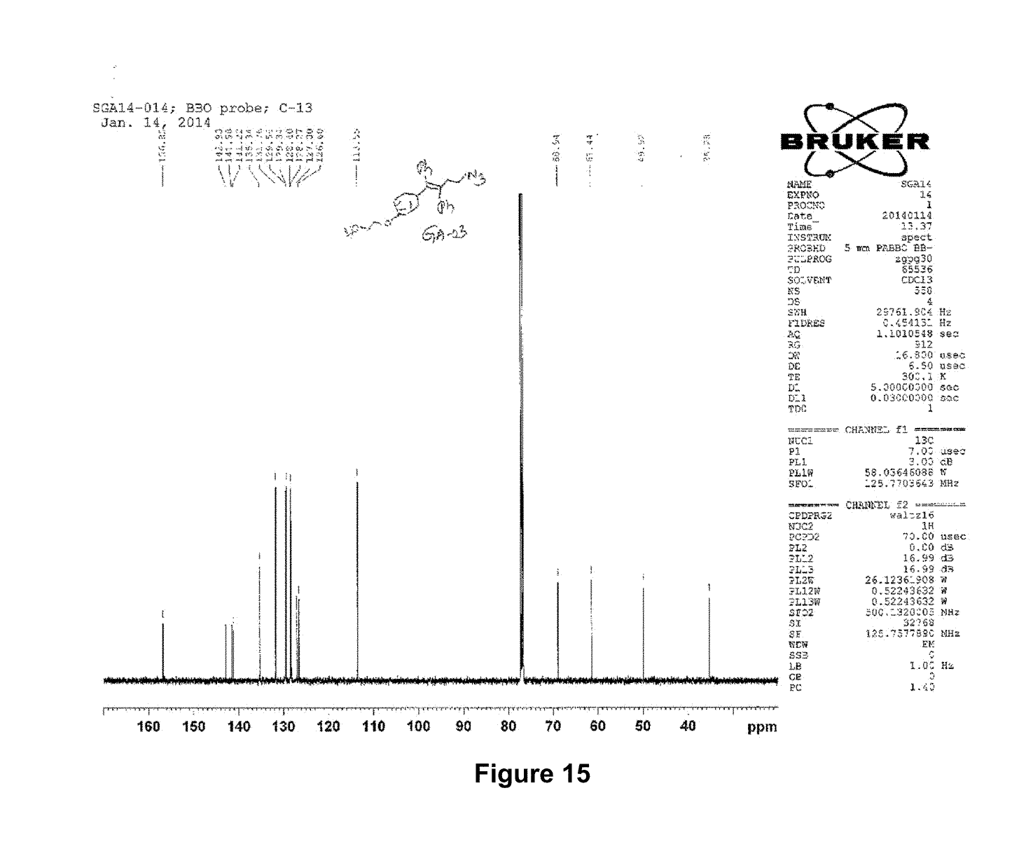

To a solution of 1 (1 mmol) in dry DMF, was added 4 mmol of sodium azide and the mixture was allowed to stir at 80° C. for 12 h. The progress of the reaction was monitored by TLC and on completion, the reaction mass was poured over ice and extracted with ethyl acetate. The extract was washed with brine and water and dried over anhydrous sodium sulphate. The removal of solvent under reduced pressure resulted in an oily mass which was recrystallized from a mixture (1:9) of CH₂Cl₂ and hexane. White solid, Yield 60%; m.pt 126-128° C.; IR (KBr) vₘₐₓ: 2102, 1604, 1574, 1506 cm⁻¹; ¹H NMR (500 MHz, CDCl₃): δ_{H} 1.70 (s, 1H, —OH), 2.77 (t, 2H, J=7.0 Hz, ═CH—CH₂—CH₂—), 3.23 (t, 2H, J=7.0 Hz, H₂C—N₃), 3.89 (t, 2H, J=5.0 Hz, —H₂C—OH), 3.97 (t, 2H, J=5.0 Hz, —H₂C—O-Ph), 6.60 (d, 2H, J=8.5 Hz, c″), 6.83 (d, 2H, J=8.5 Hz, b″), 7.16 (dd, 2H, J=8.0, 1.5 Hz, b′), 7.18 (dd, 1H, J=7.0, 7.5 Hz, d′), 7.23 (dd, 2H, J=7.5, 2.0 Hz, c), 7.31 (m, 3H, J=7.5, 7.5, 1.5 Hz, b, d), 7.40 (dd, 2H, J=7.5, 7.5 Hz, c′); ¹³C NMR (125 MHz, CDCl₃): δ_{C} 38.6, 42.8, 50.2, 66.7, 113.6, 126.6, 127.0, 128.2, 128.4, 129.4, 129.5, 131.7, 135.4, 140.9, 141.7, 142.9, 156.5. MS m/z: 386 (M⁺). Analysis calculated for C₂₄H₂₃N₃O₂: C, 74.78; H, 6.01; N, 10.90. Found: C, 74.75; H, 6.04; N, 10.85. See FIG. 11 for ¹H NMR spectrum and FIG. 15 for ¹³C NMR spectrum.

### (Z)-[4-azido-1-{4-(2-azidoethoxy) phenyl}but-1-ene-1,2-diyl]dibenzene (5)

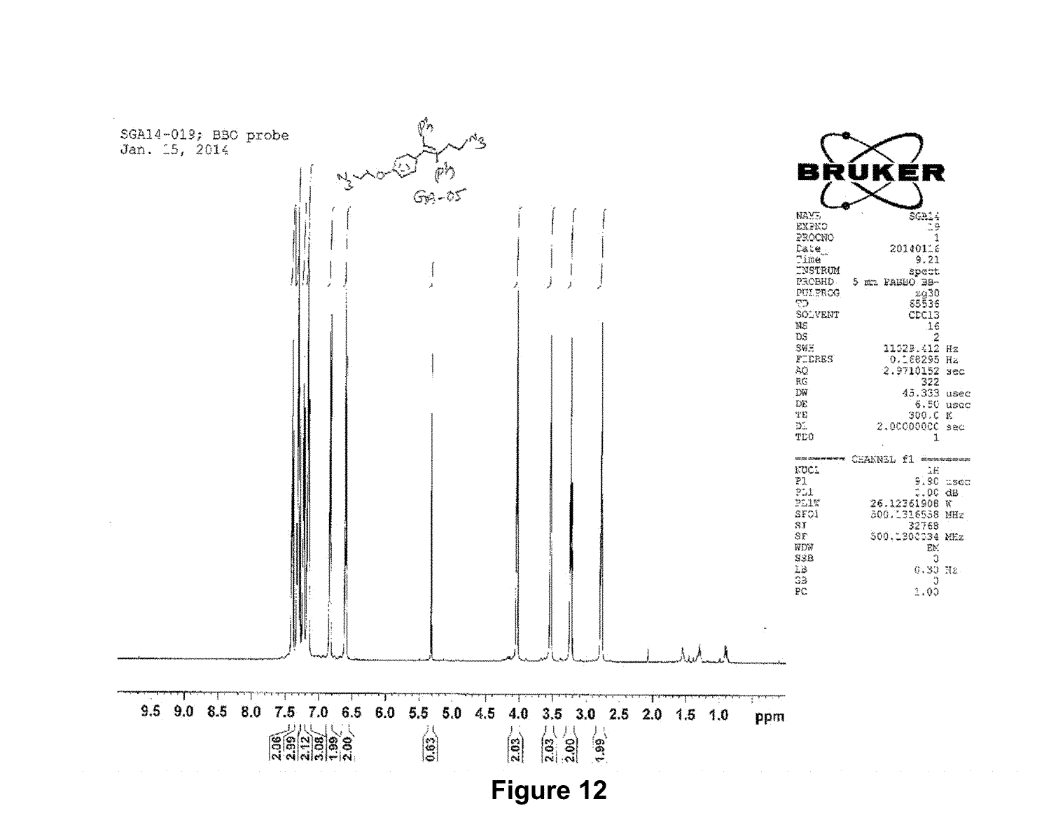

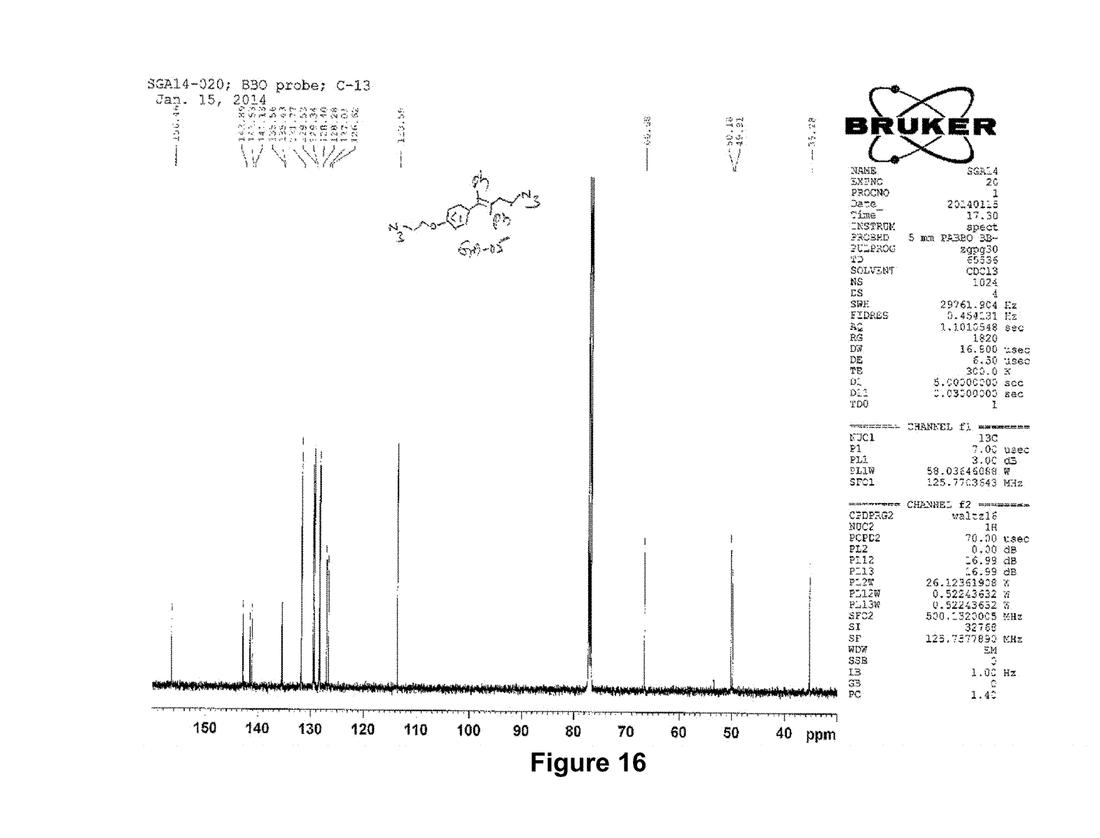

To a stirred solution of 3 (1 mmol) in anhydrous DMF, was added 5 mmol of sodium azide and the mixture was heated at 80° C. with constant stirring for 12 h. After the completion of reaction, as evidenced by TLC, the reaction mixture was quenched by pouring into ice cold water and extracted with ethyl acetate. The organic extract was washed with brine solution and water. It was then dried over anhydrous sodium sulphate and concentrated under reduced pressure to yield crude yellow oily mass which was recrystallized using a mixture (1:9) of CH₂Cl₂ and hexane. White solid, Yield 75%; m.pt 99-101° C.; IR (KBr) vₘₐₓ: 2099, 1604, 1506 cm⁻¹; 1H NMR (500 MHz, CDCl₃): δ_{H} 2.77 (t, 2H, J=7.5 Hz, ═CH—CH₂—CH₂—), 3.23 (t, 2H, J=7.5 Hz, H₂C—H₂C—N₃), 3.53 (t, 2H, J=5.0 Hz, —O—H₂C—H₂C—N₃), 4.03 (t, 2H, J=5.0 Hz, —H₂C—O-Ph), 6.60 (d, 2H, J=8.5 Hz, c″), 6.83 (d, 2H, J=8.5 Hz, b″), 7.16 (m, 2H, J=8.0, 1.5 Hz, b′), 7.17 (dd, 1H, J=8.5, 7.0 Hz, d′), 7.27 (dd, 2H, J=7.5, 7.5 Hz, c), 7.29 (m, 3H, J=8.0, 1.5 Hz, b, d), 7.39 (dd, 2H, J=7.5, 7.5 Hz, c′); ¹³C NMR (125 MHz, CDCl₃): δ_{C} 35.3, 49.9, 50.2, 66.7, 113.6, 126.6, 127.0, 128.3, 128.4, 129.3, 129.5, 131.8, 135.4, 135.6, 141.2, 141.5, 142.9, 156.5. MS m/z: 411 (M⁺). Analysis calculated for C₂₄H₂₂N₆O: C, 70.23; H, 5.40; N, 20.47. Found: C, 70.35; H, 5.26; N, 20.38. See FIG. 12 for ¹H NMR spectrum and FIG. 16 for ¹³C NMR spectrum.

General Procedure for the Synthesis of Compounds 6 and 7 (from 4 and 5, Respectively)

To a well stirred solution of 4 (1 mmol) (for 6) and 5 (1 mmol) (for 7) in aqueous ethanol (4:1 ethanol:water) were added 6 mmol of zinc dust and 7 mmol of ammonium chloride. The resulting suspension was refluxed for 8 h. Upon completion of reaction, monitored by TLC, ammonium hydroxide was added dropwise under vigorous stirring until the reaction mixture turned slightly alkaline in nature. The zinc dust was filtered and the filtrate was extracted with ethyl acetate. The aqueous layer was extracted twice with ethyl acetate and the combined organic extract was washed with brine followed by water, and dried over anhydrous sodium sulphate. The solvent was removed under vacuo and the solid product so obtained was recrystallized from a mixture (1:9) of CH₂Cl₂ and hexane.

### (Z)-2-{4-(4-amino-1, 2-diphenylbut-1-enyl) phenoxy}ethanol (6)

White solid, Yield 75%; m.pt 128-130° C.; IR (KBr) vₘₐₓ: 3499, 2925, 1605, 1508 cm⁻¹; 1^{H} NMR (500 MHz, MeOD): δ_{H} 1.89 (s, 2H, —H₂C—NH₂), 2.82 (m, 4H, H₂C═CH—CH₂—CH₂), 3.80 (t, 2H, J=5.0 Hz, —O—H₂C—H₂C—OH—), 3.92 (t, 2H, J=5.0 Hz, —H₂C—O-Ph), 6.63 (dd, 2H, J=7.0, 2.0 Hz, c″), 6.81 (dd, 2H, J=7.0, 2.0 Hz, b″), 7.23 (m, 7H, b, b′, c, d′), 7.34 (dd, 1H, J=7.5, 7.5 Hz, d), 7.43 (dd, 2H, J=7.5, 7.5 Hz, c′); ¹³C NMR (125 MHz, MeOD): δ_{C} 34.4, 38.6, 60.2, 69.0, 113.3, 126.5, 126.8, 128.0, 128.3, 128.7, 129.3, 131.3, 134.2, 134.6, 141.1, 142.3, 142.9, 157.5. MS m/z: 360 (M⁺). Analysis calculated for C₂₄H₂₅NO₂: C, 80.19; H, 7.01; N, 3.90. Found: C, 80.11; H, 7.06; N, 3.83

### (Z)-4-{4-(2-aminoethoxy) phenyl}-3, 4-diphenylbut-3-en-1-amine (7)

White solid, Yield 72%; m.pt 154-156° C.; IR (KBr) vₘₐₓ: 3499, 2925, 1605, 1508 cm⁻¹; ¹H NMR (500 MHz, MeOD): δ_{H} 1.89 (s, 4H, —H₂C—NH₂), 2.84 (m, 4H, H₂C═CH—CH₂—CH₂), 3.23 (b, 2H, H₂C—H₂C—NH₂), 4.07 (t, 2H, J=5.0 Hz, —O—H₂C—H₂C—NH₂), 6.68 (d, 2H, J=8.5 Hz, c″), 6.86 (d, 2H, J=8.5 Hz, b″), 7.18 (m, 5H, J=7.5, 1.5 Hz, d′, b′, c), 7.20 (dd, 2H, J=8.5, 1.5 Hz, b), 7.27 (dd, 1H, J=7.0, 7.5 Hz, d), 7.35 (dd, 2H, J=7.5, 7.5 Hz, c′); ¹³C NMR (125 MHz, MeOD): δ_{C} 33.9, 38.4, 39.1, 64.6, 113.4, 126.6, 126.9, 128.0, 128.4, 128.7, 129.3, 131.4, 134.3, 135.4, 140.9, 142.2, 142.7, 156.7. MS m/z: 359 (M⁺). Analysis calculated for C₂₄H₂₆N₂O: C, 80.41; H, 7.31; N, 7.81. Found: C, 80.30; H, 7.20; N, 7.75.

### (Z)-Phenyl 4-{4-(2-hydroxyethoxy) phenyl}-3, 4-diphenylbut-3-enylcarbamate (8)

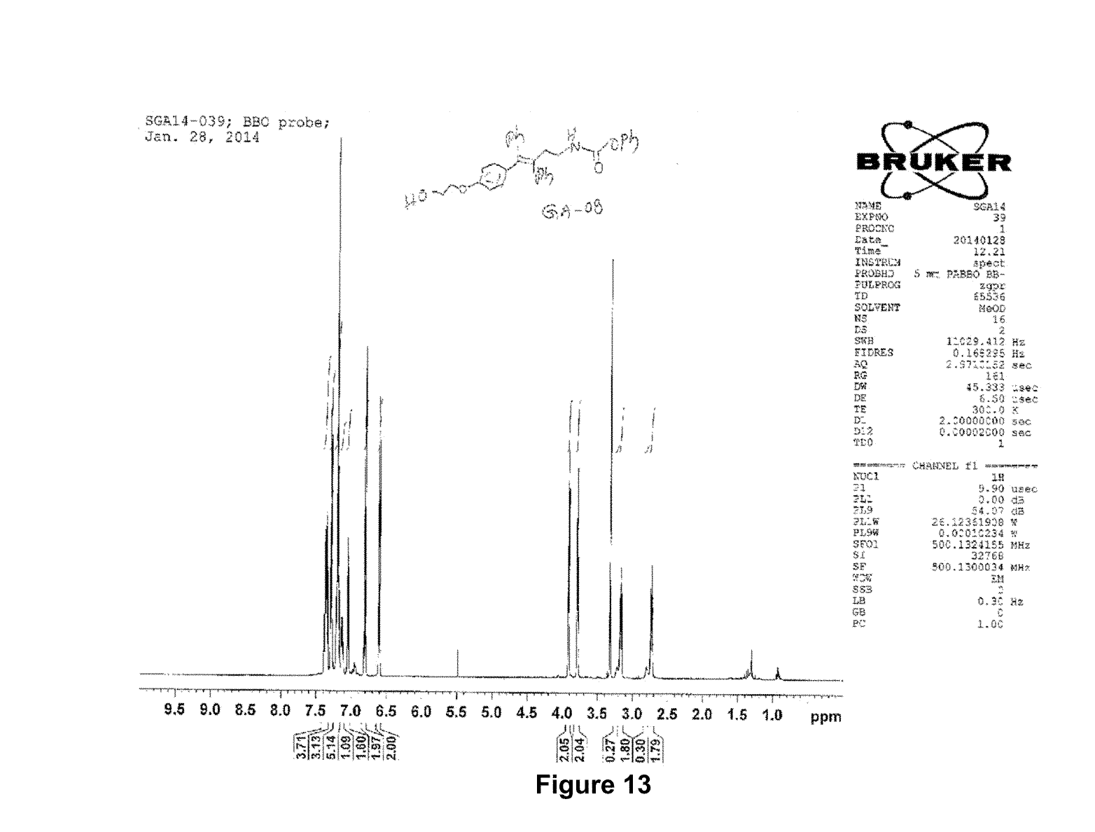

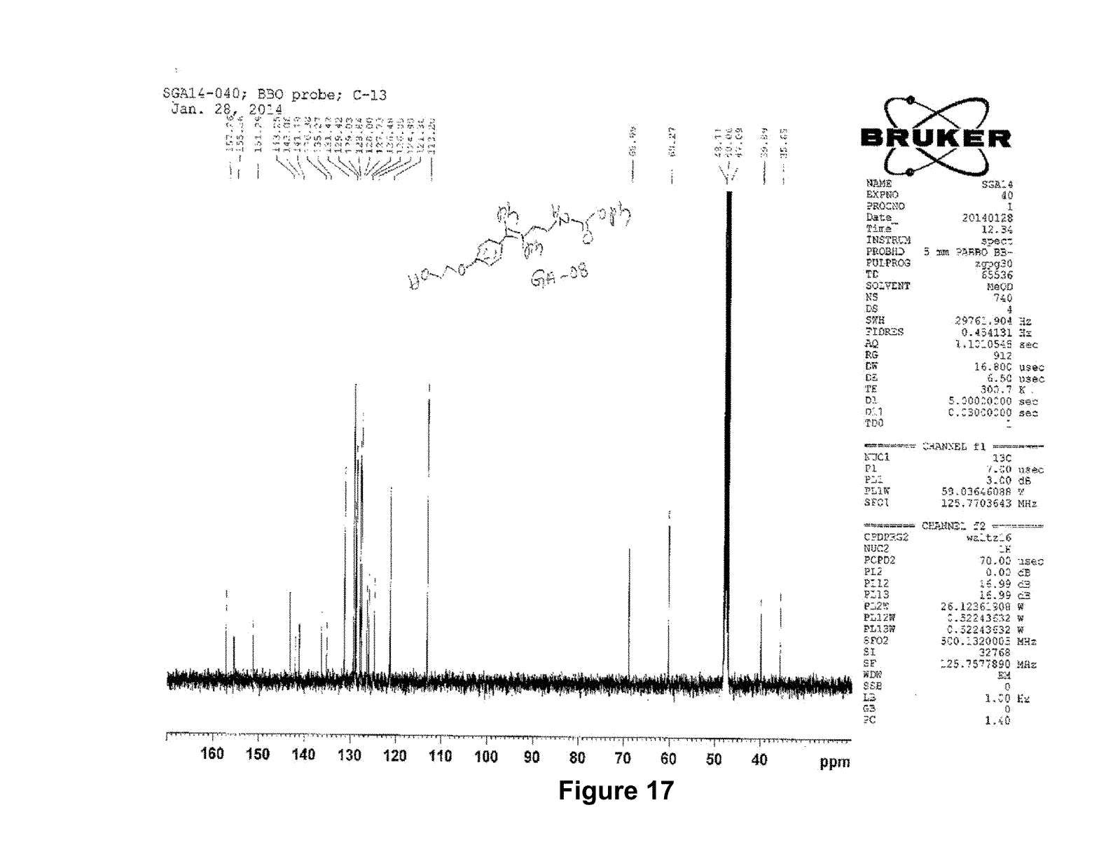

To a stirring suspension of compound 7, 3 mmol of K₂CO₃ in dry CHCl₃ at 0° C., was added 1.01 mmol of phenyl chloroformate. The reaction mixture was allowed to warm to room temperature and stirred for 2 h. Upon completion of reaction, as evidenced by TLC, K₂CO₃ was filtered off and the filtrate washed with water and dried over sodium sulphate. The solvent was removed under vacuo and the solid product 8 so obtained was recrystallized from a mixture (1:9) of CH₂Cl₂ and hexane. White solid, Yield 75%; m.pt 121-123° C.; IR (KBr) vₘₐₓ: 3307, 1677, 1604, 1506 cm⁻¹; ¹H NMR (500 MHz, CDCl₃): δ_{H} 2.73 (t, 2H, J=7.5 Hz, ═CH—CH₂—CH₂—), 3.17 (t, 2H, J=7.5 Hz, H₂C—H₂C—NH—), 3.79 (t, 2H, J=4.5 Hz, —O—H₂CH₂C—OH), 3.92 (t, 2H, J=4.5 Hz, —H₂C—O-Ph), 6.61 (d, 2H, J=7.5 Hz, c″), 6.81 (d, 2H, J=7.5 Hz, b″), 7.05 (d, 2H, J=8.0 Hz, b⁰), 7.13 (m, 1H, d⁰), 7.19 (m, 3H, c⁰, b′, d′), 7.28 (dd, 3H, J=7.5, 7.0 Hz, b, d) 7.36 (dd, 2H, J=8.0, 8.0 Hz, c), 7.37 (dd, 2H, J=8.0, 7.5 Hz, c′); ¹³C NMR (125 MHz, CDCl₃): δ_{C} 35.6, 39.9, 60.3, 69.0, 113.2, 121.4, 124.8, 126.0, 126.5, 127.7, 128.0, 128.8, 129.0, 129.4, 131.4, 135.3, 136.4, 141.2, 143.2, 151.3, 155.6, 157.3. MS m/z: 480 (M⁺). Analysis calculated for C₃₁H₂₉NO₄: C, 77.64; H, 6.10; N, 2.92. Found: C, 77.54; H, 6.00; N, 2.85. See FIG. 13 for ¹H NMR spectrum and FIG. 17 for ¹³C NMR spectrum.

### Cell Culture

MDA-MB-231 and MCF-7 cell lines maintained in DMEM medium supplemented with 10% fetal bovine serum (FBS) and 100 units/ml penicillin, 100 μg/ml streptomycin at 37° C. and 5% CO₂. These cells were obtained from ATCC. The cells were routinely screened for mycoplasma using Hoechst 33258 staining.

### Cell Viability Assay

The effects of Ospemifene analogs on the cell viability were determined by the MTT uptake method as previously described (Pandey et al., 2013, PloS One 8:e78570). Briefly, 3000 cells were incubated with various concentrations of Ospemifene analogs in triplicate in a 96-well plate for 48 h and 96 h at 37° C. An MTT solution was added to each well and incubated for 3 h at 37° C. After 3 h, DMSO was added and the optical density was measured at 570 nm using a 96-well multiscanner (Dynex Technologies, MRX Revelation; Chantilly, Va., USA). Backgrounds were subtracted at 630 nm. IC₅₀ values were calculated by non-linear regression analysis using Prism software.

### Live/Dead Assay

To measure cell death, the Live/Dead assay (Life technologies, USA) was used, which determines intracellular esterase activity and plasma membrane integrity (Pandey et al., 2013, PloS One 8:e78570). Nonfluorescent polyanionic dye calcein AM is retained by live cells and by enzymatic conversion (esterase) it becomes fluorescent, produces intense green fluorescence in live cells. Ethidium homodimer enters cells with damaged membranes and binds to nucleic acids, thereby producing a bright red fluorescence in dead cells. Briefly, 2×10⁵ cells were incubated with compounds 6 and 7, and control Ospemifene for 24 h at 37° C. Cells were stained with the Live and Dead reagent (5 μM ethidium homodimer and 5 μM calcein-AM) and analyzed by flow cytometry according to the manufacture's protocols.

### Docking Method

The crystal structures of ERα (pdb id: 3K6P) and ERβ (pdb id: 1 UOM) were retrieved from the protein data bank (http://www.rcsb.org). The native ligands and water molecules were removed from the proteins. Both proteins were protonated at physiological pH using the Prepare Protein algorithm in DS. The minimization of both proteins was performed using the conjugate gradient algorithm to remove the bad contacts using the CHARMm force field. All synthesized compounds were geometrically optimized at DFT level using the combination of B3LYP functional and 6-31g [d, p] basis sets, in Gaussian 09 (Frisch et al., 2009, Gaussian 09, Revision D.01, Gaussian, Inc., Wallingford, Conn.). Prior to docking, a binding sphere covering all the active site residues was generated using the Define and Edit Binding Site module embedded in DS. Docking of all compounds was subsequently performed using the CDOCKER algorithm (CHARMm-based docking) (Wu et al., 2003, J. Comput. Chem. 24:1549-1562), in DS. The new conformations of compounds were generated using the molecular dynamics methods, and were refined using the simulated annealing method at 300 K. Of the 10 best poses, selected based on their scoring function (-CDOCKER energy), the best pose was used for the binding energy calculations and further analysis.

The results of the experiments are now described.

### Synthetic Chemistry

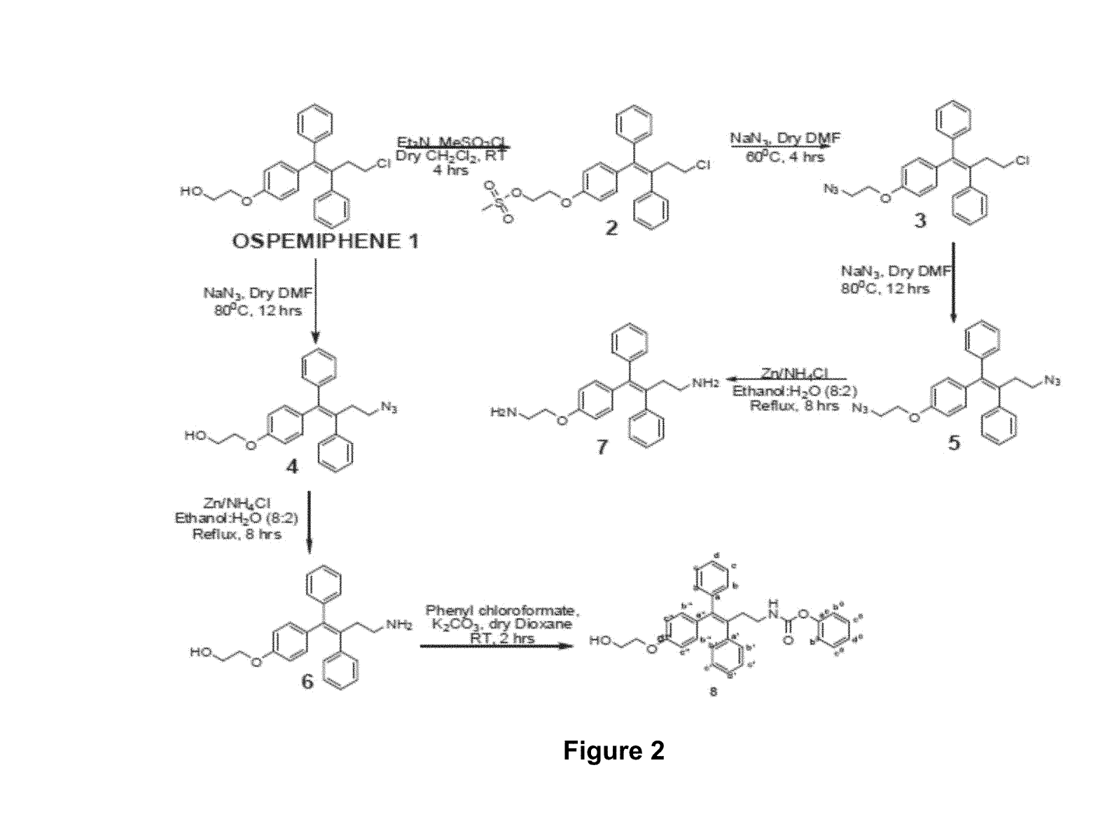

Ospemifene (1) obtained through a reported method (Eklund and Nilsson, PCT WO 2011/089385), by following the well documented McMurry reaction, served as the starting material for the synthesis of its desired novel analogs. Thus, the mesylate 2 was obtained by the treatment of 1 with an equivalent amount of methanesulphonyl chloride in dry CH₂Cl₂ at 0° C. in the presence of triethylamine. The treatment of mesylate 2 with sodium azide in dry DMF at 60° C. resulted in the azide 3. Similarly, the treatment of 1 with sodium azide led to the replacement of its chlorine group with azide to yield compound 4. In continuation of these studies and in pursuit of the desired goal, the diazide 5 was synthesized through monoazide 3 by replacement of its chlorine with azide. Further, the azides synthesized above were reduced to the corresponding amines 6 and 7 by the treatment of 3 and 5, respectively, with zinc dust and ammonium chloride in a mixture (4:1) of ethanol and water (FIG. 2). Amine 6 was treated with phenyl chloroformate to obtain amide 8.

### Pharmacology

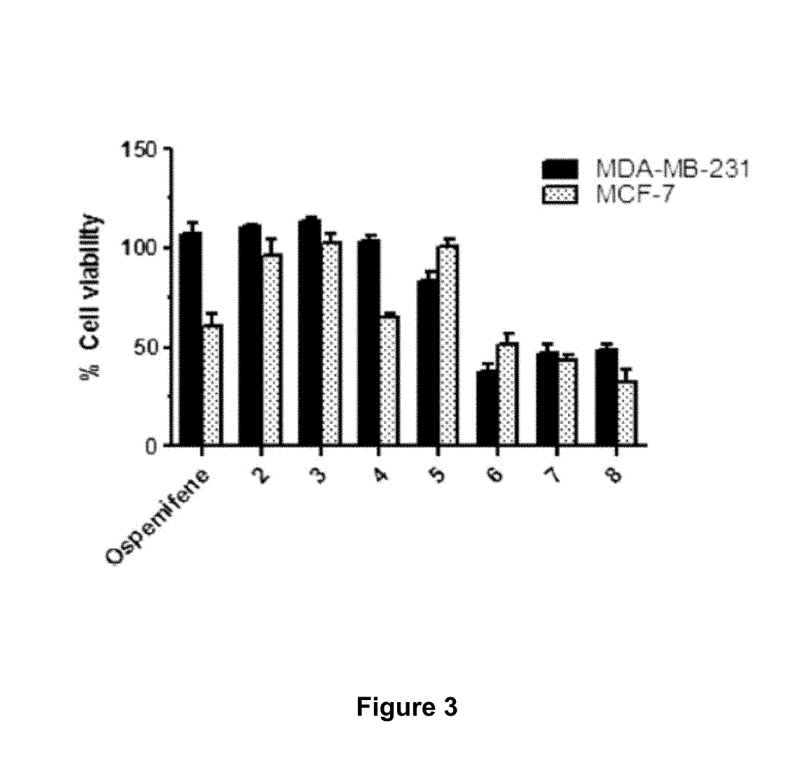

The synthesized compounds were evaluated for their anticancer activity on MCF-7 (ER+) and MDA-MB-231 (ER−) human breast cancer cell lines. Both ER+ and ER− cell lines were used to evaluate if the novel analogs were selectively cytotoxic to the ER+ cells similar to Ospemifene. For initial screening, cells were treated with eight concentrations of the compounds (0.5-25 μM) for 48 h. As previously reported, Ospemifene was more cytotoxic to ER+ MCF cells as compared to MDA-MB-231 cells showing ˜40% cell death at 25 μM concentration (FIG. 3). Although the compounds 2 and 3 were shown to be inactive against both the cell lines, however, the compound 4 obtained by the replacement of chloro by azide, showed activity similar to Ospemifene against MCF-7 cell lines. Although not wishing to be bound by any particular theory, these results suggest that the introduction of azide group replacing chloro group in Ospemifene was ineffective in enhancing the anticancer activity against the two studied cell lines. In addition, the replacement of hydroxyl group with azide group did not enhance the anticancer activity as compared to Ospemifene. However, the compound 5 was also shown to be ineffective against both the cell lines. Although not wishing to be bound by any particular theory, these results suggest that the replacement either or both of chloro and hydroxyl groups of Ospemifene with the azide group is not a promising strategy for the realization of the desired anti-breast cancer activity. The compounds 6, 7, and 8 were found to be more potent than Ospemifene. Interestingly, these compounds were effective in inhibiting cell viability of both the cell types, compound 8 being only slightly more selective to MCF-7 cells (FIG. 3).

Based on these results, compounds 4, 6, 7, and 8, having similar or better potency than Ospemifene in one or both the cell lines, were selected and subjected to an in-depth cell viability MTT assay with a wider range of concentration of up to 100 μM for two time points 48 and 96 h. Ospemifene and antagonist of ER, Tamoxifen, were used as positive controls. It is noteworthy that while Ospemifene is selectively cytotoxic to MCF-7 cells, replacing —Cl in Ospemifene by —NH2 group (as in 6) or both —Cl and —OH groups by —NH2 groups (as in 7) rendered the compounds cytotoxic to both the cell lines. Compounds 6 and 7 were found to be at least five times more cytotoxic than Tamoxifen to both the cell lines and about five to eight times more effective than Ospemifene in MCF-7 and MDA-MB231 cells, respectively, at the 96 h time point (Table 1).

TABLE 1
^{a}IC₅₀ of Ospemifene, Tamoxifen, and compounds 4-8 in breast cancer cells,
and their computed binding energies (BE) for both receptors (ERα and ERβ).

| | MDA-MB- | | | | | |
|---|---|---|---|---|---|---|
| | 231 (ER− | | MCF-7 (ER | | | |
| | negative) | | positive) | | | |
| | IC₅₀ (μM) | | IC₅₀ (μM) | | ERα | ERβ |
| Analogs | 48 h | 96 h | 48 h | 96 h | BE (kcal mol⁻¹) | BE (kcal mol⁻¹) |
| 4 | >100 | >100 | >100 | >100 | — | — |
| 6 | 25 | 14.5 | 15.9 | 12.4 | −117.1 | −97.1 |
| 7 | 17.1 | 13.4 | 23.6 | 11.2 | −132.2 | −99.2 |
| 8 | >100 | 62.3 | 76 | 50 | −99.5 | −81.2 |
| Ospemifene | >100 | >100 | >100 | 55 | −81.0 | −79.0 |
| Tamoxifen | 84.6 | 75 | 82.5 | 64.3 | −87.0 | −73.5 |

^{a}The breast cancer cells were treated with compounds for 48 and 96 h and the IC₅₀ was determined by non-linear regression.

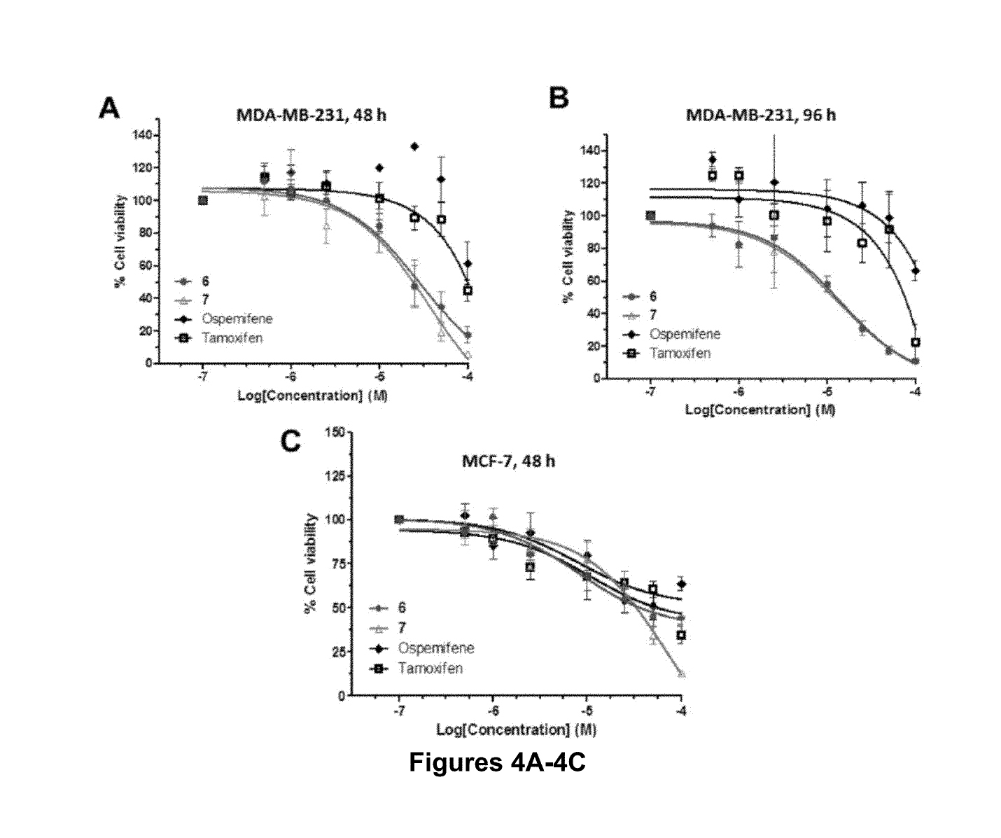

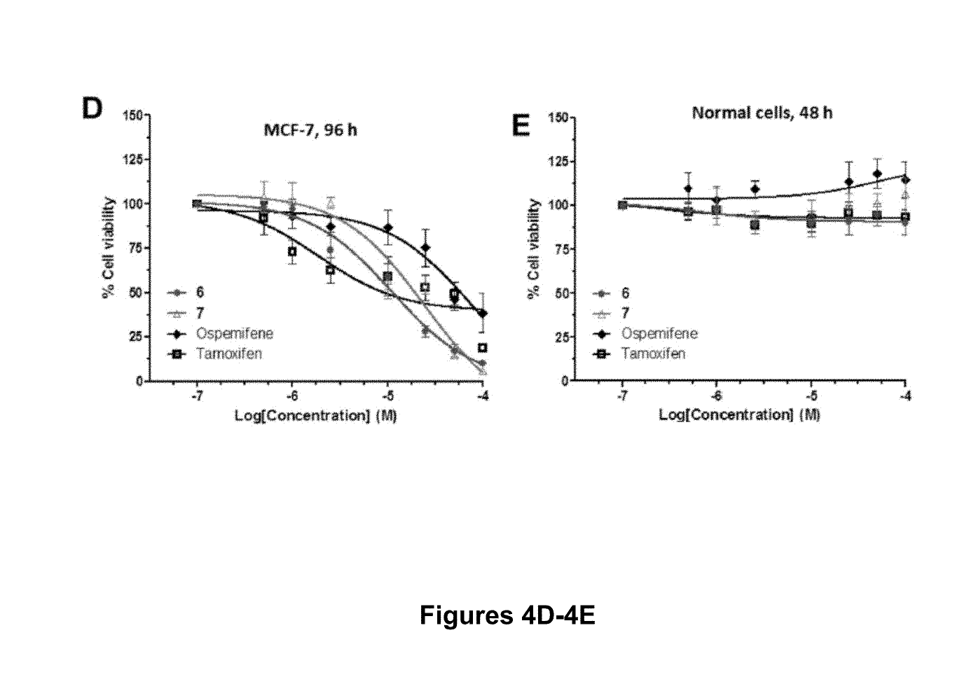

Compound 8 was only slightly more effective than Ospemifene and Tamoxifen and showed some affinity towards MCF-7 cells. Dose-response curves for data of compounds 6 and 7 in comparison to controls Ospemifene and Tamoxifen on both MCF-7 and MDA-MB231 cell lines are represented by a nonlinear regression plot in FIG. 4.

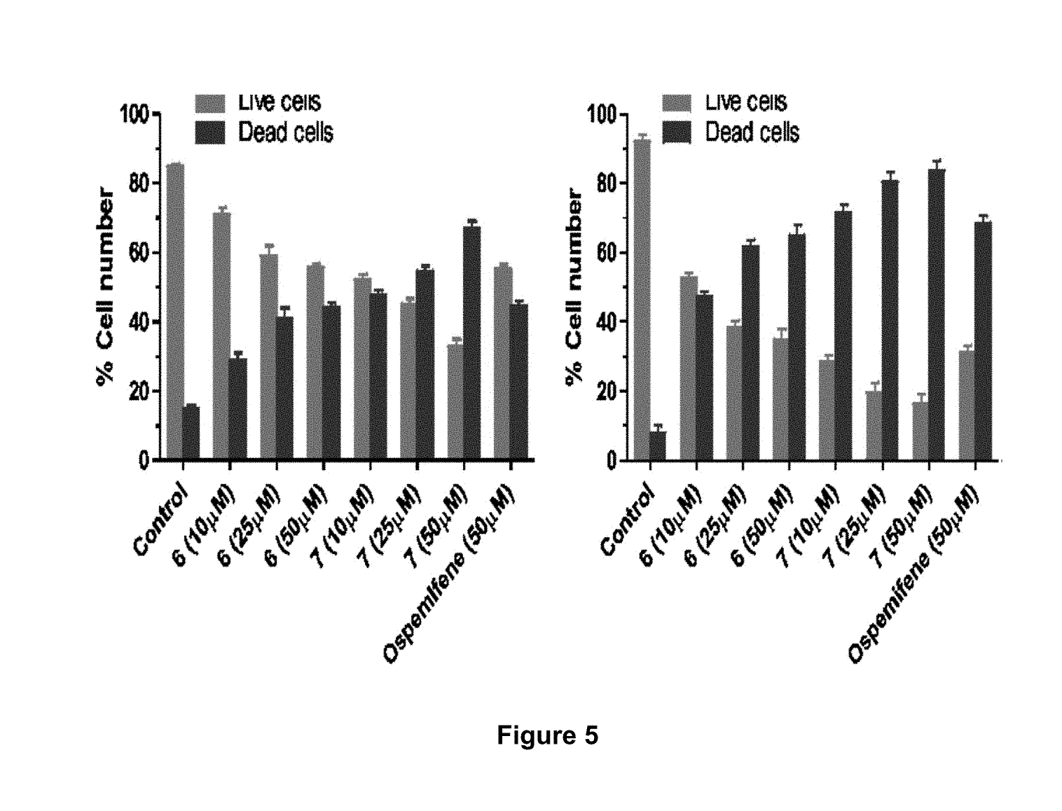

Based on the MTT assays, compound 7 followed by 6 emerged as the highly effective compounds. The selectivity of these compounds was further investigated in normal mouse embryonic fibroblast (MEF) cells. It was observed that compound 6, followed by 7, ospemifene, and tamoxifen were non-toxic to normal MEF cells (FIG. 3E). Although not wishing to be bound by any particular theory, this result suggests that the cytotoxic response of these compounds is selective for cancer cells. In addition, the cytotoxic response of compound 6 and 7 was tested by utilizing another method known as a live and dead assay. In this method, live cells are distinguished by the presence of ubiquitous intracellular esterase activity, determined by the enzymatic conversion of nonfluorescent cell-permeant calcein AM to the intensely fluorescent calcein. The polyanionic dye calcein is well retained within live cells only, producing an intense uniform green fluorescence in live cells (ex/em ˜495 nm/˜515 nm). Ethidium homodimer-1 (EthD-1) enters cells with damaged membranes producing a bright red fluorescence in dead cells (ex/em ˜495 nm/˜635 nm). EthD-1 is excluded by the intact plasma membrane of live cells. Thus live cells give a strong green signal and dead cells produce red signals. Live and dead cells are presented as a graph in FIG. 5. As revealed in FIG. 5, a dose dependent response of compounds 6 and 7 was observed. Compound 6 was found relatively more effective in MCF-7 cells compared to in MDA-MB-231. Almost 50-70% MCF-7 cells were dead when treated with increasing amount of compound 6. Compound 7 was more potent in both cell types compared to compound 6, with even an amount as low as 10 μM was enough to kill 70% of cells. MCF-7 cells were more sensitive for compound 7, as compared to MDA-MB231. Higher concentration (50 μM) of Ospemifene was used as a positive control. As indicated, the response of Ospemifene was cell selective and more in MCF-7 cells.

### Molecular Docking Analysis

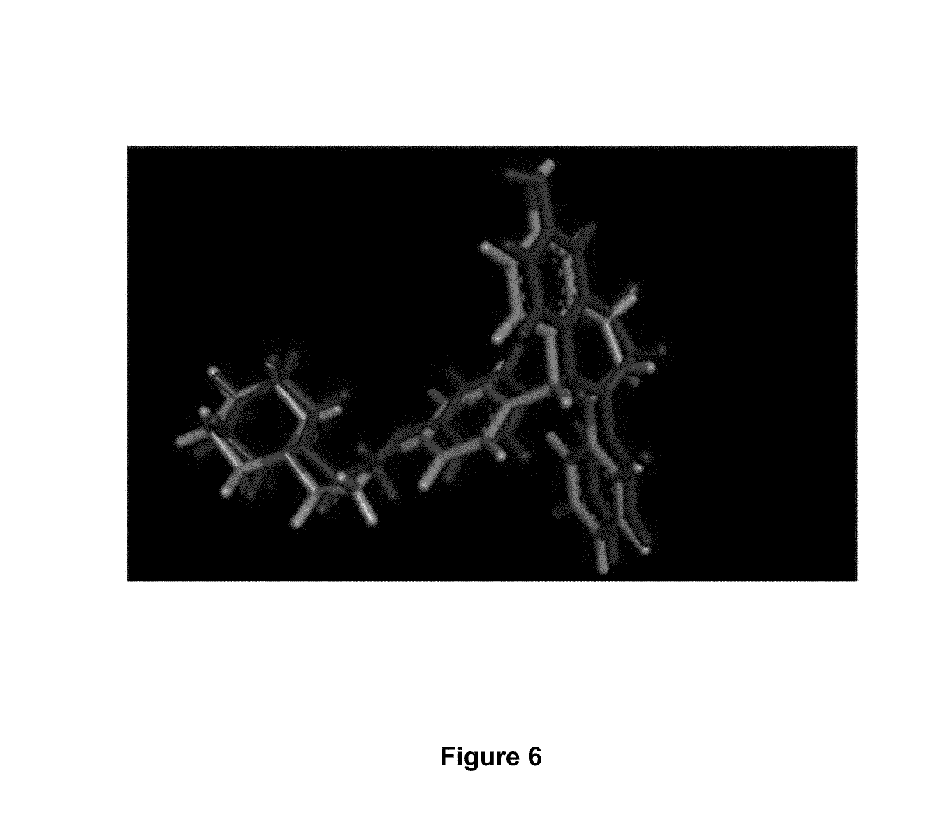

In order to substantiate the activity profiles of synthesized compounds, docking simulations were performed on the binding sites of both ERβ and ERα. Initially, the efficiency of docking protocol was assessed by re-docking the reference compound (tetrahydroisochiolin) in the active site of ERβ (pdb id: 1 UOM) using the CDOCKER module in Discovery Studio (DS) (Wu et al., 2003, J. Comput. Chem. 24:1549-1562). The computed root mean square deviation of the predicted binding conformation of the reference compound and its X-ray structure was around 0.65 Å (FIG. 6), and validated the docking procedure. All synthesized compounds were subsequently docked into the binding sites of ERβ and ERα (pdb id: 3K6P). The results obtained revealed that compounds bearing amine/amide moieties (6, 7 and 8) were more selective towards ERα than ERβ, whereas those bearing azide functionality (3, 4 and 5) did not show any affinity for either of the receptors. The computed binding energies for ERα range between −81.0 and −132.2 kcal mol⁻¹, whereas the binding energies predicted for ERβ were found to be between −45.4 and −99.2 kcal mol⁻¹ (Table 2).

TABLE 2
Docking results of Ospemifene, compounds 2-8 and Tamoxifen with both
ERα and ERβ receptors

| | ERβ | | | ERα | | |
|---|---|---|---|---|---|---|
| | | No of | | | No of | |
| | BE | Hydrogen | Interacting | BE | Hydrogen | Interacting |
| Compound | Kcal/mol | bonds | residues | Kcal/mol | bonds | residues |
| 1 | −79.0 | 2 | Asp351, Ala350, | −81.0 | 0 | Cys325, Val504, |
| (Ospemifene) | | | Leu525, Phe404, | | | |
| | | | Leu391, Phe425 | | | |
| 2 | −45.4 | 0 | Ala350, Leu525, | −77.5 | 0 | Cys325, Val504, |
| | | | Phe404, Phe425 | | | Phe495, Val321 |
| 3 | — | — | — | — | | — |
| 4 | — | — | — | — | | — |
| 5 | — | — | — | — | | — |
| 6 | −97.0 | 3 | Asp351, Ala350, | −117.1 | 0 | Cys325, Val504, |
| | | | Leu525, | | | Ala322, Val321 |
| | | | Phe404, His524, | | | |
| | | | Ile424 | | | |
| 7 | −99.2 | 2 | Asp351, Ala350, | −132.2 | 0 | Cys325, Val504, |
| | | | Leu525, Phe404, | | | Val321 |
| | | | Ile424 | | | |
| 8 | −81.2 | 1 | Asp351, Ala350, | −99.5 | 0 | Cys325, Leu398, |
| | | | Leu539, | | | Val321, Leu401, |
| | | | Leu525, Leu384 | | | Val491 |
| Tamoxifen | −73.5 | 4 | Asp351, Ala350, | −87.0 | 1 | Cys325, Val504, |
| | | | Leu525, Ile424, | | | Val321, |
| | | | Phe404, His524, | | | |
| | | | Leu387 | | | |

Although not wishing to be bound by any particular theory, these results suggest the favorable binding of compounds towards ERα. Compound 7 exhibited the strongest binding affinity with both receptors, followed by 6 and 8 (Table 1), in agreement with the cytotoxicity data.

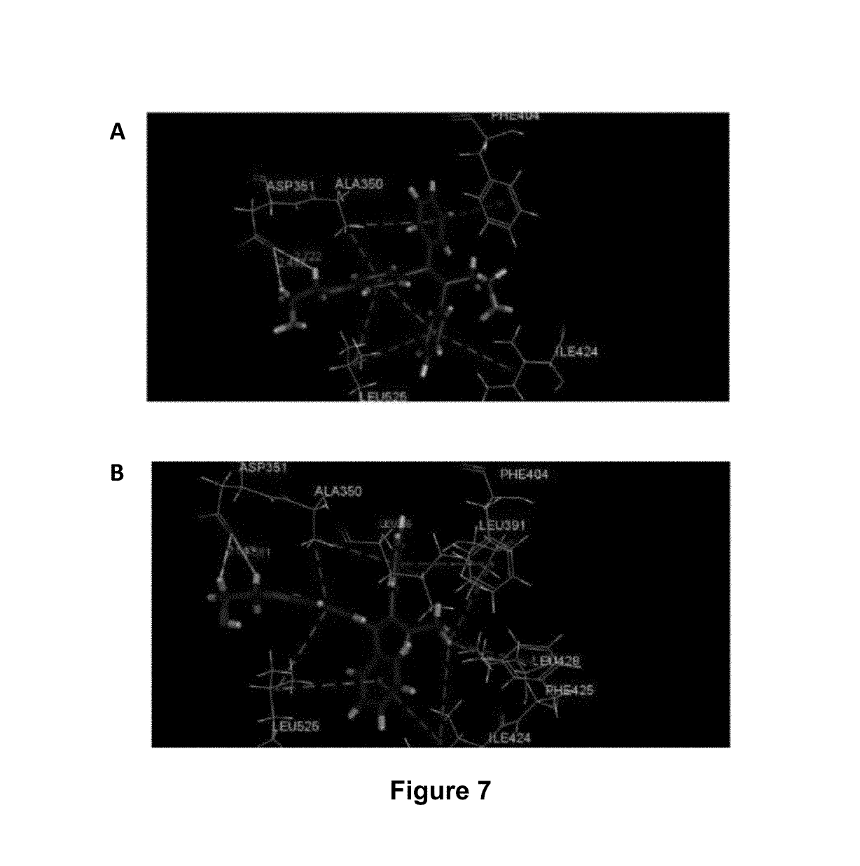

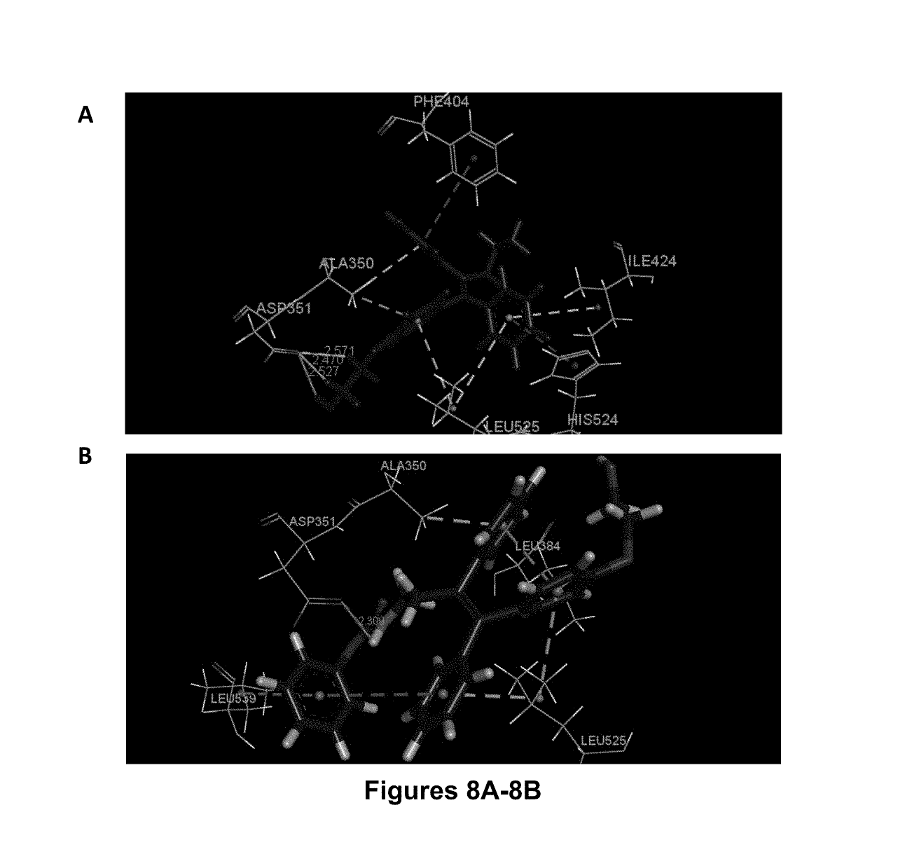

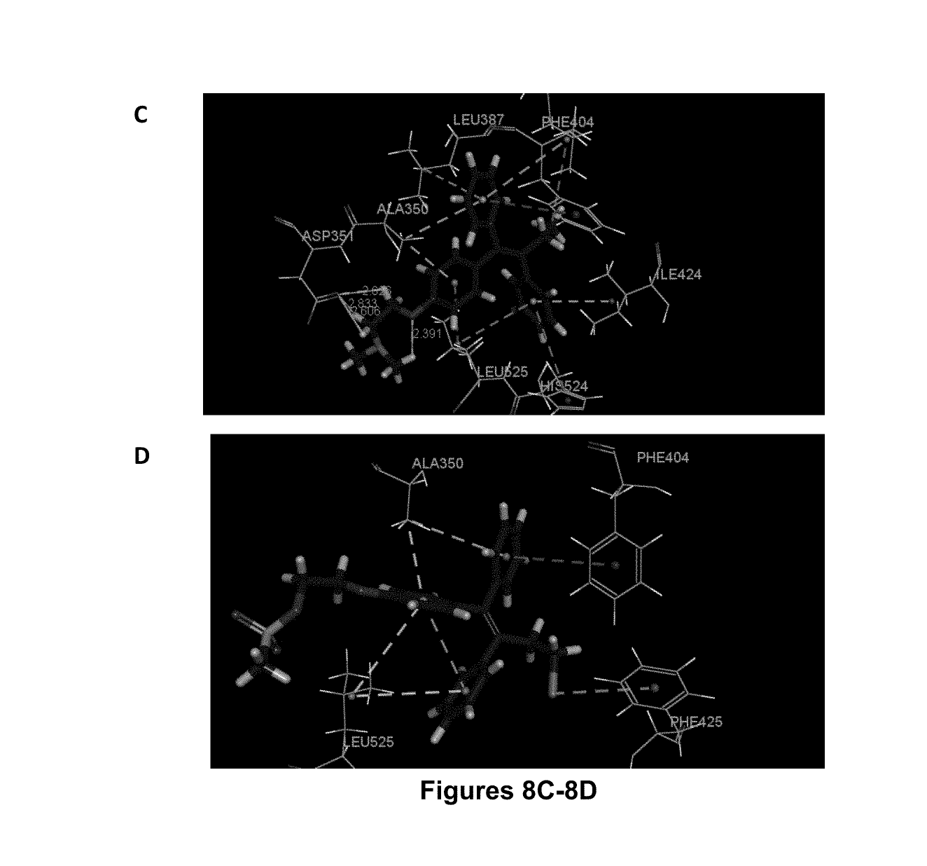

The docked complexes were further analyzed to get a deeper understanding of their host-guest relationship. All docked compounds, except 2, exhibited both hydrogen bonds and hydrophobic interactions with the ERβ. The predicted docking pose of compound 7 forms two hydrogen bonds with carbonyl oxygen of Asp351 and a potential π-π stacking through its phenyl ring with the aromatic side chain of Phe404 (FIG. 7A). Additionally, the phenyl rings of 7 displayed hydrophobic and van der Waals interactions with the Ile424, Leu525, Ala350 and Asp351 residues of ERβ. Ospemifene (FIG. 7B) and remaining compounds (6, 8, Tamoxifen; FIGS. 8A-8C), like 7, also interacted with similar amino acid residues of ERβ through hydrogen bonding and hydrophobic forces. It is hypothesized that the interaction of compounds with Asp351 is important for their stabilization in the binding site of ERβ (Desai et al., 2012, Int. J. Pharm. Sci. Rev. Res. 16:91-95), and could be related to their activity. The comparative relaxed conformation of 2 (FIG. 8D) misses its interaction with Asp351. Although not wishing to be bound by any particular theory, this result may account for its higher energy of binding (−45.4 kcal mol⁻¹) and its inactive nature under experimental conditions.

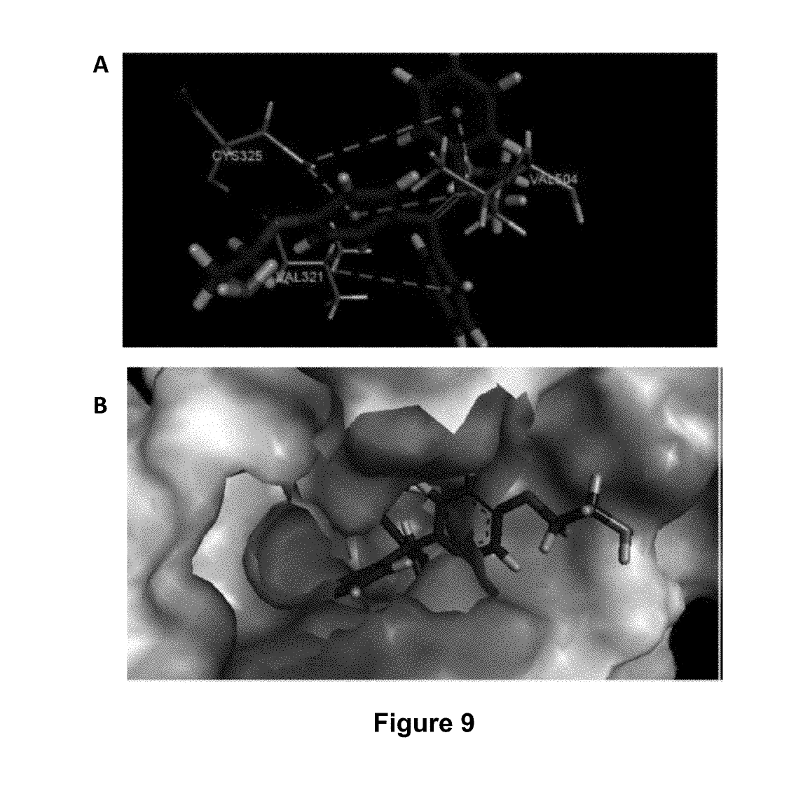

The docking results in case of ERα revealed the predominance of hydrophobic interactions. Compound 7 (FIG. 9A) was tightly inserted inside the binding cavity via hydrophobic interactions between its phenyl rings and side chains of Cys325, Val321 and Val504. The surface representation of ERα revealed the deep penetration of alkyl amine group of 7 (FIG. 9B) into the binding pocket which could have favored its efficient binding resulting in lower binding energy. The aromatic rings of Ospemifene and remaining compounds (2, 6, 8 and Tamoxifen) also exhibited hydrophobic interactions with similar residues. Although not wishing to be bound by any particular theory, the inability of azide compounds (3, 4 and 5) to dock with both target proteins supported their inactive behavior for breast cancer in the present study.

In conclusion, new structural analogues (2-8) of Ospemifene were prepared and screened for their activity against MCF-7 (ER-positive) and MDA-MB-231 (ER-negative) human breast cancer cell lines. Ospemifene was found to be toxic to MCF-7 cell lines but was not at all effective on MDA-MB-231 cell lines. Also, the compounds containing more polar groups like amine and amide (6, 7 and 8) were found to be more potent than Ospemifene against MCF-7 cells and were better even in case of non-estrogen dependent MDA-MB-231 cells. Although not wishing to be bound by any particular theory, the high potency observed in case of amines and amides could be due to their improved hydrogen bonding abilities. Finally, docking simulations performed on the ERα and ERβ revealed that compounds 6-8 were stronger inhibitors for both receptors, and supported the experimental findings.

### Example 2: Design, Synthesis and Evaluation of Triarylethylene Analogs as Anti-Breast Cancer Agents

The results described herein demonstrate the synthesis of novel triarylethylene analogs as potential anti-breast cancer agents. The cytotoxic potential of these analogs against ER-positive (MCF-7) and ER-negative (MDA-MB-231) human breast cancer cell lines was determined and compared with the well-known Selective Estrogen Receptor Modulators (SERMs), i.e. Ospemifene and Tamoxifen. In initial screening, analogs 5, 14 and 15 were found to be much more effective than the standards, Ospemifene and Tamoxifen against these cell lines. The results showed that these novel analogs inhibit the expression of proteins involved in the migration and metastasis, the compound 5 being most effective. The compound 5 inhibited the expression of MMP-9, c-Myc and Caveolin in both MCF-7 and MDA-MB-231 cells while compounds 14 and 15 only modulated the expression of MMP-9 in both cells. In addition, compound 5 suppressed the invasion of ER-negative cells in a dose dependent manner. The observed activity profiles were further supported by the docking studies performed against estrogen receptors (ERα and ERβ). The computed binding energies supported the experimental anti-cancer activity profiles of the compounds.

The materials and methods employed in these experiments are now described.

### Synthetic Chemistry

### General

Melting points were determined by open capillary using a Veego Programmable Melting/Boiling Point Apparatus and are uncorrected. IR spectra were recorded on Perkin Elmer FT-IR Spectrometer Spectrum Two. Molecular masses and purity were determined by Agilent LCMS constituting LC 1260 infinity and MS SQD 6120. ¹H NMR was recorded in deuterated chloroform (except 13 in deuterated methanol) on Bruker 500 MHz (except 11 and 13 on 600 MHz). ¹³C NMR spectra was recorded in deuterated chloroform (except 13 in deuterated methanol) on Bruker 125 MHz (except 11 and 13 on 150 MHz) spectrometer. Chemical shift values are expressed in terms of parts per million and J values are in Hertz. Splitting patterns are abbreviated as: s—singlet, d—doublet, dd—double doublet, t—triplet, q—quartet, b—broad and m—multiplet.

### (Z)-(4-Chloro-1-(4-methoxyphenyl)but-1-ene-1,2-diyl)dibenzene (11)

To a stirred suspension of zinc dust (6.5 mmol) in dry THF at −10° C., under dry nitrogen atmosphere, was added dropwise TiCl₄ (3.5 mmol) in about 20 minutes maintaining the temperature below 0° C. After the addition was complete, the reaction mixture was allowed to reflux for 2 h. The reaction temperature was lowered to 0° C. and a solution of reactants 9 (1 mmol) and 10 (1 mmol) in dry THF were added dropwise over a period of 20 min. On completion, the reaction mixture was again refluxed for 2 h. The progress of the reaction was monitored by TLC. The reaction mixture was then cooled to room temperature, transferred to stirred solution of 10% K₂CO₃ and filtered. The filtrate/solvent were concentrated under reduced pressure to recover THF. The crude material so obtained was dissolved in ethyl acetate and washed with brine followed by water, and dried over anhydrous sodium sulphate. The solvent was removed under reduced pressure and the obtained oily mass was recrystallized from a mixture (1:9) of water and methanol to obtain solid product 11. White solid, Yield 65%; m.pt 87-89° C.; IR (KBr) vₘₐₓ: 1604, 1508, 1490 cm⁻¹; ¹H NMR (600 MHz, CDCl₃): δ_{H} 2.96 (t, 2H, J=7.8 Hz, —H₂C—CH₂—), 3.46 (t, 2H, J=7.8 Hz, —H₂C—Cl), 3.71 (s, 3H, H₃C—O—), 6.59 (d, 2H, J=7.2 Hz, c″), 6.83 (d, 2H, J=7.2 Hz, b″), 7.24 (m, 8H, aromatic), 7.40 (dd, 2H, J=7.2 Hz, c); ¹³C NMR (150 MHz, CDCl₃): δ_{C} 38.6, 42.9, 55.0, 66.7, 112.9, 126.6, 126.9, 127.4, 128.2, 128.3, 128.4, 129.6, 130.5, 130.6, 131.7, 134.8, 135.2, 141.0, 141.8, 142.9, 157.8. MS m/z: 349 (M⁺). Analysis calculated for C₂₃H₂₁ClO: C, 79.18; H, 6.07. Found: C, 79.21; H, 6.05.

### (Z)-(4-Azido-1-(4-methoxyphenyl)but-1-ene-1,2-diyl)dibenzene (12)

To a solution of 11 (1 mmol) in dry DMF, was added sodium azide (5 mmol) and the mixture was allowed to stir at 80° C. for 12 h. The progress of the reaction was monitored by TLC and on completion, the reaction mass was poured over ice and extracted with ethyl acetate. The extract was washed with brine and water and dried over anhydrous sodium sulphate. The removal of solvent under reduced pressure resulted in an oily mass which was recrystallized from a mixture (1:9) of CH₂Cl₂ and hexane. White solid, Yield 80%; m.pt 126-128° C.; IR (KBr) vₘₐₓ: 2102, 1604, 1574, 1506 cm⁻¹; ¹H NMR (500 MHz, CDCl₃): δ_{H} 2.77 (t, 2H, J=7.0 Hz, —H₂C—CH₂—), 3.23 (t, 2H, J=7.0 Hz, —H₂C—N₃), 3.71 (s, 3H, H₃C—O—), 6.58 (d, 2H, J=7.8 Hz, c″), 6.83 (d, 2H, J=7.0 Hz, b″), 7.17 (m, 3H, b′, d′), 7.23 (dd, 2H, J=7.0, c′), 7.32 (m, 3H, b, d), 7.40 (dd, 2H, J=7.0 Hz, c); ¹³C NMR (125 MHz, CDCl₃): δ_{C} 35.3, 49.9, 55.0, 112.9, 126.6, 127.0, 128.3, 128.4, 129.4, 129.6, 131.7, 134.8, 135.1, 141.3, 141.7, 143.0, 157.8. MS m/z: 386 (M⁺). Analysis calculated for C₂₃H₂₁N₃O: C, 77.72; H, 5.96; N, 11.82. Found: C, 77.69; H, 5.92; N, 11.80.

### (Z)-4-(4-Methoxyphenyl)-3,4-diphenylbut-3-en-1-amine (13)

To a stirred solution of 12 (1 mmol) in aqueous ethanol (4:1: ethanol: water) were added zinc dust (6 mmol) and ammonium chloride (7 mmol). The resulting suspension was refluxed for 8 h. Upon completion of reaction, monitored by TLC, ammonium hydroxide was added dropwise under vigorous stirring until it turned slightly alkaline in nature. The zinc dust was filtered and the filtrate was extracted with ethyl acetate. The aqueous layer was extracted twice with ethyl acetate and the combined organic extract was washed with brine followed by water, and dried over anhydrous sodium sulphate. The solvent was removed under vacuo and the solid product 13 so obtained was recrystallized from a mixture (9:1) of CH₂Cl₂ and hexane. White solid, Yield 75%; m.pt 129-131° C.; IR (KBr) vₘₐₓ:3357, 2971, 1621, 1541 cm⁻¹; ¹H NMR (600 MHz, MeOD): δ_{H} 1.90 (s, 2H, —H₂C—NH₂), 2.83 (m, 4H, —H₂C—H₂C—NH₂), 3.68 (s, 3H, H₃C—O—), 6.59 (d, 2H, J=7.8 Hz, c″), 6.82 (d, 2H, J=7.8 Hz, b″), 7.27 (m, 8H, b, b′, c′, d, d′), 7.43 (dd, 2H, J=7.8 Hz, c); ¹³C NMR (150 MHz, MeOD): δ_{C} 34.4, 38.6, 54.1, 112.6, 126.5, 126.8, 128.0, 128.3, 128.7, 129.3, 129.9, 130.1, 131.3, 134.1, 134.4, 158.2. MS m/z: 330 (M⁺). Analysis calculated for C₂₃H₂₃NO: C, 83.85; H, 7.04; N, 4.25. Found: C, 83.88; H, 7.02; N, 4.22.

### (Z)-Phenyl-4-(4-methoxyphenyl)-3,4-diphenylbut-3-enylcarbamate (14)

To a stirred suspension of compound 13 (1 mmol) and K₂CO₃ (3 mmol), in dry dioxane at 0° C., was added phenyl chloroformate (1.02 mmol). The reaction mixture was allowed to warm to room temperature and stirred for 2 h. Upon completion of reaction, as evidenced by TLC, K₂CO₃ was filtered off and the filtrate washed with water and dried over anhydrous sodium sulphate. The solvent was removed under vacuo and the solid product 14 so obtained was recrystallized from a mixture (1:9) of CH₂Cl₂ and hexane. White solid, Yield 70%; m.pt 134-136° C.; IR (KBr) vₘₐₓ: 3382, 1703, 1607 1509 cm⁻¹; ¹H NMR (500 MHz, CDCl₃): δ_{H} 2.76 (t, 2H, J=7.0 Hz, —CH₂—CH₂—), 3.28 (t, 2H, J=7.0 Hz, H₂C—H₂C—NH—), 3.71 (s, 3H, —H₃C—O—), 4.86 (b, 1H, —NH), 6.59 (d, 2H, J=8.5 Hz, c″), 6.83 (d, 2H, J=8.5 Hz, b″), 7.23 (m, 15H, Aromatic); ¹³C NMR (125 MHz, CDCl₃): δ_{C} 35.9, 40.3, 55.0, 112.9, 121.6, 125.3, 126.6, 126.9, 128.3, 128.4, 129.2, 129.3, 129.5, 129.6, 131.7, 134.8, 135.9, 141.5, 141.8, 143.1, 151.0, 154.3, 157.8. MS m/z: 450 (M⁺). Analysis calculated for C₃₀H₂₇NO₃: C, 80.15; H, 6.05; N, 3.12. Found: C, 80.10; H, 6.00; N, 3.08.

### (Z)-Ethyl-2-(4-(4-methoxyphenyl)-3,4-diphenylbut-3-enylamino)-2-oxoacetate (15)

To a stirring suspension of 13 (1 mmol) and K₂CO₃ (3 mmol), in dry dioxane at 0° C., was added ethyl oxalyl chloride (1.02 mmol). The reaction mixture was allowed to warm to room temperature and stirred for 2 h. Upon completion of reaction, as evidenced by TLC, K₂CO₃ was filtered off and the filtrate washed with water and dried over anhydrous sodium sulphate. The solvent was removed under vacuo and the solid product 7 so obtained was recrystallized from a mixture (1:9) of CH₂Cl₂ and hexane. White solid, Yield 82%; m.pt 121-123° C.; IR (KBr) vₘₐₓ: 3316, 2961, 1738, 1677, 1606, 1553 cm⁻¹; ¹H NMR (500 MHz, CDCl₃): δ_{H} 1.38 (t, 3H, J=7.0 Hz, —O—CH₂—CH₃), 2.73 (t, 2H, J=7.0 Hz, —CH₂—CH₂—), 3.35 (m, 2H, J=7.0 Hz, H₂C—H₂C—NH—), 3.70 (s, 3H, —H₃C—O—), 4.32 (q, 2H, —O—CH₂—CH₃), 6.59 (d, 2H, J=7.0 Hz, c″), 6.82 (d, 2H, J=7.0 Hz, b″), 6.93 (b, 1H, —NH), 7.25 (m, 10H, Aromatic); ¹³C NMR (125 MHz, CDCl₃): δ_{C} 14.0, 35.0, 39.1, 55.0, 63.1, 112.9, 126.7, 127.0, 127.5, 128.4, 128.5, 129.2, 129.4, 130.4, 130.5, 131.6, 134.6, 135.5, 141.5, 141.7, 143.0, 156.3, 157.8, 160.6. MS m/z: 430 (M⁺). Analysis calculated for C₂₇H₂₇NO₄: C, 75.50; H, 6.34; N, 3.26. Found: C, 75.55; H, 6.30; N, 3.28.

### General Procedure for the Synthesis of Compounds 16-26

To a well stirred solution of 14 (1 mmol) (for 16-21) and 15 (1 mmol) (for 22-26) in dry DMF, was added primary/secondary amine (1.02 mmol) dropwise. The resulting reaction mixture was stirred at 80° C. for 6 h. The progress of the reaction was monitored by TLC and on completion, the reaction mass was poured over ice and extracted with ethyl acetate. The organic extract was washed with brine and water and dried over anhydrous sodium sulphate. The removal of solvent under reduced pressure resulted in an oily mass which was recrystallized from a mixture (1:9) of CH₂Cl₂ and hexane.

### (Z)-1-Benzyl-3-(4-(4-methoxyphenyl)-3, 4-diphenylbut-3-enyl) urea (16)

White solid, Yield 80%; m.pt 158-160° C.; IR (KBr) vₘₐₓ: 3424, 1629, 1606, 1572 cm⁻¹; ¹H NMR (500 MHz, CDCl₃): δ_{H} 2.67 (t, 2H, J=7.0 Hz, —CH₂—CH₂—), 3.19 (t, 2H, J=7.5 Hz, —CH₂—NH—), 3.70 (s, 3H, —H₃C—O—), 4.26 (s, 2H, —NH—CH₂-Ph), 6.57 (d, 2H, J=7.0 Hz, c″), 6.80 (d, 2H, J=7.0 Hz, b″), 7.26 (m, 15H, Aromatic and —NH); ¹³C NMR (125 MHz, CDCl₃): δ_{C} 36.2, 40.1, 44.5, 55.0, 112.9, 126.9, 127.3, 127.5, 128.3, 128.4, 129.3, 129.5, 131.7, 134.8, 136.1, 139.0, 141.2, 142.0, 143.2, 157.8. MS m/z: 463 (M⁺). Analysis calculated for C₃₁H₃₀N₂O₂: C, 80.49; H, 6.54; N, 6.06. Found: C, 80.51; H, 6.57; N, 6.10.

### (Z)-1-Cyclohexyl-3-(4-(4-methoxyphenyl)-3, 4-diphenylbut-3-enyl) urea (17)

White solid, Yield 85%; m.pt 168-170° C.; IR (KBr) vₘₐₓ: 3429, 2929, 1622, 1570, 1508 cm⁻¹; ¹H NMR (500 MHz, CDCl₃): δ_{H} 1.03 (m, 2H, H_{b}), 1.15 (m, 1H, Hf), 1.33 (m, 2H, H_{d}), 1.62 (m, 1H, Hₑ), 1.68 (m, 2H, Hₑ), 1.86 (m, 2H, Hₐ), 2.68 (t, 2H, J=7.0 Hz, —CH₂—CH₂—), 3.19 (t, 2H, J=7.5 Hz, H₂C—H₂C—NH—), 3.70 (s, 3H, —H₃C—O—), 4.32 (dd, 2H, —O—CH₂—CH₃), 6.58 (d, 2H, J=7.0 Hz, c″), 6.81 (d, 2H, J=7.0 Hz, b″), 7.27 (m, 12H, Aromatic and —NH); ¹³C NMR (125 MHz, CDCl₃): δ_{C} 24.9, 25.6, 33.9, 36.2, 40.0, 49.1, 55.0, 112.9, 126.2, 126.9, 128.3, 129.3, 129.5, 131.7, 134.9, 136.3, 141.1, 142.1, 143.2, 157.1, 157.7. MS m/z: 455 (M⁺). Analysis calculated for C₃₀H₃₄N₂O₂: C, 79.26; H, 7.54; N, 6.16. Found: C, 79.30; H, 7.50; N, 6.13.

### (Z)—N-(4-(4-Methoxyphenyl)-3,4-diphenylbut-3-enyl)morpholine-4-carboxamide (18)

White solid, Yield 77%; m.pt 139-141° C.; IR (KBr) vₘₐₓ: 3356, 2960, 1618, 1531, 1507 cm⁻¹; ¹H NMR (500 MHz, CDCl₃): δ_{H} 2.71 (t, 2H, J=7.0 Hz, —CH₂—CH₂—), 3.06 (t, 4H, —CH₂—N—CH₂—), 3.37 (t, 2H, J=7.0 Hz, H₂C—H₂C—NH—), 3.58 (t, 4H, —CH₂—O—CH₂—), 3.70 (s, 3H, —H₃C—O—), 6.58 (d, 2H, J=7.0 Hz, c″), 6.79 (d, 2H, J=7.0 Hz, b″), 7.27 (m, 11H, Aromatic and —NH); ¹³C NMR (125 MHz, CDCl₃): δ_{C} 35.8, 41.3, 43.7, 55.0, 66.4, 112.9, 126.4, 126.9, 128.3, 128.4, 129.4, 129.6, 131.8, 134.8, 136.8, 141.2, 142.9, 143.2, 157.3, 157.8. MS m/z: 443 (M⁺). Analysis calculated for C₂₈H₃₀N₂O₃: C, 75.99; H, 6.83; N, 6.33. Found: C, 75.96; H, 6.87; N, 6.30.

### (Z)-1,1-Diethyl-3-(4-(4-methoxyphenyl)-3,4-diphenylbut-3-enyl)urea (19)

White solid, Yield 80%; m.pt 159-160° C.; IR (KBr) vₘₐₓ: 3356, 2975, 1621, 1603, 1541, 1508 cm⁻¹; ¹H NMR (500 MHz, CDCl₃): δ_{H} 1.05 (t, 6H, J=7.0 Hz, (CH₂)₃), 2.72 (t, 2H, J=7.0 Hz, —CH₂—CH₂—), 3.08 (t, 4H, J=7.0 Hz, —CH₂—N—CH₂—), 3.33 (m, 2H, J=7.0 Hz, —H₂C—H₂C—NH—), 3.70 (s, 3H, —H₃C—O—), 6.58 (d, 2H, J=7.0 Hz, c″), 6.80 (d, 2H, J=7.0 Hz, b″), 7.26 (m, 11H, Aromatic and —NH); ¹³C NMR (125 MHz, CDCl₃): δ_{C} 13.8, 35.9, 40.6, 41.1, 55.0, 112.9, 126.4, 126.8, 128.3, 129.4, 129.5, 131.8, 135.1, 137.0, 141.0, 142.5, 143.2, 156.9, 157.7. MS m/z: 429 (M⁺). Analysis calculated for C₂₈H₃₂N₂O₂: C, 78.47; H, 7.53; N, 6.54. Found: C, 78.49; H, 7.55; N, 6.51.

### (Z)—N-(4-(4-Methoxyphenyl)-3,4-diphenylbut-3-enyl)piperidine-1-carboxamide (20)

White solid, Yield 90%; m.pt 197-198° C.; IR (KBr) vₘₐₓ: 3451, 2943, 1640, 1605, 1519, 1508 cm⁻¹; ¹H NMR (500 MHz, CDCl₃): δ_{H} 1.49 (m, 6H, (CH₂)₃), 2.70 (t, 2H, J=7.0 Hz, —CH₂—CH₂—), 3.08 (m, 4H, —CH₂—N—CH₂—), 3.33 (t, 2H, J=7.0 Hz, —H₂C—H₂C—NH—), 3.70 (s, 3H, —H₃C—O—), 6.58 (d, 2H, J=7.0 Hz, c″), 6.81 (d, 2H, J=7.0 Hz, b″), 7.26 (m, 11H, Aromatic and —NH); ¹³C NMR (125 MHz, CDCl₃): δ_{C} 24.3, 25.5, 35.9, 41.0, 44.7, 55.0, 112.9, 126.4, 126.8, 129.4, 129.6, 131.8, 135.0, 137.0, 140.9, 142.7, 156.5, 157.7. MS m/z: 441 (M). Analysis calculated for C₂₉H₃₂N₂O₂: C, 79.06; H, 7.32; N, 6.36. Found: C, 79.08; H, 7.35; N, 6.31.

### (Z)—N-(4-(4-Methoxyphenyl)-3,4-diphenylbut-3-enyl)pyrrolidine-1-carboxamide (21)

White solid, Yield 78%; m.pt 194.2-195.4° C.; IR (KBr) vₘₐₓ: 3435, 2974, 1636, 1605, 1526, 1508 cm⁻¹; ¹H NMR (500 MHz, CDCl₃): δ_{H} 1.82 (m, 6H, (CH₂)₃), 2.71 (t, 2H, J=7.5 Hz, —CH₂—CH₂—), 3.11 (m, 4H, —CH₂—N—CH₂—), 3.34 (t, 2H, J=7.0 Hz, —H₂C—H₂C—NH—), 3.70 (s, 3H, —H₃C—O—), 6.58 (d, 2H, J=7.5 Hz, c″), 6.80 (d, 2H, J=6.5 Hz, b″), 7.27 (m, 11H, Aromatic and —NH); ¹³C NMR (125 MHz, CDCl₃): δ_{C} 25.5, 36.2, 40.6, 45.2, 55.0, 112.9, 126.3, 126.8, 128.2, 128.3, 129.4, 129.6, 131.8, 135.0, 137.0, 140.9, 142.7, 156.5, 157.7. MS m/z: 427 (M⁺). Analysis calculated for C₂₈H₃₀N₂O₂: C, 78.84; H, 7.09; N, 6.57. Found: C, 78.88; H, 7.05; N, 6.55.

### (Z)—N1-Benzyl-N2-(4-(4-methoxyphenyl)-3,4-diphenylbut-3-enyl)oxalamide (22)

White solid, Yield 82%; m.pt 169-172° C.; IR (KBr) vₘₐₓ: 3313, 2931, 1653, 1509 cm⁻¹; ¹H NMR (500 MHz, CDCl₃): δ_{H} 2.72 (dd, 2H, J=7.0 Hz, —CH₂—CH₂—), 3.30 (2×t, 2H, J=6.5 Hz, —H₂C—H₂C—NH—), 3.70 (s, 3H, —H₃C—O—), 4.50 (m ABq, 2H, J=6.5 Hz, —NH—CH₂-Ph), 6.58 (d, 2H, J=6.5 Hz, c″), 6.82 (d, 2H, J=6.5 Hz, b″), 7.26 (m, 16H, Aromatic and —NH), 7.70 (b, 1H, —NH); ¹³C NMR (125 MHz, CDCl₃): δ_{C} 35.2, 38.6, 43.7, 53.4, 55.0, 112.9, 126.6, 126.9, 127.8, 128.3, 128.5, 128.8, 129.2, 129.5, 131.6, 134.6, 135.5, 136.8, 141.3, 141.7, 143.0, 157.8, 159.4, 159.7. MS m/z: 491 (M⁺). Analysis calculated for C₃₂H₃₀N₂O₃: C, 78.34; H, 6.16; N, 5.71. Found: C, 78.39; H, 6.11; N, 5.65.

### (Z)—N1-Cyclohexyl-N2-(4-(4-methoxyphenyl)-3,4-diphenylbut-3-enyl)oxalamide (23)

White solid, Yield 90%; m.pt 198-199° C.; IR (KBr) vₘₐₓ: 3310, 2934, 1651, 1509 cm⁻¹; ¹H NMR (500 MHz, CDCl₃): δ_{H} 1.25 (m, 3H, H_{b}, H_{f}), 1.40 (m, 2H, H_{d}), 1.65 (m, 2H, Hₑ and —NH), 1.76 (m, 2H, Hₑ), 1.93 (m, 2H, Hₐ), 2.71 (dd, 2H, J=7.0 Hz, —CH₂—CH₂—), 3.28 (2×t, 2H, J=6.5 Hz, —H₂C—H₂C—NH—), 3.70 (s, 3H, —H₃C—O—), 6.58 (d, 2H, J=7.0 Hz, c″), 6.83 (d, 2H, J=7.0 Hz, b″), 7.26 (m, 11H, Aromatic and —NH); ¹³C NMR (125 MHz, CDCl₃): δ_{C} 24.7, 25.3, 32.6, 35.1, 38.5, 48.7, 55.0, 112.9, 126.6, 126.9, 128.3, 128.4, 129.2, 129.5, 131.6, 134.6, 135.5, 141.3, 141.7, 143.0, 157.8, 158.7, 159.8. MS m/z: 483 (M⁺). Analysis calculated for C₃₁H₃₄N₂O₃: C, 77.15; H, 7.10; N, 5.80. Found: C, 77.19; H, 7.05; N, 5.76.

### (Z)—N-(4-(4-Methoxyphenyl)-3,4-diphenylbut-3-enyl)-2-oxo-2-(pyrrolidin-1-yl)acetamide (24)

White solid, Yield 80%; m.pt 151-152° C.; IR (KBr) vₘₐₓ: 3315, 2973, 1682, 1622, 1508 cm⁻¹; ¹H NMR (500 MHz, CDCl₃): δ_{H} 1.60 (b, 1H, —NH), 1.82 (m, 4H, —(CH₂)₂), 1.96 (m, 2H, —CH₂), 2.71 (dd, 2H, J=7.0, 7.5 Hz, —CH₂—CH₂—), 3.27 (2×t, 2H, J=7.0 Hz, —H₂C—H₂C—NH—), 3.55 (t, 2H, J=7.0 Hz, H₂C—N—CH₂—), 3.70 (s, 3H, —H₃C—O—), 3.96 (t, 2H, H₂C—N—CH₂), 6.58 (d, 2H, J=7.0 Hz, c″), 6.82 (d, 2H, J=7.0 Hz, b″), 7.27 (m, 10H, Aromatic and —NH), 7.47 (b, 1H, —NH); ¹³C NMR (125 MHz, CDCl₃): δ_{C} 23.4, 26.8, 35.2, 38.1, 47.9, 48.6, 55.0, 112.9, 126.5, 126.8, 128.2, 128.4, 129.3, 129.5, 131.7, 134.8, 135.8, 141.5, 143.1, 157.8, 159.2, 160.3. MS m/z: 455 (M⁺). Analysis calculated for C₂₉H₃₀N₂O₃: C, 76.63; H, 6.65; N, 6.16. Found: C, 76.60; H, 6.68; N, 6.11.

### (Z)—N-(4-(4-Methoxyphenyl)-3,4-diphenylbut-3-enyl)-2-morpholino-2-oxoacetamide (25)

White solid, Yield 68%; m.pt 118.4-119.3° C.; IR (KBr) vₘₐₓ: 3319, 2973, 1680, 1625, 1509 cm⁻¹; ¹H NMR (500 MHz, CDCl₃): δ_{H} 1.65 (b, 1H, —NH), 2.71 (dd, 2H, J=7.0, 7.0 Hz, —CH₂—CH₂—), 3.27 (2×t, 2H, J=6.5 Hz, —H₂C—H₂C—NH—), 3.65 (m, 2H, J=7.0 Hz, H₂C—N—CH₂—), 3.70 (s, 3H, —H₃C—O—), 3.71 (m, 2H, H₂C—O—CH₂—), 3.74 (m, 2H, —H₂CO—CH₂—), 4.18 (m, 2H, H₂C—N—CH₂—), 6.57 (d, 2H, J=7.0 Hz, c″), 6.81 (d, 2H, J=7.0 Hz, b″), 7.27 (m, 10H, Aromatic); ¹³C NMR (125 MHz, CDCl₃): δ_{C} 35.1, 38.3, 43.6, 47.0, 55.0, 66.8, 67.3, 112.9, 126.6, 126.9, 128.3, 128.4, 129.2, 129.4, 131.7, 134.7, 135.6, 141.5, 141.6, 143.1, 157.8, 160.4, 160.5. MS m/z: 471 (M⁺). Analysis calculated for C₂₉H₃₀N₂O₄: C, 74.02; H, 6.43; N, 5.95. Found: C, 74.05; H, 6.40; N, 5.91.

### (Z)—N-(4-(4-Methoxyphenyl)-3,4-diphenylbut-3-enyl)-2-oxo-2-(piperidin-1-yl)acetamide (26)

White solid, Yield 85%; m.pt 125-127° C.; IR (KBr) vₘₐₓ: 3294, 2937, 1674, 1621, 1508 cm⁻¹; ¹H NMR (500 MHz, CDCl₃): δ_{H} 1.64 (m, 6H, —(CH₂)₃), 2.71 (dd, 2H, J=7.0, 7.5 Hz, —CH₂—CH₂—), 3.28 (2×t, 2H, J=6.5 Hz, —H₂C—H₂C—NH—), 3.58 (m, 2H, J=7.0 Hz, —H₂C—N—CH₂—), 3.70 (s, 3H, —H₃C—O—), 3.95 (m, 2H, H₂C—N—CH₂—), 6.58 (d, 2H, J=7.0 Hz, c″), 6.83 (d, 2H, J=7.0 Hz, b″), 7.04 (b, 1H, —NH), 7.26 (m, 10H, Aromatic); ¹³C NMR (125 MHz, CDCl₃): δ_{C} 24.5, 25.7, 26.8, 35.2, 38.2, 44.4, 47.4, 55.0, 112.9, 126.5, 126.9, 128.3, 128.4, 129.3, 129.5, 131.7, 134.8, 135.8, 141.4, 141.5, 143.1, 157.8, 160.9, 161.4. MS m/z: 469 (M⁺). Analysis calculated for C₃₀H₃₂N₂O₃: C, 76.90; H, 6.88; N, 5.98. Found: C, 76.85; H, 6.85; N, 5.94.

### Cell Lines

Human breast cancer cell lines MCF-7 and MDA-MB-231 were obtained from the American Type Culture Collection (Manassas, Va.). Breast cancer cells were cultured in DMEM supplemented with 10% FBS and antibiotics. The cells were maintained at 37° C. in a humidified atmosphere of 5% CO₂. The cells were routinely screened for mycoplasma using Hoechst 33258 staining.

### Cell Viability Assays

The effects of novel triarylethylene derivatives on the cell viability were determined by the MTT uptake method using previously described methods (Kaur et al., 2014, Eur. J. Med. Chem. 86:211-218). Briefly, 3000 cells were incubated with various concentrations of compounds in triplicate in a 96-well plate for 72 h at 37° C. An MTT solution was added to each well and incubated for 3 h at 37° C. After 3 h, DMSO was added and the optical density was measured at 570 nm using a 96-well multiscanner (Dynex Technologies, MRX Revelation; Chantilly, Va., USA). Backgrounds were subtracted at 630 nm. IC₅₀ values were calculated by non-linear regression analysis using Prism software.

### Western Blot Analysis

To determine the effect of novel triarylethylene derivatives on proteins involved in metastasis, and adhesion, whole-cell extracts were prepared by subjecting cells to lysis in RIPA buffer supplemented with protease and phosphatase inhibitor cocktails using previously described methods (Pandey et al., 2014, Exp. Hematol. 42:883-896). Lysates were spun at 15,000 rpm for 10 minutes to remove insoluble material. Supernatant were collected and kept at −80° C. Lysates were resolved by SDS-PAGE. After electrophoresis, the proteins were electro-transferred to PVDF membranes, blotted with the relevant antibodies, and detected by enhanced chemo-luminescence reagent.

### Scratch Assays

Human breast cancer MDA-MB-231 cells were seeded in 6-well culture plates and grown in complete medium (DMEM with 10% FBS). Once cells were completely confluent uniform vertical and horizontal scratches were made through the cell monolayers. After scratches, cells were washed gently with phosphate buffer saline (PBS) and images were captured. Cells were treated with Ospemifene and compounds of the invention (13, 22, and 23) or DMSO in complete media. Cells were then allowed to migrate for 24 h and images were captured. The widths of scratches were measured at five different locations with AxioVision software (AxioVision Inc.). The mean percentage of migration of each treatment was calculated and normalized to that of vehicle (DMSO) control.

### In Vitro Invasion Assays

Human breast cancer MDA-MB-231 cells were serum starved for 24 h in serum-free medium (DMEM without FBS). After starving 50,000 cells were transferred onto Matrigel pre-coated invasion inserts (upper chamber, BD Biocoat). Lower chambers of plate were filled with complete medium (DMEM and 10% FBS) and cells were allowed to migrate under chemotaxis. After 4 h, cells growing in upper chambers were treated with compound 13 and allowed to invade for 24 h. After incubation cells that had invaded to the opposite side of the Transwell membrane were washed with PBS and fixed in chilled 70% ethanol for 15 min and air dried. Cells were stained with 0.5% crystal violet for 10 min. Cells were washed, dried, and images were captured at 40× magnification.

### Docking Studies

The crystal structures of ERα (1GWR) and ERβ (1QKM) co-crystallized with native inhibitors were downloaded from the protein data bank (www.rcsb.org). No associated water molecules and inhibitors were considered in docking and were eliminated from both proteins using DS. The protonation states of amino acids of both proteins were determined at physiological pH and partial charges to all atoms were assigned with CHARMm force field (FF), using Prepare algorithm in DS. In order to remove the bad contacts, both proteins were minimized using conjugated gradient method in DS. Different conformations of both compounds (13 and 25) were generated using “Generate Conformation” protocol embedded in the DS. Total number of conformations sampled for compound 13 and 25 were 11 and 85 respectively, which were further geometrically optimized using “Minimize Ligands” module and the lowest energy conformation for each compound was selected for docking. Before docking, a binding sphere covering all active site residues was generated for each protein using the Define and Edit binding site module. Docking of compounds was performed using the CDocker algorithm (Wu et al., 2003, J. Comp. Chem. 24:1549-1562). CDocker is a CHARMm FF based program in which protein is held fixed and the ligand conformational profile is explored by the molecular dynamics method followed by their refinement using grid-based simulated annealing. Different ligand poses obtained from docking were separated based on the scoring function (-CDocker energy), and the best pose was subjected to binding energy calculations.

The results of the experiments are now described.

### Synthetic Chemistry

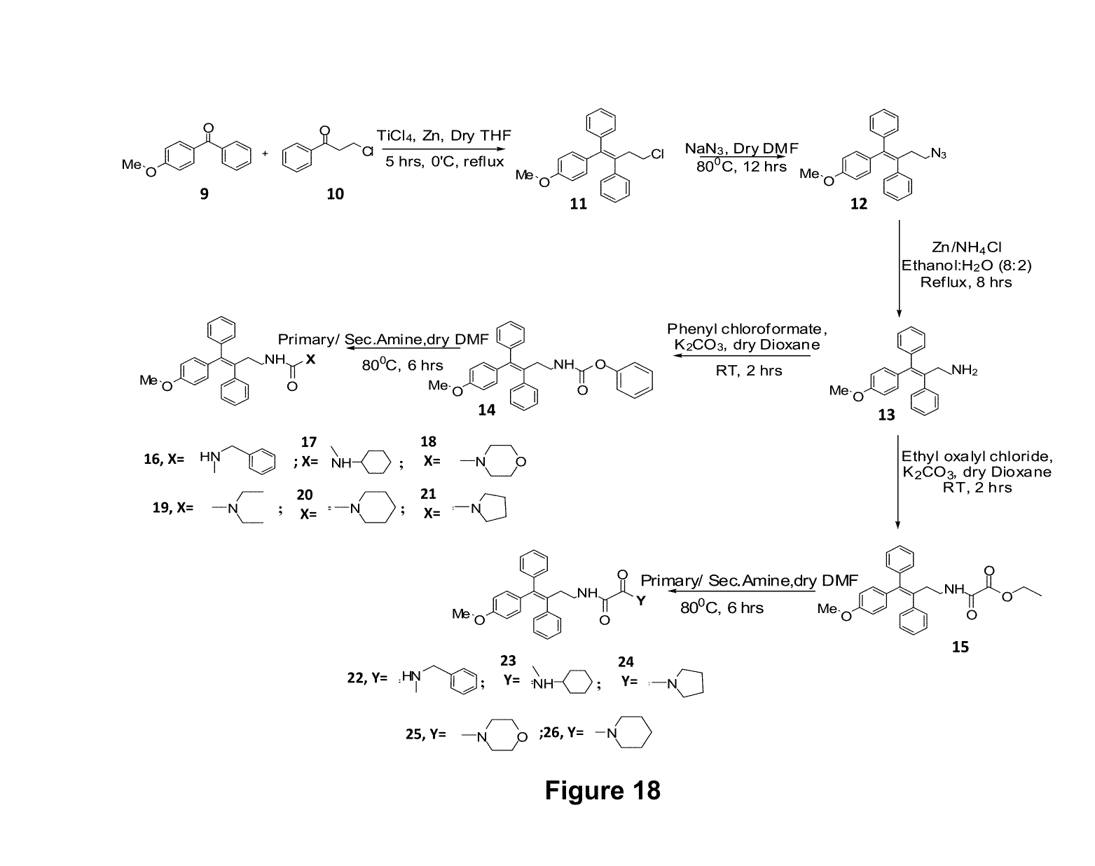

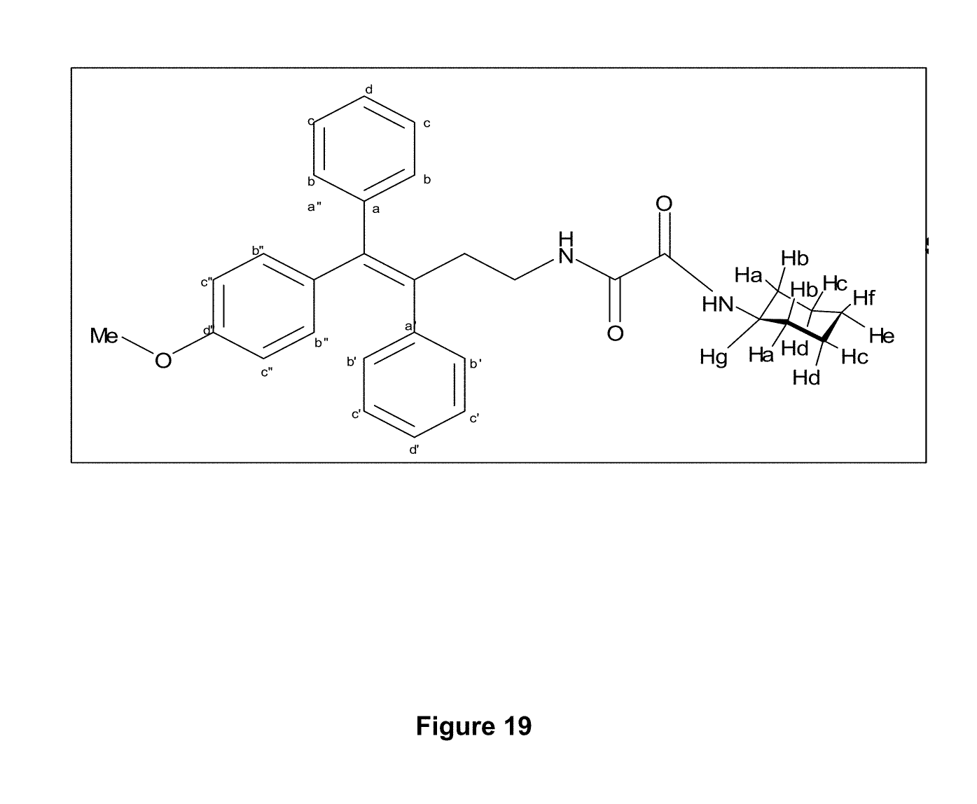

The previously unreported Z-(4-chloro-1-(4-methoxyphenyl)but-1-ene-1,2-diyl)dibenzene (11) required as precursor for the synthesis of desired triarylethylenes (12-26) was synthesised by following the McMurry reaction between p-methoxy benzophenone and 1-chloropropiophenone. The treatment of 11 with 5 mmol of sodium azide in dry DMF at 60° C. resulted in azide 12. The amine 13 was obtained by the treatment of 12 with zinc dust and ammonium chloride in a mixture (4:1) of ethanol and water. The carbamic acid phenyl ester 14 and oxalamic acid ethyl ester 15 were obtained by the treatment of 13 with phenyl chloroformate and ethyl oxalyl chloride, respectively. The novel urea derivatives (16-21) were prepared by refluxing a solution of 14 and primary/secondary amines in dry DMF for 6 h. A similar synthetic protocol was followed for the synthesis of oxalamide derivatives (22-26) as elucidated in FIG. 18. The novel triarylethylene analogs mentioned above were purified and characterized using IR, LCMS, ¹H NMR and ¹³C NMR techniques.

The compounds were evaluated for their anticancer activity and mechanism of action on MCF-7 (ER-positive) and MDA-MB-231 (ER-negative) human breast cancer cell lines. The docking studies were also performed to have a better understanding of the bonding and binding energies of the test compounds with the ER receptors.

### Pharmacology

### Novel Triarylethylene Analogs Showed Differential Cytotoxic Potential in ER-Negative and ER-Positive Cells

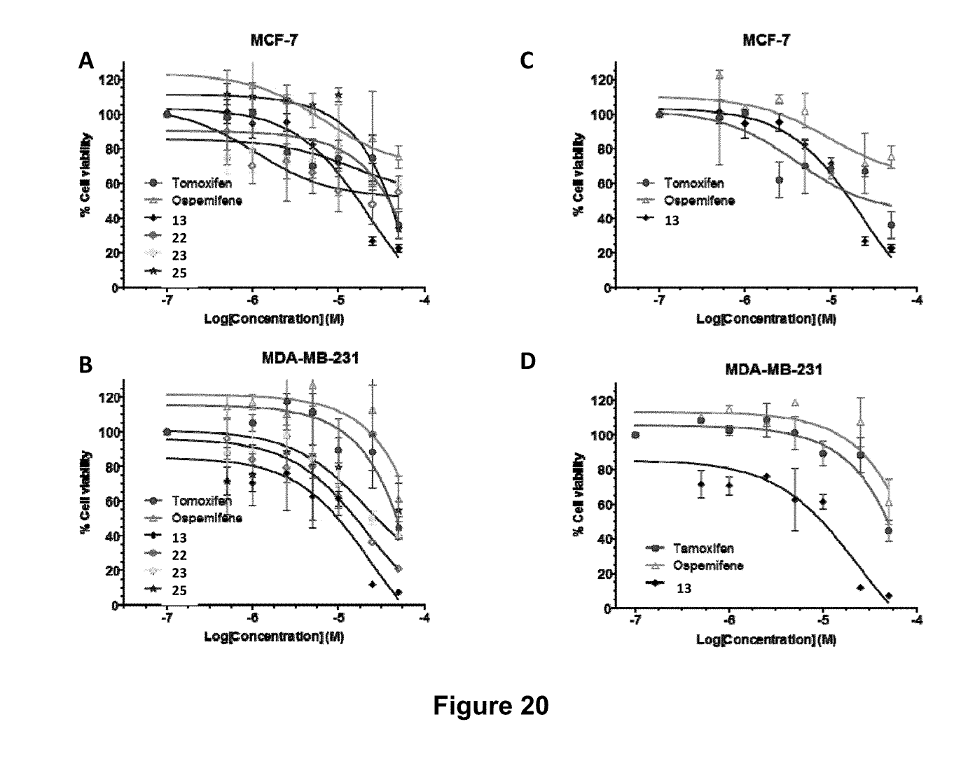

Novel compounds (11-26) having a triarylethylene scaffold were evaluated for their activity against MCF-7 (ER-positive) and MDA-MB-231 (ER-negative) human breast cancer cell lines following MTT assay using previously described methods (Kaur et al., 2014, Eur. J. Med. Chem. 86:211-218). Both ER+ and ER− cell lines were used to evaluate if the novel analogs were selectively cytotoxic to the ER+ cells similar to Ospemifene. The amine 13 and oxalamide 23 exhibited remarkable activity with IC₅₀ values much less than Tamoxifene and Ospemifene against both MCF-7 and MDA-MB-231 cell lines while the oxalamide 22 was selectively cytotoxic to non-estrogen dependent MDA-MB-231 (ER− negative) cells. Compounds 11-12, 14-21 and 24-26 were relatively less effective against both the cell lines. Although not wishing to be bound by any particular theory, these results suggest that the replacement of chloro 11 with azide 12 and conversion of amine 13 to amides 14 and 15 & ureas 16-21 proved to be ineffective in improving the anticancer activity against two studied cell lines. However, the replacement of the chloro group 11 with amino group 13 and its conversion to oxalamides 22 and 23, via the reaction of ester 15 with primary aliphatic amines, resulted in significant enhancement of cytotoxicity. On the other hand, the oxalamides 24-26 obtained through the reactions of 15 with secondary amines proved to be relatively ineffective, although compound 25 exhibited good activity against MDA-MB-231 cells. These results support the hypothesis that the replacement/shortening of O-ethyl amino and O-hydroxyl ethyl chains of Tamoxifen and Ospemifene with O-methyl had no significant effect on anti-breast cancer activity of the studied compounds and that the presence of amino group and the oxalamido group that forms primary aliphatic amines, played a predominant role in activity enhancement. Overall, compounds 13, 22, 23 and 25 exhibited significant enhancement of anticancer activities against MCF-7 (ER-positive) and MDA-MB-231 (ER-negative) human breast cancer cell lines (Table 3). Dose-response curves for data of compounds 13, 22, 23 and 25 in comparison to controls Ospemifene and Tamoxifen on both MCF-7 and MDA-MB-231 cell lines are represented by a nonlinear regression plot as depicted in FIG. 20.

TABLE 3
Cytotoxicity of compounds of the invention in breast cancer cells

| | MDA-MB-231 | MCF-7 |
|---|---|---|
| Analogs | (IC₅₀, μM) | (IC₅₀, μM) |
| 13 | 11.4 ± 4.2 | 16.9 ± 7.7 |
| 15 | >50 | >50 |
| 16 | >50 | >50 |
| 17 | >50 | 37.2 ± 13.7 |
| 18 | 42.8 ± 12.2 | >50 |
| 19 | 40.7 ± 12.2 | >50 |
| 20 | >50 | >50 |
| 21 | >50 | >50 |
| 22 | 11.5 ± 3.8 | >50 |
| 23 | 12.2 ± 5.3 | 12.1 ± 4.5 |
| 24 | 48.6 ± 14.5 | >50 |
| 25 | 20.0 ± 9.8 | 43.4 ± 7.5 |
| 26 | >50 | >50 |

ER-negative and - positive breast cancer cells were treated with compounds for 72 h and IC₅₀ was determined.

Triarylethylene Derivatives Inhibit Expression of Proteins Associated with Adhesion, Migration and Metastasis

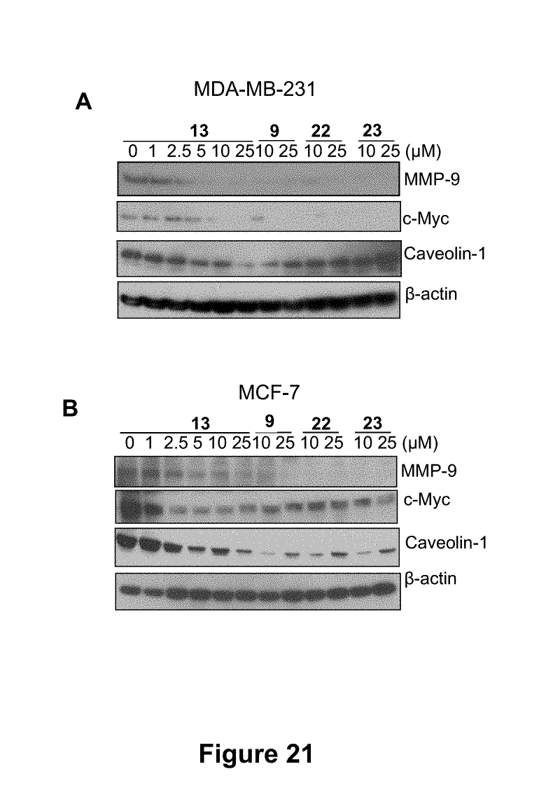

Caveolins are involved in diverse biological functions, including vesicular trafficking, cell adhesion, and apoptosis (Wary et al., 1998, Cell 94:625-634). The matrix metalloproteinases (MMPs) are a family of proteases that target many extracellular proteins including other proteases, cell surface receptors, and adhesion molecules. Among the family members, MMP-9 has been characterized as critical factors for tumor invasion, angiogenesis, and carcinogenesis (Rolli et al., 2003, Proc. Natl. Acad. Sci. USA 100:9482-9487). Similarly, members of the Myc function as transcriptional regulators with roles in various aspects of cells including proliferation (Adhikary and Eilers, 2005, Nat. Rev. Mol. Cell Biol. 6:635-645). The earlier results support the hypothesis that triarylethylene derivatives hold the cytotoxic potential against ER-negative and positive cells (Table 1 and FIG. 20). To further validate whether these analogs possess the anti-invasive and anti-metastatic abilities, the effect of ospemifene derivatives on the expression of MMP-9, c-Myc, and caveolin was investigated because these proteins play critical role in adhesion, angiogenesis, migration, and metastasis. As shown in FIG. 21, it was found that 13 inhibited the expression of MMP-9, c-Myc and caveolin in a dose dependent manner. However the response of 13 in MDA-MB-231 was dramatic as compared to MCF-7 (ER-positive) cells. Although not wishing to be bound by any particular theory, this result suggests the specificity of compound 13 towards ER-negative cells. Other derivatives such as 22 and 23 inhibited the expression of MMP-9 and c-Myc; however, they did not inhibit the expression of caveolin.

### Triarylethylene Derivatives Inhibit Migration of Human Breast Cancer MDA-MB-231 (ER−) Cells

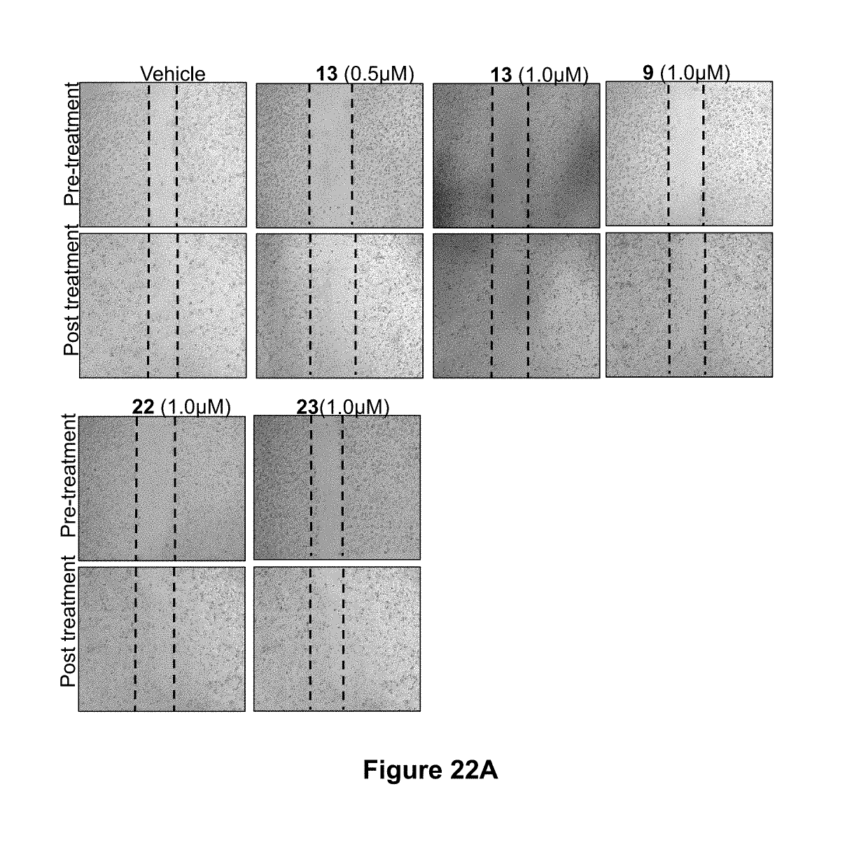

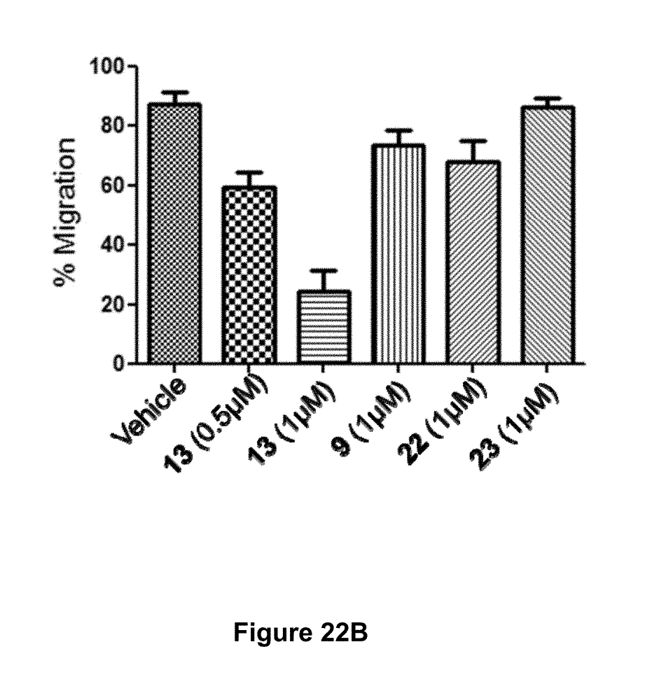

Because ER-negative breast cancers cells are highly metastatic (Dent et al., 2009, Breast Cancer Res. Treat. 115:423-428), anti-metastatic properties of compounds of the invention were further investigated. Breast cancer MDA-MB-231 cells were treated with either Ospemifene or compounds of the invention or DMSO and migration was determined by performing scratch assays. Cells treated with 13 only migrated 60% and 25% at dose of 0.5 and 1 μM, respectively. However other derivatives were found ineffective at 1 μM dose (FIGS. 22A-22B).

### Compound 13 Inhibits Invasion of ER-Cells

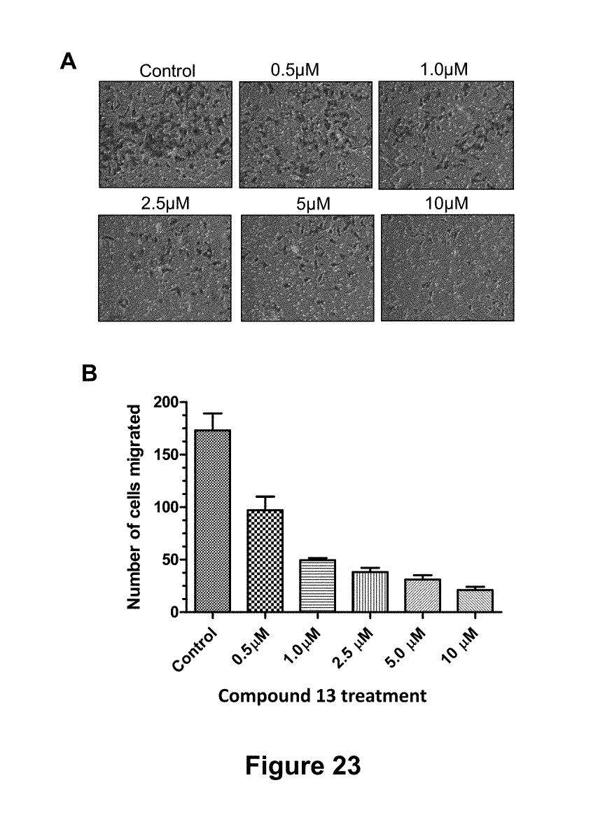

Breast cancer is one of the most metastatic malignancies and unfortunately not many options are available to prevent or cure invasion and metastasis. The potential of compound 13 as anti-metastatic agent was examined. The results showed that compound 13 inhibits the expression of proteins involved in adhesion, migration and metastasis, particularly more effectively in ER-negative MDA-MB-231 cells. To evaluate the effect of 13 on cell invasion, MDA-MB-231 cells were treated with DMSO or different concentrations of 13 and allowed to invade through Matrigel coated membranes (8.0 μm) for 24 h. ER-negative cells treated with 13 migrated only 56-12% compared to their respective DMSO treated controls (FIG. 23A-23B). These results demonstrate that compound 13 possesses anti-metastatic properties.

### Docking Analysis

Docking has recently been employed for several purposes including ligand binding affinity prediction, ligand pose prediction and lead identification (Kitchen et al., 2004, Nat. Rev. Drug Disc. 3:935-949). The utilization of docking method in targeting estrogen receptor (ER) has been very useful for the design of new anti-breast cancer agents due to critical role of this protein in gene expression and transcription. In order to support these experimental results, docking simulations were conducted on representative compounds to illuminate their binding modes in the ligand binding domain (LBD) of ER (both ERα and ERβ). There are twelve α-helices (H1-H12) and a β-hairpin in the LBD, out of which H12 is very important for receptor activation and acts as its switch by adopting a characteristic conformation upon ligand binding (Nettles et al., 2007, EMBO Reports 8:563-568; Pike, Clin. Endocrinology Metabol. 20:1-14). The X-ray co-ordinates of ERα (pdb id: 1GWR) and ERβ (pdb id: 1QKM) co-crystallized with 17β-estradiol and genistein, respectively were downloaded from the protein data bank website (www.rcsb.org), and processed further for docking simulations as described above. The visualization of X-ray structures (using DS visualizer) revealed the interaction of both bound ligands with similar amino acid residues (Arginine, Glutamic acid and Histidine) of the receptors irrespective of their numbering, and was considered for docking in the study.

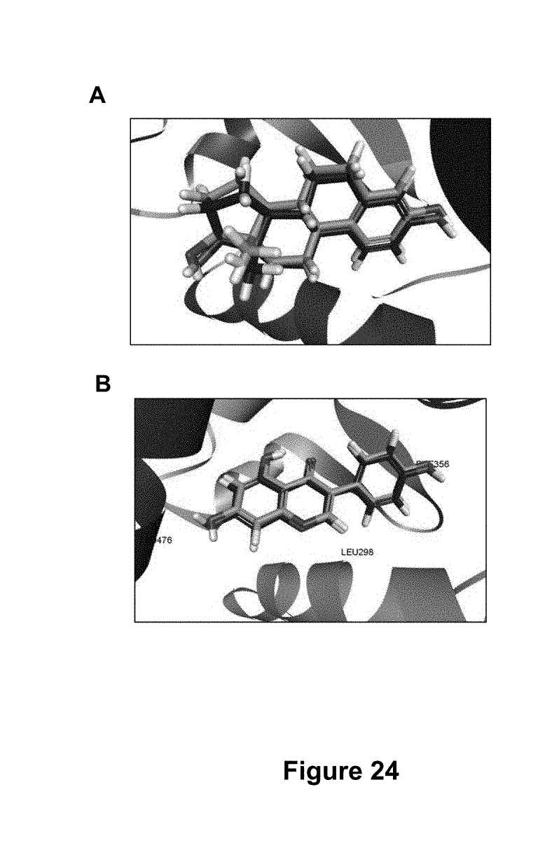

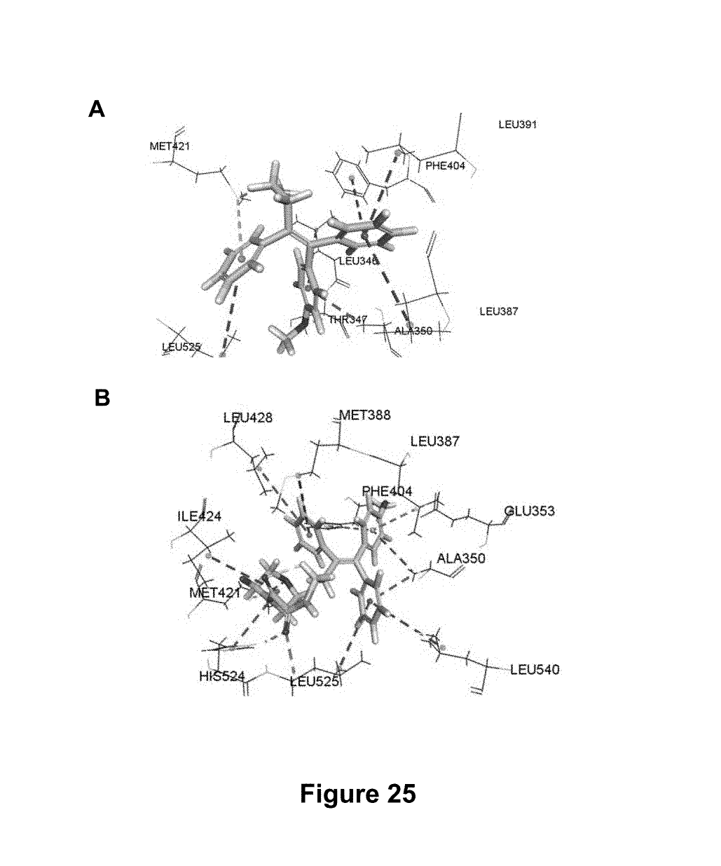

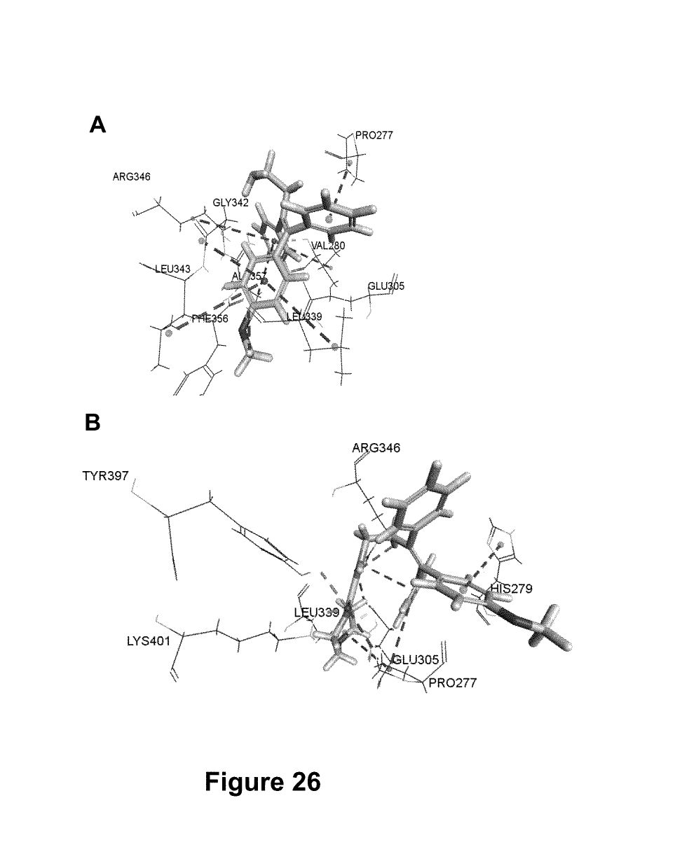

Initially, the re-docking of native inhibitors (estradiol and genistein) in the binding site of both proteins (ERα and ERβ) was performed to check the efficiency of docking procedure, using the CDocker docking algorithm (Wu et al., 2003, J. Comp. Chem. 24:1549-1562) embedded in the Discovery Studio (DS). The best poses of both inhibitors were sampled based on the scoring functions (-CDocker energy), and compared with their X-ray structures using DS visualizer. The most favorable predicted binding conformation of both inhibitors exhibited a good three-dimensional structural correlation relative to their X-ray structures (FIG. 24A-24B) as evidenced by their lower computed root mean square deviations (RMSD<1 Å). Moreover, both inhibitors interacted with similar amino acid residues of the proteins as were observed in their X-ray structures and thus validated the docking protocol. Two representative compounds (13 and 25) were subsequently docked into the binding sites of ERα and ERβ using the same protocol as used for native inhibitors. The computed binding energy (BE) data suggested 13 as a stronger inhibitor of ERa (BE=−65.5 kcalmol⁻¹) compared to 25 (BE=−38.2 kcalmol⁻¹) supporting the higher anti-cancer potency of the former than latter. Compound 13 also exhibited stronger interaction with ERb (BE=−37.0 kcalmol⁻¹) in comparison to its structural analogue 25 (BE=−26.2 kcalmol⁻¹). The complexes of representative compounds with both receptors were further visualized (using DS) to get a deeper understanding of their binding modes in the binding sites, and are pictorially depicted in FIGS. 21-22. Compound 13 (FIG. 25A) also exhibited predominantly hydrophobic interactions (p-alkyl) with ERa residues (Leu525, Ala350, Leu346, Leu391, Leu 387, Thr347) and a characteristic T-shaped π-π stacking interaction with Phe404 through its aromatic rings, clearly supporting the importance of triarylethylene framework in this category of molecules. A single hydrogen bond between —NH₂ moiety (proton donor) of 13 and sulfur atom (proton acceptor) of Met421 was also observed. The binding orientation of 25 (FIG. 25B) revealed the presence of a hydrogen bond with active site residue (His524), and another hydrogen bond interaction (non-conventional) with Leu525 in addition to hydrophobic interactions with Met421, Ile424, Leu428, Met388, Leu387, Ala350, Leu540, Leu525 and Phe404 residues of ERα. Compound 13 interacted with ERβ (FIG. 26A) preferably via hydrophobic interactions (with Arg346, Leu343, Phe356, Glu305, Pro277, Gl342 and Ala357) and hydrogen bonding (with Leu339). Compound 25 formed two conventional hydrogen bonds with Lys401 and Tyr397, one non-conventional hydrogen bond with Pro277 and few hydrophobic interactions with Pro277, His279, Arg346, and Glu305 amino acid residues of the ERb (FIG. 26B). The aromatic rings of the compounds were found to be very significant in stabilizing their complexes with both the receptors.

Novel compounds 11-26 having triarylethylenes scaffold were prepared and screened for their activity against MCF-7 (ER-positive) and MDA-MB-231 (ER-negative) human breast cancer cell lines. Compounds 13 and 23 exhibited remarkable activity with IC₅₀ values much less than Tamoxifene and Ospemifene, used as standards, against both MCF-7 and MDA-MB-231 cell lines while the oxalamide 22 exhibited enhanced activity selectively against MDA-MB-231 cells. Although not wishing to be bound by any particular theory, this data suggested that the presence of an amino or oxalamido substitution on O-methyl analogs led to an increase in potency of triarylethylene analogs. The Western blot analysis to evaluate the expression of proteins associated with adhesion, migration and metastasis, and the scratch assay to evaluate the migration of human breast cancer MDA-MB-231 (ER−) cells, identified compound 13 as a highly effective analog. Compound 13 effectively inhibited the expression of MMP-9, c-Myc and caveolin in a dose dependent manner, particularly in MDA-MB-231 cells. Additionally, it suppressed in vitro wound healing and invasion, clearly demonstrating its anti-metastasis properties. The experimental results were supported by the molecular docking studies with binding energy data showing compound 13 to be a strong inhibitor of ERα and ERβ. In summary, compound 13, having a free amino functionality, exhibited remarkable cytotoxicity against both ER-positive and ER-negative cells and effectively inhibited the migration and invasion of breast cancer cells, and thus may be useful for inhibiting both primary tumor growth and metastatic lesions.

### Example 3: Compound 13 was Found to be More Effective in Treating ER-Negative (ER−) and ER-Positive (ER+) Breast Cancer Cells than Ospemifene and Tamoxifen

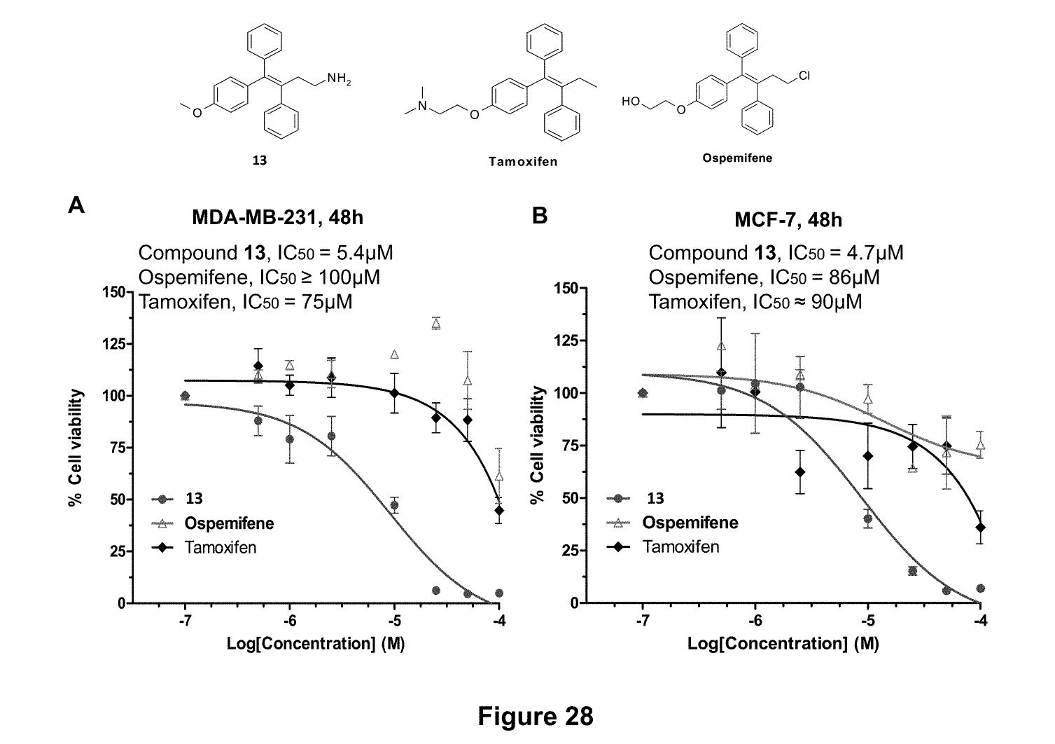

Compound 13 was found to be more effective in treating ER-negative (ER−) and ER-positive (ER+) breast cancer cells than Ospemifene and Tamoxifen (FIG. 28). ER-negative (MDA-MB-231) and ER-positive (MCF-7) breast cancer cells were treated with increasing amounts of compound 13 along with Ospemifene and Tamoxifen (0.5-100 μM) for 48 h. Cell viability was then analyzed by the MTT assay. Cell viability was presented as non-linear regression plot.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of formula (I): 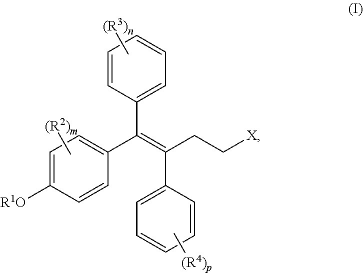
wherein in formula (I):
R¹ is selected from the group consisting of H and alkyl, wherein the alkyl group is optionally substituted;
each occurrence of R², R³, and R⁴ is independently selected from the group consisting of H, —C₁-C₆ alkyl, —C₁-C₆ fluoroalkyl, heteroalkyl, F, Cl, Br, I, —CN, —NO₂, —OR⁴, —SR⁴, —S(═O)R⁴, —S(═O)₂R⁴, —NHS(═O)₂R⁴, —C(NH)(NH₂), —C(═O)R⁴, —OC(═O)R⁴, —CO₂R⁴, —OCO₂R⁴, —CH(R⁴)₂, —N(R⁴)₂, —C(═O)N(R⁴)₂, —OC(═O)N(R⁴)₂, —NHC(═O)NH(R⁴), —NHC(═O)R⁴, —NHC(═O)OR⁴, —C(OH)(R⁴)₂, and —C(NH₂)(O₂;
X is selected from the group consisting of N₃, N(R⁵)(R⁶), Cl, Br, I, and F;
R⁵ and R⁶ are each independently selected from the group consisting of H, —C₁-C₆ alkyl, —C(O)R⁷, and —C(S)R⁷;
R⁷ is selected from the group consisting of OR⁸, N(R⁸)(R⁹), C(O)R⁸, and C(O)N(R⁸)(R⁹);
R⁸ and R⁹ are each independently selected from the group consisting of hydrogen, —C₁-C₆ alkyl, aryl, cycloalkyl, and —C₁-C₆ alkyl-aryl, wherein the alkyl, aryl, cycloalkyl, or alkylaryl group may be optionally substituted, and wherein R⁸ and R⁹ may combine to form a ring, wherein the ring may optionally contain two or more heteroatoms;
m is an integer from 0 to 4;
n is an integer from 0 to 5; and
p is an integer from 0 to 5,
a salt or solvate, and any combinations thereof,
with the proviso that the compound of formula (I) is not

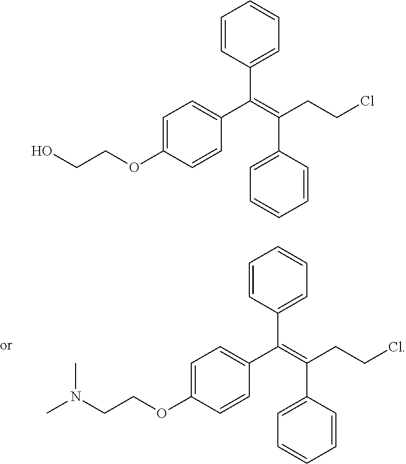

2. The compound of claim 1, wherein R¹ is selected from the group consisting of H, methyl, —(CH₂)₂OH, —(CH₂)₂OS(O)₂CH₃, —(CH₂)₂N₃, —(CH₂)₂NH₂, and —(CH₂)₂N(CH₃)₂.

3. The compound of claim 1, wherein X is N(R⁵)(R⁶).

4. The compound of claim 3, wherein either R⁵ and R⁶ are each H or R⁵ is H and R⁶ is —C(O)R⁷.

5. The compound of claim 1, wherein m is 0, n is 0, and p is 0.

6. The compound of claim 1, wherein the compound is selected from the group consisting of: 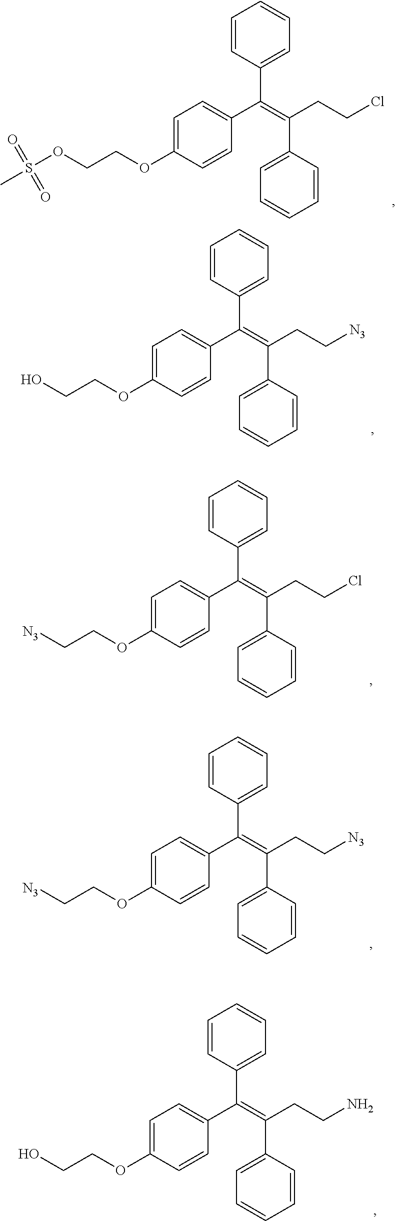 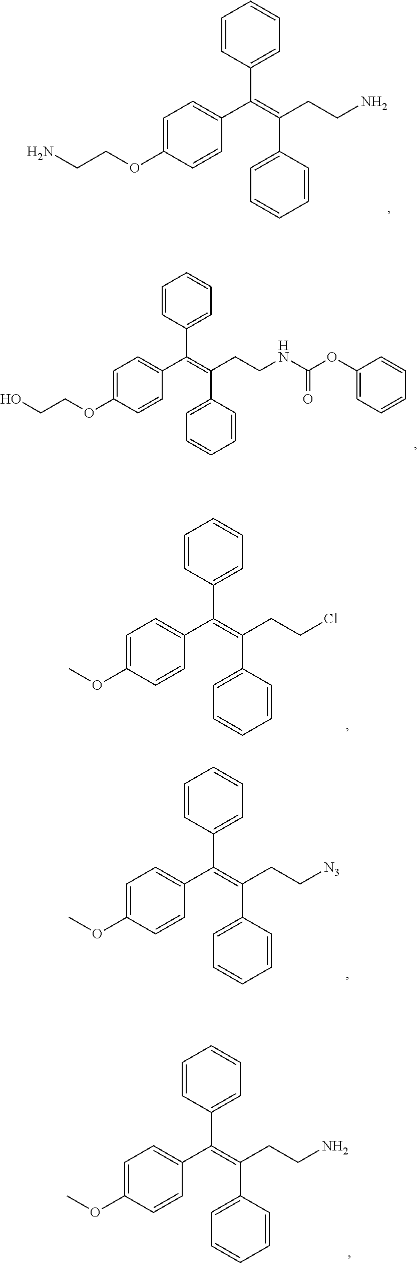 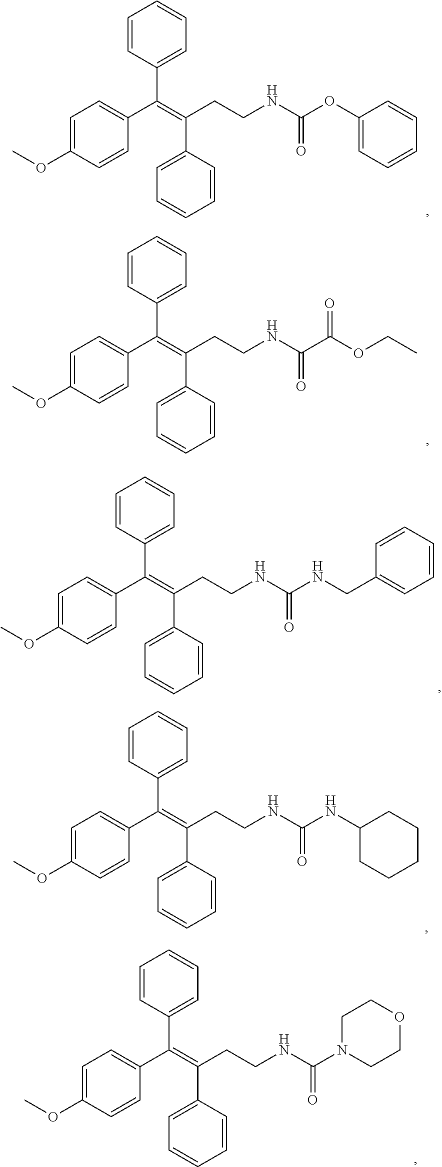 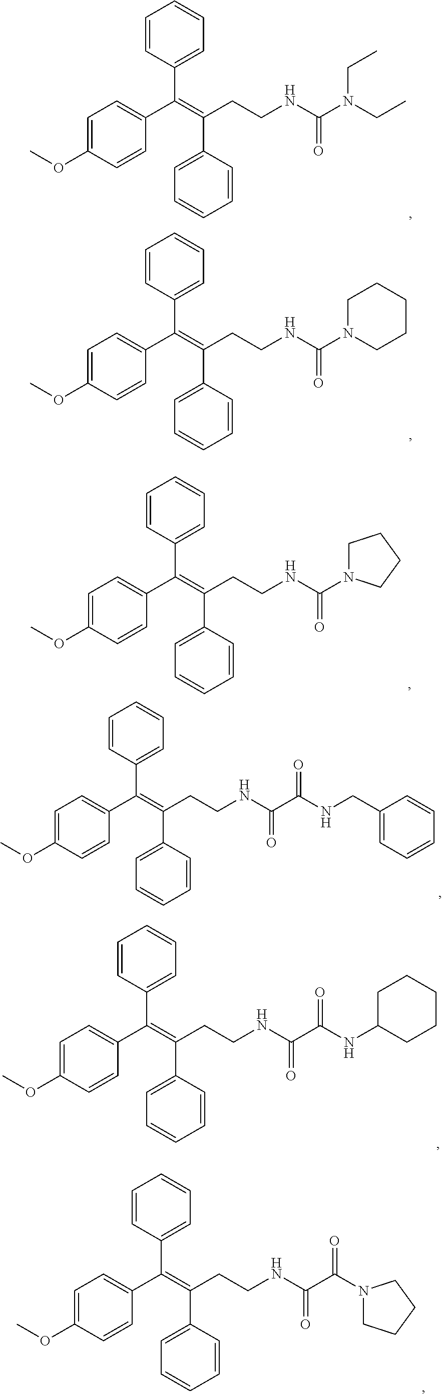 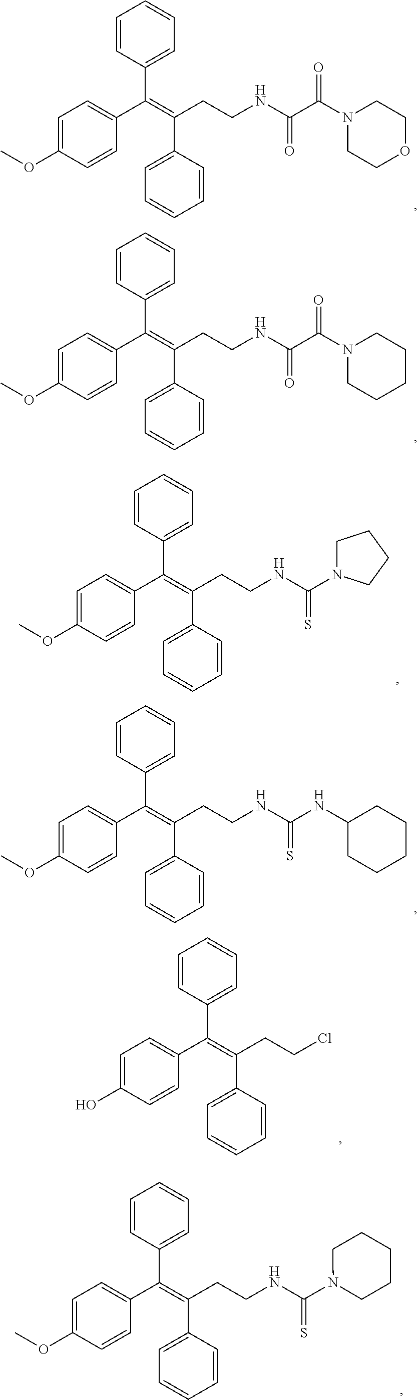 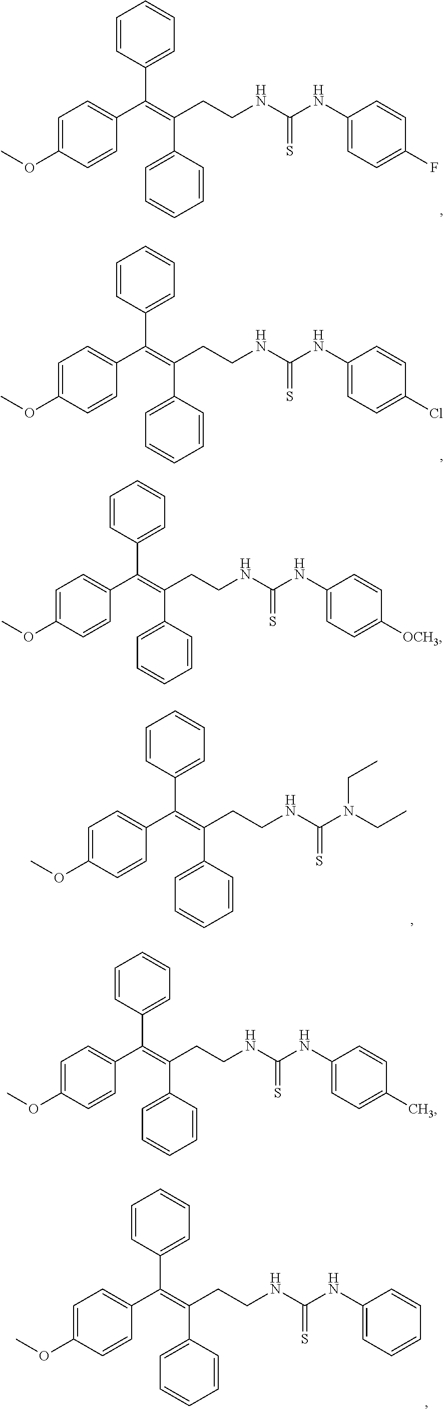 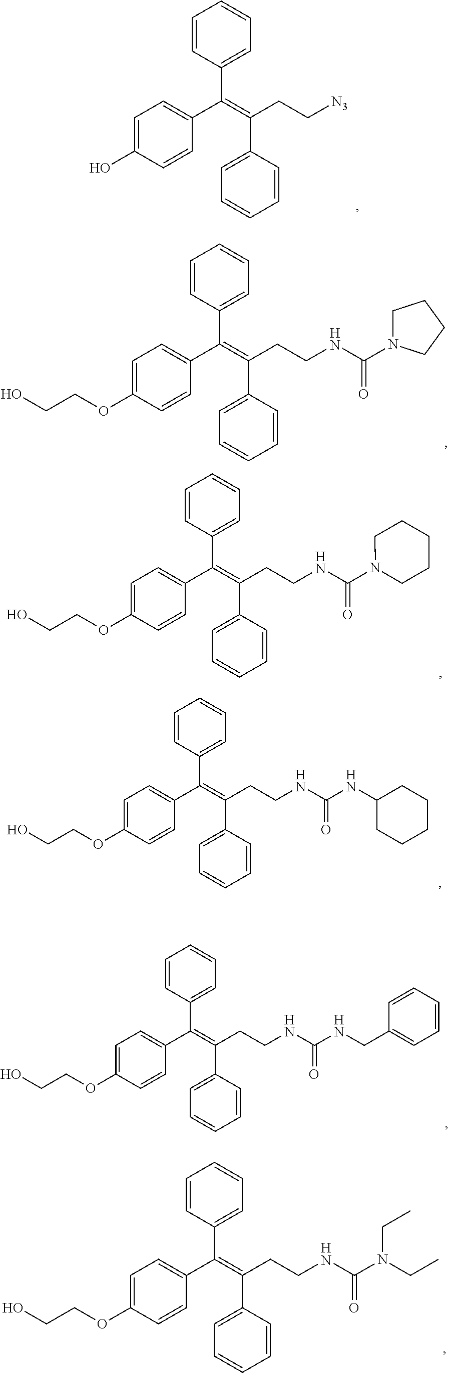 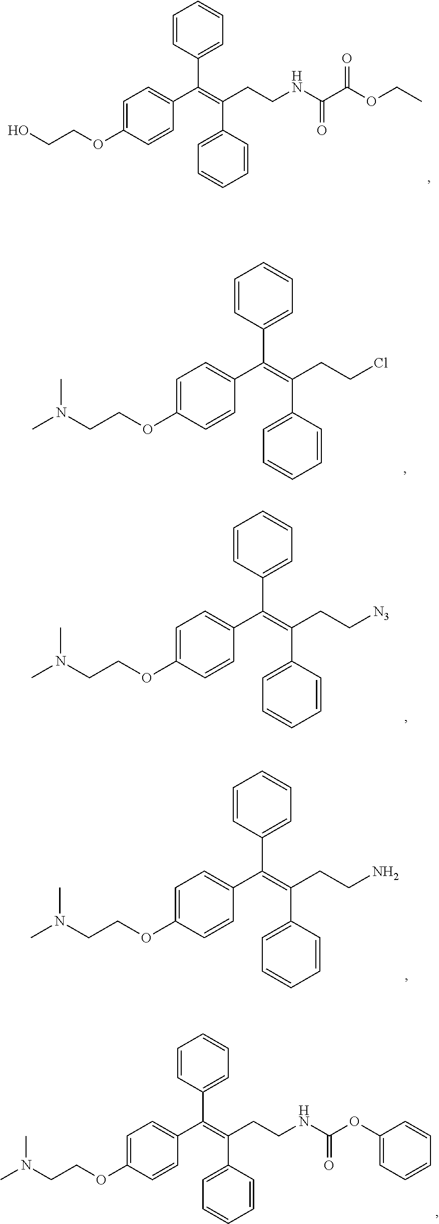 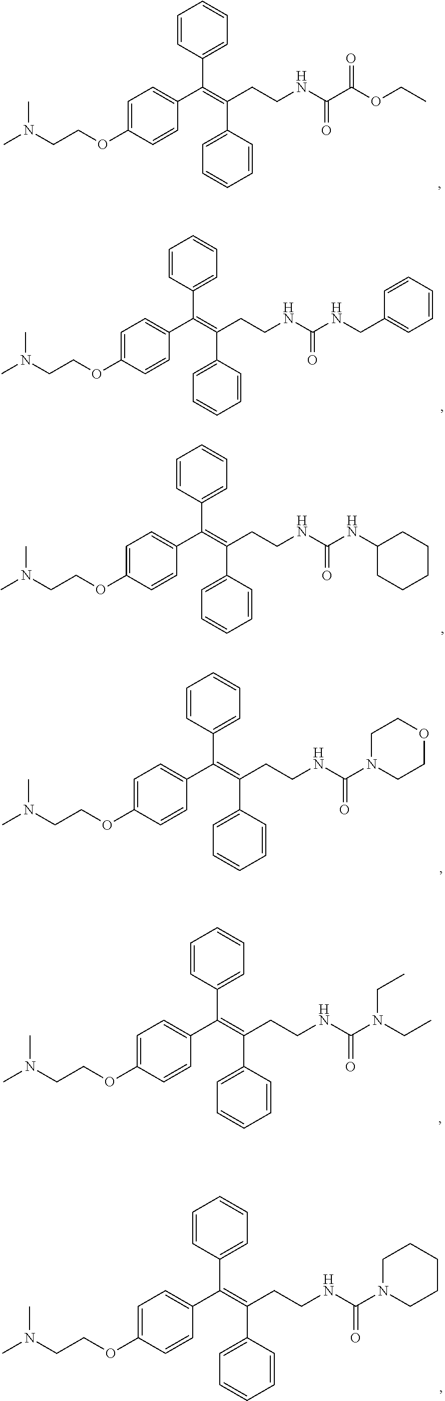 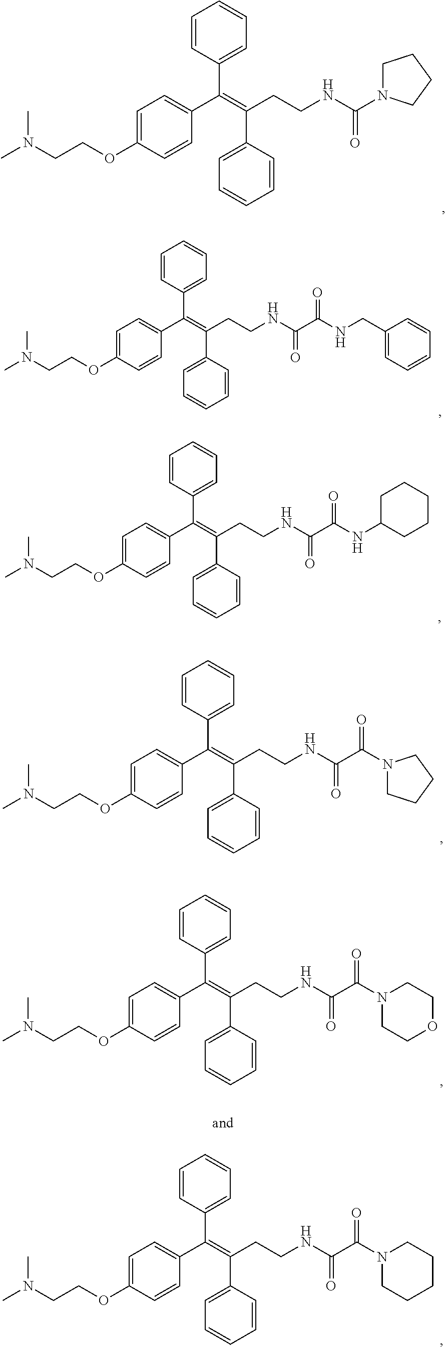
a salt or solvate thereof, and any combinations thereof.

7. A composition comprising a compound of claim 1.

8. The composition of claim 7, wherein the composition further comprises a pharmaceutically acceptable carrier.

9. The composition of claim 7, wherein the composition further comprises an additional therapeutic agent.

10. A method of preventing or treating cancer in a subject in need thereof, the method comprising administering to the subject a therapeutically effective amount of a composition comprising at least one compound of formula (I): 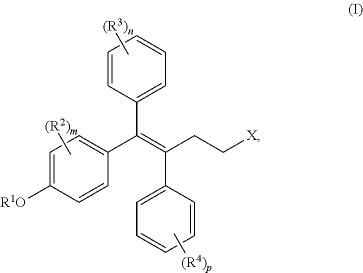
wherein in formula (I):
R¹ is selected from the group consisting of H and alkyl, wherein the alkyl group is optionally substituted;
each occurrence of R², R³, and R⁴ is independently selected from the group consisting of H, —C₁-C₆ alkyl, —C₁-C₆ fluoroalkyl, heteroalkyl, F, Cl, Br, I, —CN, —NO₂, —SR⁴, —S(═O)R⁴, —S(═O)₂R⁴, —NHS(═O)₂R⁴, —C(NH)(NH₂), —C(═O)R⁴, —OC(═O)R⁴, —CO₂R⁴, —OCO₂R⁴, —CH(R⁴)₂, —N(R⁴)₂, —C(═O)N(R⁴)₂, —OC(═O)N(R⁴)₂, —NHC(═O)NH(R⁴), —NHC(═O)R⁴, —NHC(═O)OR⁴, —C(OH)(R⁴)₂, and —C(NH₂)(R⁴)₂;
X is selected from the group consisting of N₃, N(R⁵)(R⁶), Cl, Br, I, and F;
R⁵ and R⁶ are each independently selected from the group consisting of H, —C₁-C₆ alkyl, —C(O)R⁷, and —C(S)R⁷;
R⁷ is selected from the group consisting of OR⁸, N(R⁸)(R⁹), C(O)R⁸, and C(O)N(R⁸)(R⁹);
R⁸ and R⁹ are each independently selected from the group consisting of hydrogen, —C₁-C₆ alkyl, aryl, cycloalkyl, and —C₁-C₆ alkyl-aryl, wherein the alkyl, aryl, cycloalkyl, or alkylaryl group may be optionally substituted, and wherein R⁸ and R⁹ may combine to form a ring, wherein the ring may optionally contain two or more heteroatoms;
m is an integer from 0 to 4;
n is an integer from 0 to 5; and
p is an integer from 0 to 5,
a salt or solvate thereof, and any combinations thereof.

11. The method of claim 10, wherein R¹ is selected from the group consisting of H, methyl, —(CH₂)₂OH, —(CH₂)₂OS(O)₂CH₃, —(CH₂)₂N₃, —(CH₂)₂NH₂, and —(CH₂)₂N(CH₃)₂.

12. The method of claim 10, wherein X is N(R⁵)(R⁶).

13. The method of claim 12, wherein either R⁵ and R⁶ are each H or R⁵ is H and R⁶ is —C(O)R⁷.

14. The method of claim 10, wherein m is 0, n is 0, and p is 0.

15. The method of claim 10, wherein the compound is selected from the group consisting of: 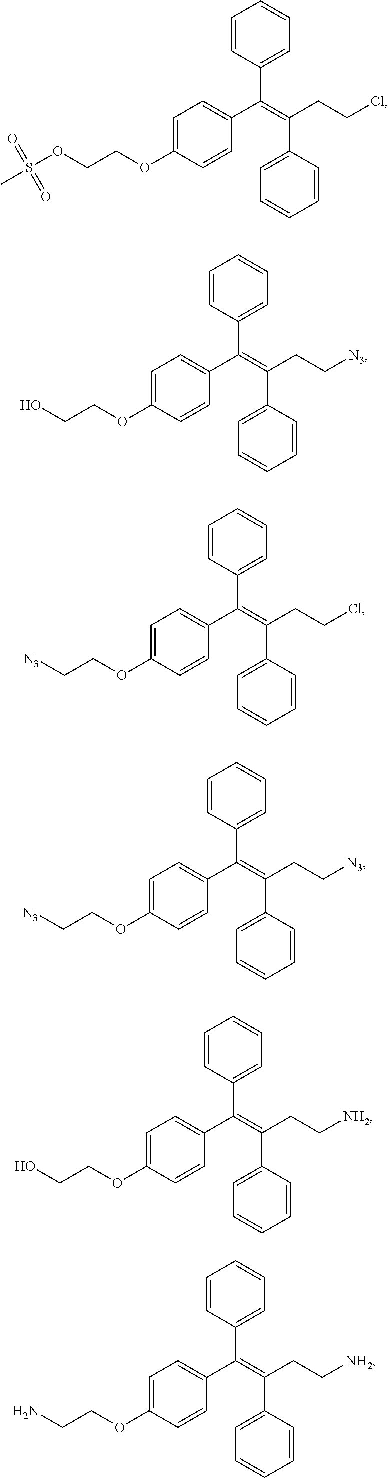 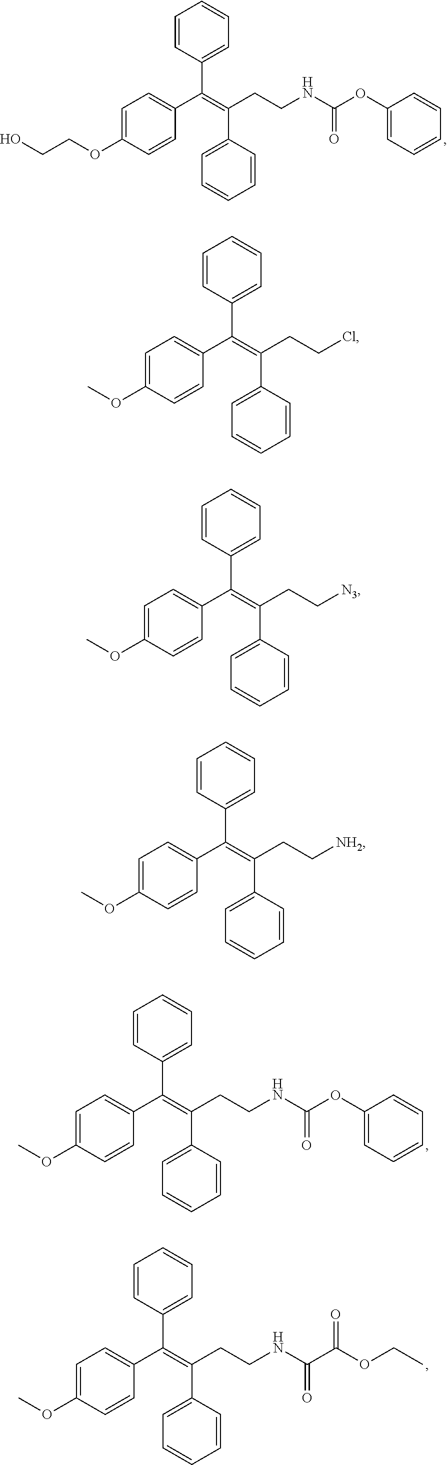 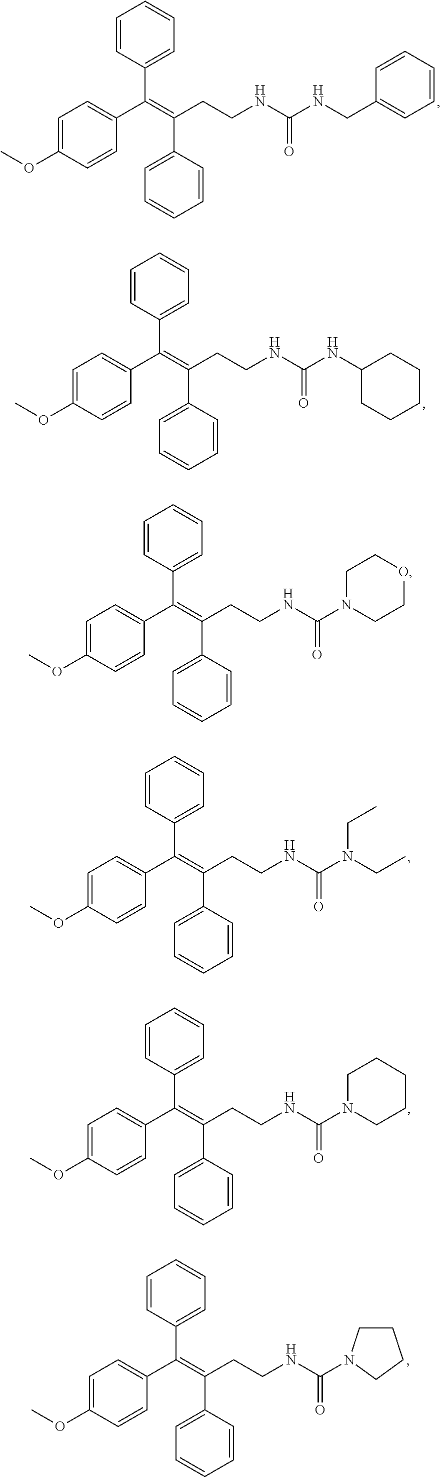 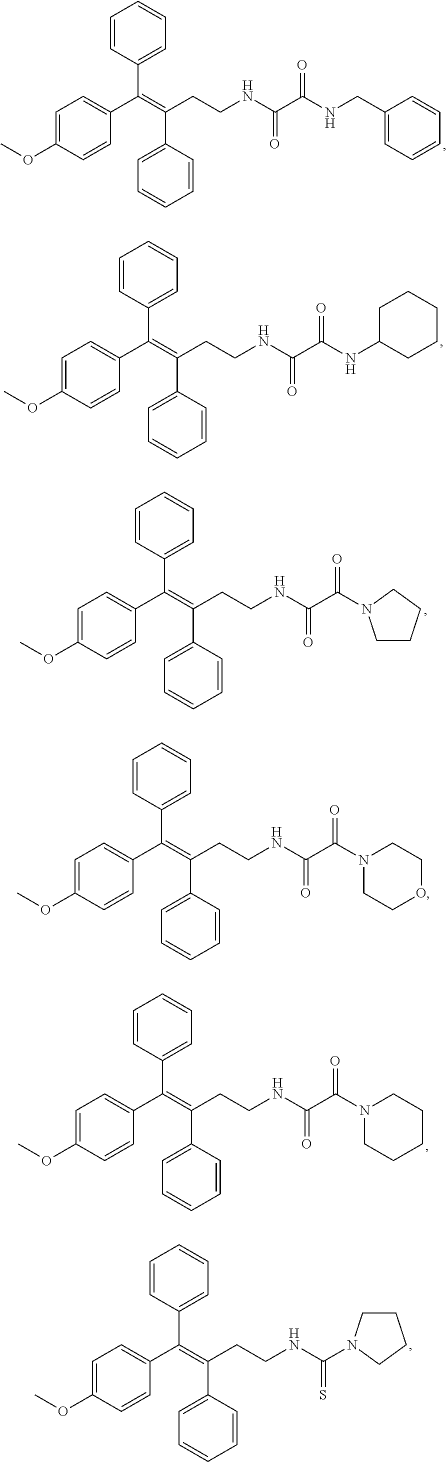 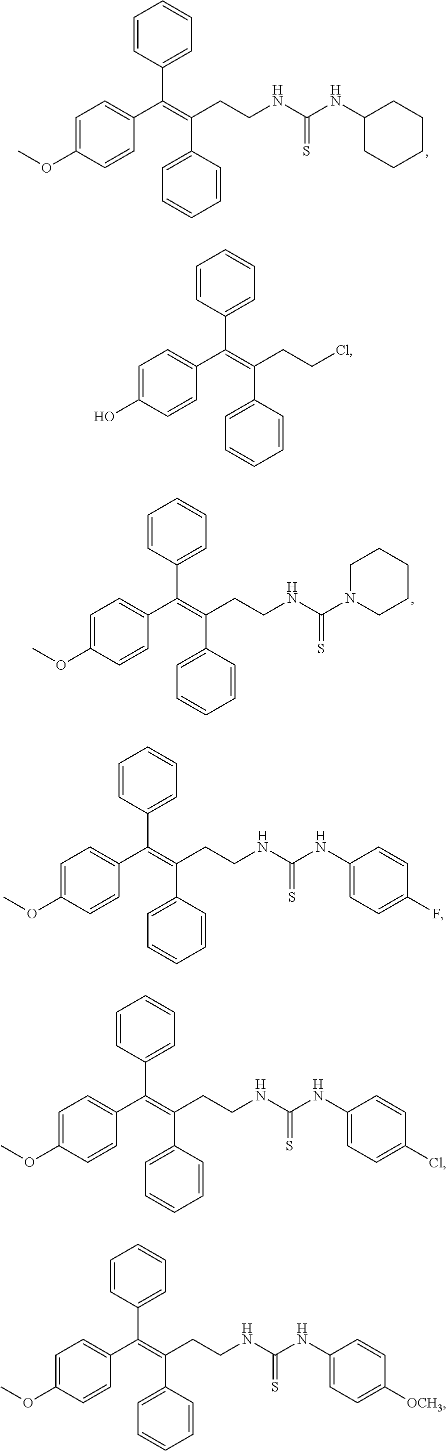 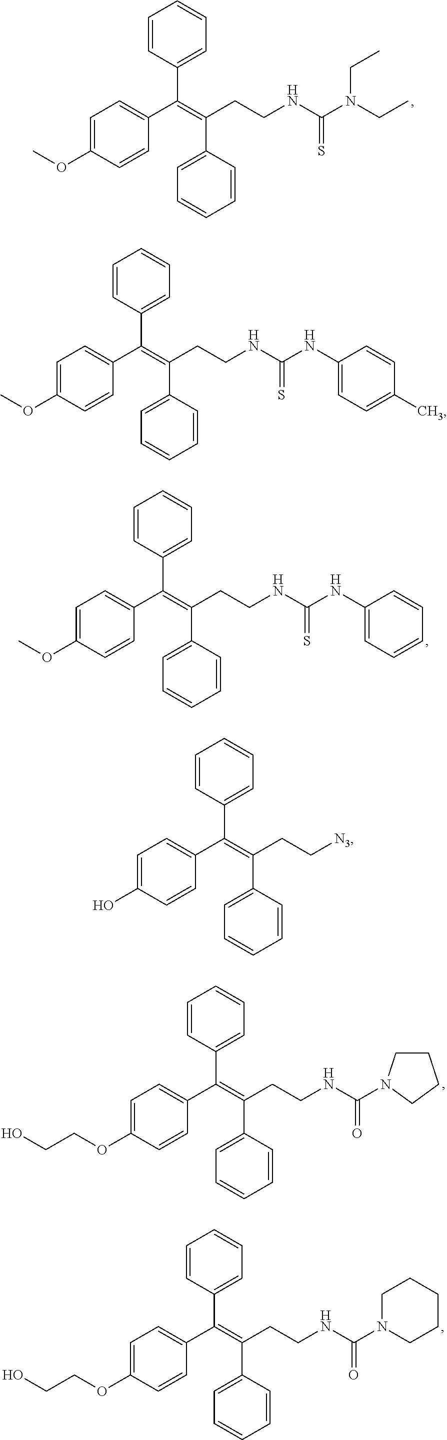 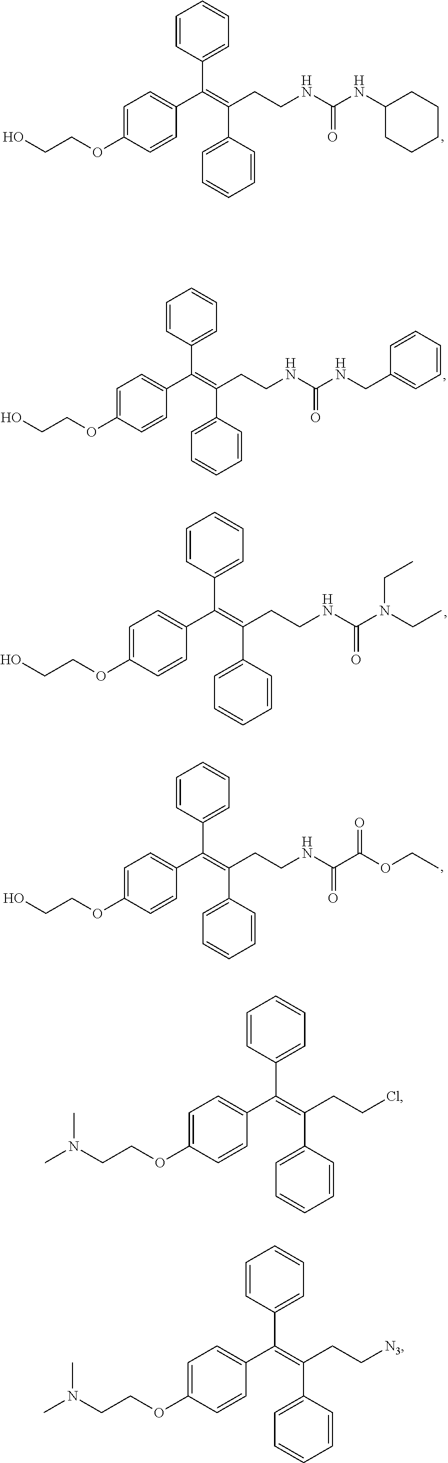 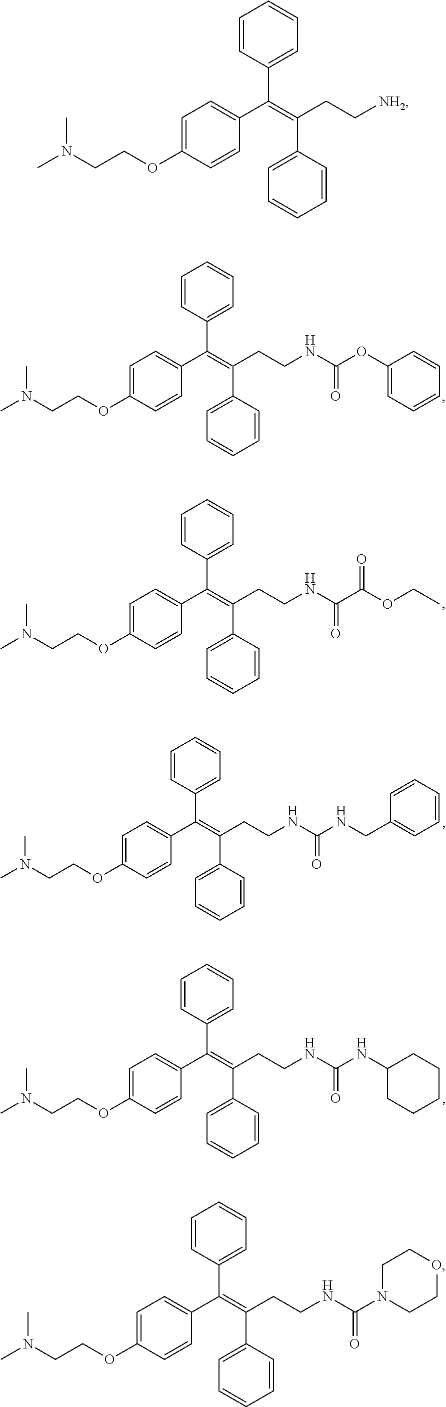 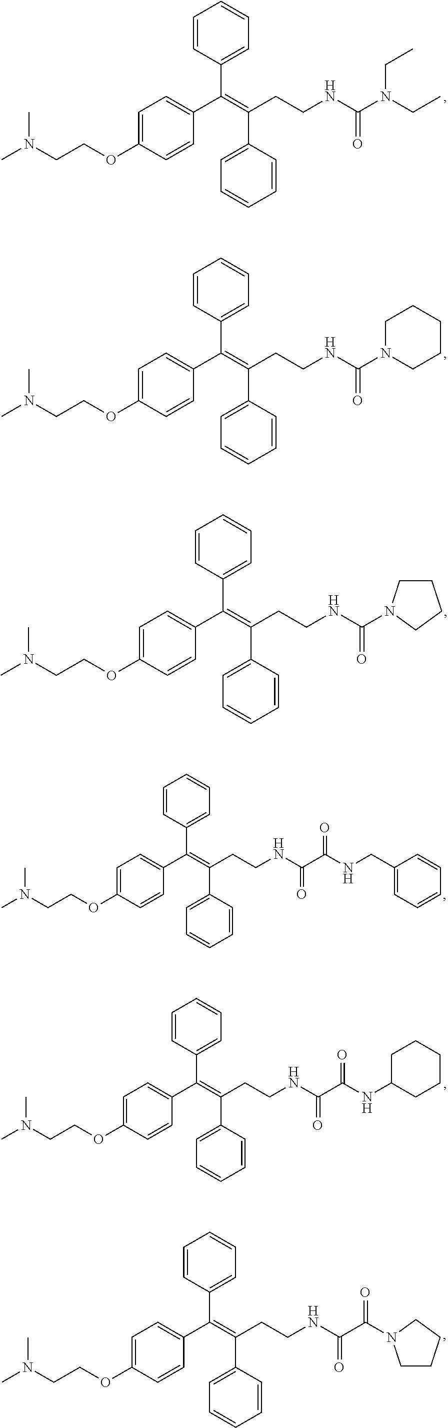 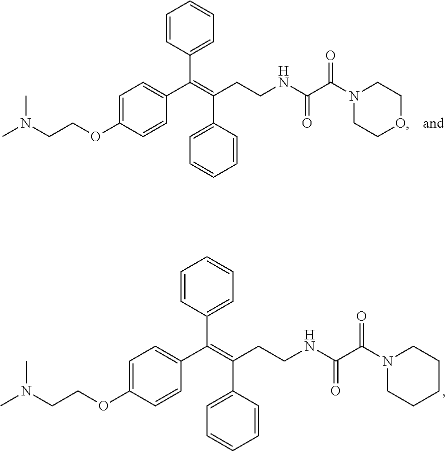 [SMILES: CN(C)CCOc1ccc(/C(=C(/CCNC(=O)C(=O)N2CCOCC2)c2ccccc2)c2ccccc2)cc1 ; CN(C)CCOc1ccc(/C(=C(/CCNC(=O)C(=O)N2CCCCC2)c2ccccc2)c2ccccc2)cc1]
a salt or solvate thereof, and any combinations thereof.

16. The method of claim 10, wherein the cancer is selected from the group consisting of lung cancer, colon cancer, melanoma, breast cancer, ovarian cancer, prostate cancer, liver cancer, pancreatic cancer, a CNS tumor, neuroblastoma, leukemia, bone cancer, intestinal cancer, lymphoma, and combinations thereof.

17. The method of claim 10, wherein the method further comprises administering to the subject at least one additional therapeutic agent.

18. The method of claim 17, wherein the therapeutic agent is a chemotherapeutic agent.

19. The method of claim 17, wherein the composition and the additional therapeutic agent are co-administered.

20. The method of claim 19, wherein the composition and the additional therapeutic agent are co-formulated.
